SEQUENCE LISTING (86 sequences)

SEQ ID NO: 1 - DNA, 7382 bp, Mus musculus
  CDS (133)..(6531)
ccccccgcgcagagcatccctgctgcagcgcagcaagacccggggctggcagccccaggaatccctagctgcttcgcagggataaaggactgaagttcttggaggagcgagtccaactcttcaagctgaactatgaccactttactcttggtctttgtgactctgagggtcatcgctgcagtgatctcagaagaagttccagaccatgacaactcactgagcgtgagcatccctcaaccatccccattgaaggtcctcctagggtcttccctcaccatcccctgctacttcatcgaccccatgcatcctgtgaccactgccccctccactgcccccctcaccccaagaatcaagtggagccgtgtttccaaggaaaaggaggtggtactgctggtggccactgaaggacaggttcgagtcaacagcatctaccaagacaaggtgtcgctccccaactatccagccatccccagcgatgctaccttggagatccagaaccttcgctccaatgactctgggatctaccgctgtgaagtgatgcacggcatcgaggacagcgaagccaccctggaggtcatagtgaaaggtattgtgttccactacagagctatttccacacgctacaccctggactttgatcgagcacagcgggcttgcctacagaacagcgccatcatcgccaccccagaacaactgcaggctgcctatgaggatggcttccaccagtgcgatgcaggctggctggctgaccagacagtcagataccccatccacacgccccgggaaggttgctatggtgacaaggacgagttccctggagtgagaacctacggaatccgggacaccaatgagacctatgatgtgtactgcttcgctgaggagatggagggtgaggtcttttatgcgacatccccagagaaattcaccttccaggaggcagccaacgagtgccggaggctgggggcacgcttggccaccacaggccagctctacctggcctggcagggcggtatggacatgtgcagcgctggctggctggcggaccgcagcgtgcgctaccccatctccaaggctcggcccaactgcgggggcaacctcctgggtgtaaggactgtctatctacacgccaaccagacaggctatcctgatccctcatcccgatatgacgccatctgttacacaggtgaagattttgtagacatcccagaaaacttcttcggggtgggtggtgaagacgacatcaccatccagacggtgacctggccagacctggagctgcccttgccccgtaatgtcacagagggagaggccctgggcagcgtgatcctcacggcaaagcccatctttgacctgtcccccactatctcagagccgggggaggccctcacacttgcccctgaagtggggagcacagccttcccagaggctgaggagagaactggagaagccaccagaccctggggctttcctgcagaagtcacacgtgggccggactctgccactgcctttgccagtgaggacctggtagtgcgagtgaccatctctccaggtgcagctgaagtccctggtcagccccacttgccagggggagttgtattccactatcgcccaggctccaccagatactctctgacatttgaggaggcacagcaggcctgcatgcacaccggggccatcattgcctctcctgagcagctccaagctgcctatgaggcaggctatgagcagtgtgatgctggctggctgcaggaccagaccgtcagataccccattgtgagcccacgaaccccatgtgtgggtgacaaagacagcagcccaggagtcaggacctaccgcgtgcgcccatcatcagaaacctatgatgtctactgctacgtggacaagcttgagggggaagtgttcttcgccacacgcctcgagcagttcaccttccaggaagcgcgggccttctgtgcggctcaaaatgccaccctggcctccaccggccagctctatgctgcctggagccagggtctggacaagtgctatgctggctggctggcagatggcaccctccgataccccatcatcacccctcggcctgcctgtggtggggacaaacctggcgtgagaactgtctacctctaccccaaccaaaccggcctccctgacccactgtcaaagcaccatgccttctgcttccgaggtgtgtcagtggcgccctctccaggagaagaagagggtagtacacccacatcaccctctgacatagaggactggatcgtcactcaggtggggcctggtgtggatgctgtccccttggagccaaagacaacagaagtgccatatttcaccactgagccaagaaaacagactgaatgggagccagcctacaccccagtgggcacatccccacagccagggatccctcccacatggcttcccaccctcccagcagcagaggaacacacagaaagcccctctgcctctgaagagccctctgcctcagcagtcccttccacctcagaggagccatacacatcttcatttgcagtgccgagcatgacagagctgccaggctctggggaggcgtcgggcgcacctgacctcagtggtgacttcacaggcagtggagatgcttcaggacgccttgactccagtgggcagccttcagggggcattgaaagtggccttccctcaggtgaccttgactccagtggcctcagccccacagtgagctcaggcctgcctgtagaaagtggttctgcctcaggagatggagaagtcccctggtcccatactcccacagttggcaggttgccctctggaggtgagagccccgaaggctctgcctctgcctctggaacaggagaccttagtgggctgccttcaggaggagaaattacagaaacttctacttctggggcagaagaaaccagtggacttccttctggaggagacggtctagaaacttctacctctggagtagatgatgtcagtggaattcctactggaagagaaggtctagagacttctgcctctggagtagaggacctcagtggacttccttctggagaagaaggttcagaaacatctacctctggaatagaggacatcagtgtacttccaactggaggagaaagtctagaaacctctgcttctggagtgggagacttgagtggacttccctcaggaggagaaagtctagaaacatctgcttcaggtgcagaggatgtcactcagcttcctactgaaagaggaggtctagagacttctgcctctggagtagaagacatcactgttcttcctactggaagagaaagtctagaaacttctgcctctggagtagaggatgtcagtggacttccttctggaagagaaggtctagagacttctgcctctggaatagaggacattagtgtgtttcctactgaagcagaaggtctggacacttctgcctctgggggatatgttagtgggattccttctggaggagatggtacagaaacctctgcttctggagtagaggatgtgagtggtcttccatctggaggagagggtctagaaacttctgcctctggagtggaagatcttggtccttctactagagatagtctagagacatctgcttcaggagtagatgttactgggtttccttctggaagaggggacccagagacctctgtttctggggtaggtgatgacttcagtggacttccttctggaaaagaaggcctggagacctcagcttctggagctgaggacctcagtggcttgccctctggaaaagaagacttggtagggtctgcttctggggccttggactttggcaaactacctcctggaactctaggaagtggtcaaactccagaagtaaatggctttccctctggatttagtggtgagtattctggagcagacattggaagtggcccatcctctggcctgcctgactttagtggacttccatctggctttccaactgtctcccttgtggacagtaccttagtggaagtgatcacagccaccacttccagtgaactggaaggaagggggaccattggcatcagtggttcaggagaagtatcagggctgcccctgggtgaattggacagtagtgcggacattagtggtctcccttcaggaactgaactcagtggccaagcatctggatctcctgatagcagtggagaaacatctggattttttgatgttagtggacagccatttgggtcttctggcgtcagcgaggaaacatctgggattcctgaaatcagtgggcagccatcagggactcctgacaccactgcgacatctggagtgactgagcttaatgaactgtcctctggacaaccagatgtcagtggagatgggtctggaattctctttggcagtggacagtcctctggtataacatctgtgagtggagaaacctctgggatttctgatctcagtgggcagccctcagggttcccagtgttcagtggaacagcaaccagaacccctgacctggcttctggcaccataagtggcagtggagagtcttctggcattacatttgtggacaccagttttgttgaagtgacccctaccacatttagggaagaagaagggttaggatctgtggaactcagtggctttccttctggggagacggaactgtctggcacatctgggacggtggacgtcagtgaacaatcttctggagcaattgattccagtggactcacatcccccactccagagttcagtggcctcccaagtggagtagctgaggtcagtggtgaattctctggagttgagactgggagcagcttgccctcaggagcatttgatggcagtggacttgtctcaggtttccccactgtgtctcttgtagacagaactttggtggaatctataactcaggctcctactgctcaagaagctggagaaggaccttcgggcattttggaattcagtggtgcccattctgggacaccagacatatctggggagctttctgggtctctggacctaagcacattgcagtctgggcagatggaaaccagcacggagacaccaagctctccatattttagtggagacttttccagcaccactgatgtaagtggagaatccatagctgccacaactggcagtggggaaagctctgggcttccggaagttactttaaacacctcagagttagtggagggtgtgactgaacccactgtttcccaggaacttggccatggtccttctatgacatacatctcccggctctctgaggccagtggggacgcctcagcatccggggaccttggtggcgctgtaacaaacttcccagggtctggagtagaagcttcagtcccagaagccagcagtgacctgtctgcttaccctgaggctggtgtgggagtgtctgctgcccctgaggccagcagtaaactgtctgagttcccagatctgcatggaatcacttctgccttccatgaaacagatctggaaatgacaaccccaagcacagaggtaaacagcaacccatggacctttcaggaaggcaccagggagggatcggccgctccggaagtgagtggagaatctagcactacctccgacatagacacaggcacttcaggggtgccttctgccacacccatggcttctggagacaggactgaaatcagcggagaatggtctgatcacacctcagaggtgaatgttgccatcagcagcaccatcacagagtccgagtgggcccagcctacccggtaccctacagagacacttcaagaaatcgaatccccaaatccctcatactcaggagaggagacccagacagcagaaacaaccatgtccctgacagatgcccccaccctctcttcttcagaagggtcaggggagacggagtcaaccgttgcagaccaggagcaatgtgaggaggggtggaccaagttccagggtcactgttaccgccactttcctgaccgagagacctgggtggatgcggagagacggtgtcgggagcagcagtcacatctgagcagcattgtcactcctgaggaacaggagttcgtcaacaaaaatgctcaagactaccagtggatcggtctgaatgacaggactatcgaaggggacttccgctggtctgacggacactctctgcaatttgagaagtggcgtccaaaccagcctgacaacttctttgccaccggagaggactgtgtggtgatgatctggcatgagagaggcgaatggaacgacgtcccctgcaattaccagctgcccttcacgtgtaaaaagggcaccgtggcctgtggagaccccccagtggtggagcatgctagaaccctcgggcagaagaaagatcgctacgagatcagctccctggtgcggtaccagtgcactgagggctttgtccagcgccacgtgcccaccatccggtgccagcccagcgggcactgggaagagcctcgaatcacctgcacagaccccaacacctacaagcacaggctacagaagcggacgatgagacccacacggaggagccgccccagcatggcccactgagaggagcttccataatgtgcccaggatgctgagcccagcggccagccaggctgaccgtgcatcccacccacatggtgtcttcttgtcgctttttgtcatataaggaatccattaaagaaggaaaaaaaacccacattgtgtgtgccccactccccgcatcacccaaactgcatctaatttatccccttctcccccccccccaaaaaaaaaaagaaaagaaatgcccaagcaagtcactgtaaccccgtggtacctcagctttgagacttttttgatttccccacccgtgccctttcccagggaccagtgcaggcacagggtgagaaggtaaggggttaaattaaataaagaagattcttttttgttgttgtttcctgactttgcccaagaacagtacaatggttggttacttcgcctccagggagagctaggggcccagaggaggagctgaaagaagacaggaccaggaagggaggagtagggcggtcacagagcttggaggactcagaagaggcaaagtacagcttagtgtgttggacaaaaggaattgaggtctgtgccatctgtgagggaaggacctggggcagggacaggctggctgaagagaagcagagccctctcctagggacctttcatctgggtcaaccaacctagcaggtgtcatttctcggctggactgggtaggggcacttccttcccaggggtctccgccgtccccttccctctccccaccccgggacggtgcaggcaccagtgttccgtgcacctatttatatttttgaaaactaaagattatgataattataataataaagacattggaagagatctaaaaaaaaaaaaaaaaaaaa

SEQ ID NO: 2 - protein, 2132 aa, Mus musculus
MTTLLLVFVTLRVIAAVISEEVPDHDNSLSVSIPQPSPLKVLLGSSLTIPCYFIDPMHPVTTAPSTAPLTPRIKWSRVSKEKEVVLLVATEGQVRVNSIYQDKVSLPNYPAIPSDATLEIQNLRSNDSGIYRCEVMHGIEDSEATLEVIVKGIVFHYRAISTRYTLDFDRAQRACLQNSAIIATPEQLQAAYEDGFHQCDAGWLADQTVRYPIHTPREGCYGDKDEFPGVRTYGIRDTNETYDVYCFAEEMEGEVFYATSPEKFTFQEAANECRRLGARLATTGQLYLAWQGGMDMCSAGWLADRSVRYPISKARPNCGGNLLGVRTVYLHANQTGYPDPSSRYDAICYTGEDFVDIPENFFGVGGEDDITIQTVTWPDLELPLPRNVTEGEALGSVILTAKPIFDLSPTISEPGEALTLAPEVGSTAFPEAEERTGEATRPWGFPAEVTRGPDSATAFASEDLVVRVTISPGAAEVPGQPHLPGGVVFHYRPGSTRYSLTFEEAQQACMHTGAIIASPEQLQAAYEAGYEQCDAGWLQDQTVRYPIVSPRTPCVGDKDSSPGVRTYRVRPSSETYDVYCYVDKLEGEVFFATRLEQFTFQEARAFCAAQNATLASTGQLYAAWSQGLDKCYAGWLADGTLRYPIITPRPACGGDKPGVRTVYLYPNQTGLPDPLSKHHAFCFRGVSVAPSPGEEEGSTPTSPSDIEDWIVTQVGPGVDAVPLEPKTTEVPYFTTEPRKQTEWEPAYTPVGTSPQPGIPPTWLPTLPAAEEHTESPSASEEPSASAVPSTSEEPYTSSFAVPSMTELPGSGEASGAPDLSGDFTGSGDASGRLDSSGQPSGGIESGLPSGDLDSSGLSPTVSSGLPVESGSASGDGEVPWSHTPTVGRLPSGGESPEGSASASGTGDLSGLPSGGEITETSTSGAEETSGLPSGGDGLETSTSGVDDVSGIPTGREGLETSASGVEDLSGLPSGEEGSETSTSGIEDISVLPTGGESLETSASGVGDLSGLPSGGESLETSASGAEDVTQLPTERGGLETSASGVEDITVLPTGRESLETSASGVEDVSGLPSGREGLETSASGIEDISVFPTEAEGLDTSASGGYVSGIPSGGDGTETSASGVEDVSGLPSGGEGLETSASGVEDLGPSTRDSLETSASGVDVTGFPSGRGDPETSVSGVGDDFSGLPSGKEGLETSASGAEDLSGLPSGKEDLVGSASGALDFGKLPPGTLGSGQTPEVNGFPSGFSGEYSGADIGSGPSSGLPDFSGLPSGFPTVSLVDSTLVEVITATTSSELEGRGTIGISGSGEVSGLPLGELDSSADISGLPSGTELSGQASGSPDSSGETSGFFDVSGQPFGSSGVSEETSGIPEISGQPSGTPDTTATSGVTELNELSSGQPDVSGDGSGILFGSGQSSGITSVSGETSGISDLSGQPSGFPVFSGTATRTPDLASGTISGSGESSGITFVDTSFVEVTPTTFREEEGLGSVELSGFPSGETELSGTSGTVDVSEQSSGAIDSSGLTSPTPEFSGLPSGVAEVSGEFSGVETGSSLPSGAFDGSGLVSGFPTVSLVDRTLVESITQAPTAQEAGEGPSGILEFSGAHSGTPDISGELSGSLDLSTLQSGQMETSTETPSSPYFSGDFSSTTDVSGESIAATTGSGESSGLPEVTLNTSELVEGVTEPTVSQELGHGPSMTYISRLSEASGDASASGDLGGAVTNFPGSGVEASVPEASSDLSAYPEAGVGVSAAPEASSKLSEFPDLHGITSAFHETDLEMTTPSTEVNSNPWTFQEGTREGSAAPEVSGESSTTSDIDTGTSGVPSATPMASGDRTEISGEWSDHTSEVNVAISSTITESEWAQPTRYPTETLQEIESPNPSYSGEETQTAETTMSLTDAPTLSSSEGSGETESTVADQEQCEEGWTKFQGHCYRHFPDRETWVDAERRCREQQSHLSSIVTPEEQEFVNKNAQDYQWIGLNDRTIEGDFRWSDGHSLQFEKWRPNQPDNFFATGEDCVVMIWHERGEWNDVPCNYQLPFTCKKGTVACGDPPVVEHARTLGQKKDRYEISSLVRYQCTEGFVQRHVPTIRCQPSGHWEEPRITCTDPNTYKHRLQKRTMRPTRRSRPSMAH

SEQ ID NO: 3 - DNA, 2291 bp, Mus musculus
  CDS (257)..(1363)
aagaggtaatccagaactcaaggagcgcgggcgcagcagctgtgagcattaggtgctgtggccaagcgctgggacaaggatctgccgctaccagcgcatcctctctcctctccagctctgtcccgcactcgctgcacccttccccaggccaccgcgcttcctatgtgatcttccggggcaacgcggagccctttctcacagctcagcagtgaatttccaccccgaaaactgcagtaagccgcctttcaaggacaagatgttgataaatatgaaaggcatcttatggatgtgctcaaccttattactaacccatgcactacatcaagccaaaatggaaaccagcccacctgttaaaggctctctgtctggaaaagtggtcctaccttgtcatctttcaaccttacctaccttaccacccaattacaacacgagtgaatttctcagaatcaaatggtctaagatggaagtggacaaaaatggaaaagatataaaggagacgactgtcttggtggcccagaacggaaatatcaagattggtcaggactacaaggggcgagtgtccgtgcctacacatcccgatgatgtaggtgatgcttccctcaccatggtcaaactccgggctagtgatgcaggcgtctaccgatgtgatgtcatgtatgggattgaagacactcaggacaccatgtcactggctgtggatggtgttgtgtttcactacagggcagccaccagcaggtacactctgaactttgccgctgctcaacaggcttgtttggatatcggggcggtcatagcaagcccagagcagctgtttgccgcctatgaggatggatttgagcagtgtgatgcaggatggctgtctgatcaaactgtcagatatcccatacgggctccccgagagggctgttacggagacatgatggggaaggaaggggttcggacctatggattccgctctccccaggaaacctatgatgtgtattgttatgtggatcatctggatggcgatgtgttccacatcactgctcccagtaagttcaccttcgaggaggccgaagcagagtgtacaagcagggatgcgaggctggcgactgttggagaacttcaggcagcttggagaaatggctttggccaatgcgattacggctggctgtcggatgccagcgtgcggcaccctgtgactgtggccagggcccagtgtggaggaggtctacttggggtgagaaccctgtatcgttttgagaaccagacatgcttccctctccctgatagcagatttgatgcctactgctttaaacgtaagtgtttgatatctcttttaaaaatacagattaaaaagctgcagcaaatatatacccactgaggttccagtggatgtcgggatggattctgtattctgtactaagtgcatggaggacctacagtgcagtgacacaaacagacttaataccaccattatggttaacacagtagggagtttggcaagtgggattttttttgggaggtggggagataacaacgtcagagtggggtcttaactgcaccccagaattccaaaaatcacagaacacaccctgaattgaaatttcttattaattgaagcctttcaatgcaatttcactcttaatataacaatgttaagacaacagcagctgagttaaacctttgaatcctgttttattttttgatatgattcttattataaaaaggagacaatttaagcttaagtcattgcaggtgagtatttgtgctgaaattaataatgattgtgttcaattcctacctaaaaatgcatcatactatcaaataatacgattcacatatctcattttgtttcaagtaatagaataggcgccttttggcgattaatcttgctttgaattaattaaagtacaatacagcctccttgcatgaattggctatgctcagttagatttgccttgtaaaattaattttaggtagcattcattacatatatatatatgtatatatatatattagacattcttaaattattttaaaatattagtcacatacagttttatagttgtagagtgcttaactaaactaataactagtcaatattttattgtaatatgtactttaggatcggtaagatgttagctaacaaatgcctccatactttagcaacagaggccaagtctgacatcgagtatcagaacaatgaaaagccaatgtaacacatgaagtcttttaaaccagctttctaattccttttgttagatcaatgggtaattaaaatagcttgaaaatgtaaaaaaaaaaaaaaa

SEQ ID NO: 4 - protein, 368 aa, Mus musculus
MLINMKGILWMCSTLLLTHALHQAKMETSPPVKGSLSGKVVLPCHLSTLPTLPPNYNTSEFLRIKWSKMEVDKNGKDIKETTVLVAQNGNIKIGQDYKGRVSVPTHPDDVGDASLTMVKLRASDAGVYRCDVMYGIEDTQDTMSLAVDGVVFHYRAATSRYTLNFAAAQQACLDIGAVIASPEQLFAAYEDGFEQCDAGWLSDQTVRYPIRAPREGCYGDMMGKEGVRTYGFRSPQETYDVYCYVDHLDGDVFHITAPSKFTFEEAEAECTSRDARLATVGELQAAWRNGFGQCDYGWLSDASVRHPVTVARAQCGGGLLGVRTLYRFENQTCFPLPDSRFDAYCFKRKCLISLLKIQIKKLQQIYTH

SEQ ID NO: 5 - DNA, 2583 bp, Mus musculus
  CDS (162)..(2339)
cggcagtggctggcaagaaacaattctgcaaaaataatcatacccagcctggcaattgtctgctcctcggtccattgctccgccgccgtccacagtcgcttgcaaggggaaggcactgaatttaccgcggccagaacatccctcccagccggcagtttacaatgctgcgaactaaggatctcatctggactttgtttttcctgggaactgcagtttccctgcaggtagatattgttcccagccaaggagaaatcagcgttggagagtccaaattcttcctgtgtcaagtggcaggagatgccaaagataaggacatctcctggttctcccccaatggggagaagctgagcccaaaccagcagcggatctcagtggtgtggaatgatgacgactcctctaccctcaccatctacaacgccaacatcgacgatgccggcatatacaagtgtgtggtcaccgctgaggacggcacccagtctgaggccactgtcaacgtgaagatcttccagaagctcatgttcaagaatgcaccaaccccacaggagtttaaggaaggggaggatgctgtgattgtctgtgatgtggtcagctccctgcctccaaccatcatctggaaacacaaaggccgagatgtcattctgaaaaaagacgtccggttcatagtcctgtccaacaactacctgcagatcaggggcatcaagaaaacagatgagggtacttaccgctgtgagggcaggatcctggcccgcggggaaatcaacttcaaggacattcaggtcattgtgaatgtaccacccactgtccaggccagacagagcatcgtgaatgccactgccaacctgggccagtctgtcaccctggtgtgtgatgctgatggatttccagagcccaccatgagctggacaaaggatggggaacccatagagaacgaggaggaagatgagagaagcagatcttcagtgagtgacagctccgaggtgaccatcaggaatgtggataaaaacgacgaggccgaatacgtctgcatcgcagagaacaaggctggcgagcaggatgcctccatccacctcaaggtctttgcaaagcccaaaatcacctatgtagagaatcagacggccatggaactagaggagcaagtcactctgacctgtgaagcctccggagaccccatcccgtccatcacgtggagaacatccacccggaacatcagcagtgaagaacaggatctggatgggcacatggtggttcgcagccatgctcgtgtgtcctccttgaccctgaagagcatccagtacagagatgctggagaatacatgtgcactgccagcaacaccatcggccaggactcccagtccatcgacctcgaatttcaatatgctcccaagctccagggccctgtggctgtctacacctgggaagggaaccaagtgaacatcacctgtgaggtctttgcctatcccagtgccacaatctcctggttccgagatggtcagttgctgccaagctccaactacagcaatatcaagatctacaacaccccatctgctagctatctggaggtgaccccagattcagaaaatgactttggaaactacaactgtacagcagtgaaccgtattggacaggagtccttggaattcatccttgttcaagcagacacaccgtcttctccatccattgaccgggtggaaccatactccagcacagcgcaggtgcagtttgatgagccagaggccacagggggcgtgcccattctcaagtacaaggctgagtggaagtcgctgggcgaagaatcctggcatttcacgtggtatgatgccaaagaagccaacatggagggcattgtcaccatcatgggcctgaaacctgagacgacgtactcggatcgactggcggccctcaacggcaaggggctgggcgagatcatgcagccatctgagtccaagacacagccagttccggaactcagtgcacccaagctggaagggcagatgggagaggacgggaactccatcaaggtgaacctgatcaagcaggatgacggaggctcccccatcagacactatctggtcaagtacagagcgctcgcctctgagtggaaaccggaaatcaggctcccatccggcagtcaccacgtcatgctcaagtccctggactggaacgcagagtatgaagtctatgtggtagctgaaaaccagcaaggaaaatccaaggcagctcactttgtgttcaggacctcagcccagcccacggccatcccagccaccctgggcggaagctccacctcctacaccttggtctcattgcttttctctgcggtgactcttcttctgctctgatagaaacttgaacatgcatgtgtgcgagctctcatgtgcgcatgcgcacacgcacacacacgcacacgcacacgcacacgcacgcacacgcacacacacacacacacagacacacacacgcacacacacacgcacatgcacacgcgcacacacgcacacgcacacgcacacgcacgcacacacacacacacacacaccattgtatttgattaaaaagcccagttcctataaaaaaaaaaaaaaa

SEQ ID NO: 6 - protein, 725 aa, Mus musculus
MLRTKDLIWTLFFLGTAVSLQVDIVPSQGEISVGESKFFLCQVAGDAKDKDISWFSPNGEKLSPNQQRISVVWNDDDSSTLTIYNANIDDAGIYKCVVTAEDGTQSEATVNVKIFQKLMFKNAPTPQEFKEGEDAVIVCDVVSSLPPTIIWKHKGRDVILKKDVRFIVLSNNYLQIRGIKKTDEGTYRCEGRILARGEINFKDIQVIVNVPPTVQARQSIVNATANLGQSVTLVCDADGFPEPTMSWTKDGEPIENEEEDERSRSSVSDSSEVTIRNVDKNDEAEYVCIAENKAGEQDASIHLKVFAKPKITYVENQTAMELEEQVTLTCEASGDPIPSITWRTSTRNISSEEQDLDGHMVVRSHARVSSLTLKSIQYRDAGEYMCTASNTIGQDSQSIDLEFQYAPKLQGPVAVYTWEGNQVNITCEVFAYPSATISWFRDGQLLPSSNYSNIKIYNTPSASYLEVTPDSENDFGNYNCTAVNRIGQESLEFILVQADTPSSPSIDRVEPYSSTAQVQFDEPEATGGVPILKYKAEWKSLGEESWHFTWYDAKEANMEGIVTIMGLKPETTYSDRLAALNGKGLGEIMQPSESKTQPVPELSAPKLEGQMGEDGNSIKVNLIKQDDGGSPIRHYLVKYRALASEWKPEIRLPSGSHHVMLKSLDWNAEYEVYVVAENQQGKSKAAHFVFRTSAQPTAIPATLGGSSTSYTLVSLLFSAVTLLLL

SEQ ID NO: 7 - DNA, 3153 bp, Mus musculus
  CDS (151)..(2802)
  misc_feature (3043)..(3043): n is a, c, g, or t
gaggctcccggcgagctggcgcccctgtctgggtcccgcgcgcccggccctgctcgcgcccgcgcatcgcgccgcagtctcggtctgcggctgcgggacgtgacggcgtgcgcggaggggacctcgcaagttcttccatcagtgtgcagaatgataccactgcttctgtccctgctggccgctctggtcctgacccaagcccctgccgccctcgctgatgacctgaaagaagacagctcggaggatcgagccttccgcgtgcgcatcggtgccgcgcagctgcggggcgtgctgggcggtgccctggccatcccatgccacgtccaccacctgcggccgccgcgcagccgccgggccgcgccgggttttccccgggtcaagtggaccttcctgtccggggaccgggaggtagaggttctggtggctcgcgggctgcgcgtcaaggtaaacgaagcctaccggttccgcgtggcgctgcctgcctaccccgcatcgctcacggatgtgtctctagtattgagcgaactgcggcccaatgattccggggtctatcgctgcgaggtccagcacggtatcgacgacagcagtgatgctgtggaggtcaaggtcaaaggggtcgtcttcctctacagagagggctctgcgcgctatgctttctccttcgctggagcccaggaagcctgcgctcgcataggagcccgaatcgccaccccggagcagctctatgctgcctacctcggcggctatgagcagtgtgatgcaggctggctgtccgaccaaactgtgaggtaccccatccagaacccacgagaggcctgctctggagacatggatggctatcctggcgtgcggaactacggagtggtgggtcctgatgatctctatgatgtctactgttatgccgaagacctaaatggagaactgttcctaggcgcccctcccagcaagctgacatgggaggaggctcgggactactgtctggaacgtggtgcacagatcgctagcacaggccagctgtacgcagcctggaatggtggcctggacagatgtagccctggctggctggctgatggcagcgtgcgctatcccatcatcacacccagccaacgctgtgggggcggcctgccaggagtcaagaccctcttcctctttcccaaccagactggcttccccagcaagcagaaccgcttcaatgtctactgcttccgagactctgcccatccctctgcttcctctgaggcctctagcccagcctcagatggacttgaggccattgtcacagtgacagaaaagctggaggaactgcagctgcctcaggaagcgatggagagcgagtctcgtggggccatctactccatccccatctcagaagatgggggaggaggaagctccaccccagaagacccagcagaggcccccaggactccgctagaatcggaaacccaatccattgcaccacctaccgagtcctcagaagaggaaggcgtagccctggaggaagaagaaagattcaaagacttggaggctctggaggaagagaaggagcaggaggacctgtgggtgtggcccagagagctcagcagccctctccctactggctcagaaacagagcattcactctcccaggtgtccccaccagcccaggcagttctacagctggatgcgtcaccttctcctgggcctccaaggttccgtggaccgcctgcagagactttgctccccccgagggagtggagcgccacatctactcctggtggggcaagagaagtagggggggaaactgggagccctgagctctctggggttcctcgagagagcgaggaggcagggagctccagcttggaggatggcccttccctacttccagctacatgggcccctgtgggtcccagggagctggagaccccctcagaagagaagtctggaagaactgtcctggcaggcacctcagtgcaggcccagccagtgctgcccaccgacagtgccagccacggtggagtggctgtggctccctcatcaggtgactgtatccccagcccctgccacaatggtgggacatgcttggaggagaaggagggtttccgctgcctatgtttgccaggctatgggggggacctgtgcgatgttggccttcatttctgcagccctggctgggaggccttccagggagcctgctacaagcacttttccacacgaaggagttgggaggaggcagaaagtcagtgccgagcgctaggtgctcatctgaccagcatctgcacccctgaggagcaagactttgtcaatgatcgataccgggagtaccagtggattgggctcaatgacaggaccatcgagggtgacttcttgtggtcagatggtgcccctctgctctatgaaaactggaaccctgggcagcctgacagctacttcctgtctggggagaactgtgtggtcatggtgtggcatgaccagggacagtggagtgatgtgccctgcaactaccatctatcctacacctgcaagatggggcttgtgtcctgtgggcctccaccacagctacccctggctcaaatatttggtcgccctcggctgcgctacgcggtggatactgtgcttcgatatcgatgccgagacgggctggctcagcgcaacctgccgttgatccgctgccaggagaatgggctttgggaggcccctcagatttcctgtgtaccccggaggcctggccgtgctctgcgctccatggacgccccagaaggaccacggggacagctctcgaggcacaggaaggcaccgttgacaccgccctccagtctctagggagcctggaagactgctgcccccagcaggaccctctcacatcaactgccagtgctcttccccatgatagggggtgacgtgagaggggtgggactgaaattcagaggacagcgctcgaaggggtttctgggaaacacttgggtggctccgccccctcacacaagggcctcaggttttacccggtaagtccctaagtgcctcaactgccctctcatgtcagctgcctccttgtccctcgatntcgtnaggggacactgtgctattcgatcttgattgtcgaagagtttttaggatggagtaccagcaaaaccaggtggaaataaagttgtctgaacccaaagaaaaaaaaaa

SEQ ID NO: 8 - protein, 883 aa, Mus musculus
MIPLLLSLLAALVLTQAPAALADDLKEDSSEDRAFRVRIGAAQLRGVLGGALAIPCHVHHLRPPRSRRAAPGFPRVKWTFLSGDREVEVLVARGLRVKVNEAYRFRVALPAYPASLTDVSLVLSELRPNDSGVYRCEVQHGIDDSSDAVEVKVKGVVFLYREGSARYAFSFAGAQEACARIGARIATPEQLYAAYLGGYEQCDAGWLSDQTVRYPIQNPREACSGDMDGYPGVRNYGVVGPDDLYDVYCYAEDLNGELFLGAPPSKLTWEEARDYCLERGAQIASTGQLYAAWNGGLDRCSPGWLADGSVRYPIITPSQRCGGGLPGVKTLFLFPNQTGFPSKQNRFNVYCFRDSAHPSASSEASSPASDGLEAIVTVTEKLEELQLPQEAMESESRGAIYSIPISEDGGGGSSTPEDPAEAPRTPLESETQSIAPPTESSEEEGVALEEEERFKDLEALEEEKEQEDLWVWPRELSSPLPTGSETEHSLSQVSPPAQAVLQLDASPSPGPPRFRGPPAETLLPPREWSATSTPGGAREVGGETGSPELSGVPRESEEAGSSSLEDGPSLLPATWAPVGPRELETPSEEKSGRTVLAGTSVQAQPVLPTDSASHGGVAVAPSSGDCIPSPCHNGGTCLEEKEGFRCLCLPGYGGDLCDVGLHFCSPGWEAFQGACYKHFSTRRSWEEAESQCRALGAHLTSICTPEEQDFVNDRYREYQWIGLNDRTIEGDFLWSDGAPLLYENWNPGQPDSYFLSGENCVVMVWHDQGQWSDVPCNYHLSYTCKMGLVSCGPPPQLPLAQIFGRPRLRYAVDTVLRYRCRDGLAQRNLPLIRCQENGLWEAPQISCVPRRPGRALRSMDAPEGPRGQLSRHRKAPLTPPSSL

SEQ ID NO: 9 - DNA, 3977 bp, Mus musculus
  CDS (232)..(2784)
gaattcgcggccgcgtcgaccggatggcgcagttgaactgcgctgctgagctcgcggccgcctgcgcgcgctggggggactcgtctgggctactcccccacctcgcgttggacgaccggtcctggacacaccgcttgcgaggcaagttgaacattgcagagaaggatcttaaggctacacccgacttgccacacttgccatccagctgaagaaccaaaggctgttggagagatggcagtgacatcccaccacatggtccctgtgtttgtcctgatgagcgcctgcctggccaccgcaggtccagagcccagcacccggtgtgaactgtcaccgatcagtgcctctcatccagtccaggccctgatggagagcttcaccgttctgtctggctgtgccagcagaggcaccactgggctgccaagggaggttcacatcctaaacctccgcagtacagaccaaggactaggccagccgcagagagaggttaccctgcatctgaaccccattgcctccgtgcacactcaccacaagcctgttgtgttcctgctcaactccccacagcccctggtgtggcatgtgaagacagagagactggctgctggtgtccccagactcttcctggtttcggagggttctgtggtccagttttcatcaggaaacttctccttgacagcagaaacagaggaaaggagtttccctcaagaaaatgagcatctgctacactgggcccaaaaggaatatggagcagtgacttcattcaccgaacttaagatagcaagaaatatctatattaaagtgggagaagatcaagtgttccctcccacgtgtaacatagggaaaaattttctctcgctcaattaccttgcggagtaccttcaacccaaagccgccgaaggttgtgtcctggccagtcagccccacgagaaggaagtgcatatcattgagttaatctcccccaactccaatccttacagcaccttccaggtggatataataattgacatacgacctgctcgagaggatcctgaggtggtcaaaaacctcgtcctgatcttgaagtgcaaaaaatctgtcaactgggtgatcaagtcttttgacgtcaagggaaacttgaaagttattgctcctgacagtattggctttggaaaagagagtgaacgatccatgacagtgaccaaattggtaagaaatgactacccttccacccaagagaatctgatgaagtgggcattggacaatggctacagcccagtgacgtcatacaccatagctcctgtggccaatagatttcatcttcggcttgagaacaacgaggagatgagagatgaggaagtccacaccattcctcctgagcttcggatcctgctgggccctgaccacctgcctgccctggacagcccacccttccaaggggaaatcccaaatggaggtttcccctttccattcccggatatccccaggagaggctggaaggagggagaagataggatcccccggccaaaggaacccatcattcccagagttcaattgcttccagaccacagagagccagaagaagtgcaagggggcgtgaatatcgccctgtcagtcaaatgtgacaatgaaaagatggtggtagctgtagacaaagattctttccagaccaatggctactcggggatggagctcaccctgttggatccttcctgcaaagccaagatgaatggtacccactttgttctggagtctccgctgaatggctgtggtactagacatcggaggtcagccccagatggtgtggtttactataactctattgtggtgcaggctccatcccctggggatagcagtggctggccagacggctacgaagatttggagtcgggtgataatggatttcctggagacacagatgaaggagaaactgcccccctgagccgtgctggagtggtagtgtttaactgcagcttgcggcagctgaggagtcccagtggcttccaggaccagctcgatggaaatgctaccttcaatatggagctgtataacacagacctctttctggtgccctccccgggggtcttctctgtggcagagaatgagcatgtatatgttgaggtgtctgtcactaaggctgaccaagatctgggatttgccatccaaacctgctttatctctccatactcaaacccagacagaatgtctgattacaccatcatcgagaacatctgtccgaaggatgactctgtgaagttctacagctccaagagagtgcacttccccatcccacatgctgaagtggacaagaagcggttcagctttgtgttcaagtccgtgttcaacacctccctgctcttcctgcactgcgagctgacgctgtgctctaggaacaagggctcccagaagctgccaaagtgtgtgactcctgatgacgcctgcacctctctagatgccaccatgatctggaccatgatgcagaataagaagacattcaccaagcccctggccgtggtcctccaagtagattataaagaaaatgttccaaacatgaaggagtccagtccggttcctcctcctccacagattttccacggcctggacacgctcaccgtgatgggcattgcgtttgcagcatttgtgatcggagcactcctgacgggggccttgtggtatatctactcccacacaggggagacggcacgaaggcagcaagtccctacctcgccaccagcctcggagaacagcagcgcagcccacagcatagggagcactcagagcactccctgctctagcagcagcacggcctaggtggacagatggacgccgctgcctaggtggacagacggatgcctgccaccgccggggcagggccagatgccgatgctgggtgtccagactcagaaggcttgatctgggttccctgtaaagagagagtgaatttcagtatacagacagcctgctctacccaccccttccttaccacggcccacgtaaatgtgaccctggacatctgtcacacgaaagctaagctggtggccttccctgccagcccctcgcaggatgggggtttcaatgtgaaacatatgccagtttttgttctttttttttttttattttttatgctgctttgtccaggtgtccaaacatccatcatttggggtcgtatgttttaaagagtaaaggagacggtgaagggatgtcagctagagcgtagagccagggagagacagccagggattctcgcctagctgaaccaagatgtaaaataggagaaatgctccctccatagccaatctgcatctcgctctggatgcccactcctgacctgccacgccctacatggtcccctgtgtagcttgctagccttgggggcctgtcacttgccaaagtaacattcctctcttgggccaaaatgaatgctgactcctgatactccgcacaagcacaccaactccaaaagtgccttaaagccaggtttgtgtctcctttcaggagagagcgtgtaacgtcagatttgtgcctgtctcgaacattctgtcctaatcatacttaaacatcaggcaggtggggttcagtggtaagcgaagggattattagcaaggtaatgcctgtgtatggtcatataggcacaaatgcagaacacccagcacattggagagacgaccacttcctcctttaaaatgtcattgcttccagagtcggaacctgagttatatgtggtagattatattgactccaaccatcagcctttctgatgagcctttagagaagcctagagagcccgggcatctgatgatgtcacagtgctgcctcagatgatacagagacagggagtttcaggtgaacttcttcagcagaataaggtgatgccactctgacctttgctgagctcagtgactcttcagtgttaaaactgaacccagaggggattctccatgccagatccgctgcaaattaaataataccaattttctagagtccgtcgacgcggccgcgaattc

SEQ ID NO: 10 - protein, 850 aa, Mus musculus
MAVTSHHMVPVFVLMSACLATAGPEPSTRCELSPISASHPVQALMESFTVLSGCASRGTTGLPREVHILNLRSTDQGLGQPQREVTLHLNPIASVHTHHKPVVFLLNSPQPLVWHVKTERLAAGVPRLFLVSEGSVVQFSSGNFSLTAETEERSFPQENEHLLHWAQKEYGAVTSFTELKIARNIYIKVGEDQVFPPTCNIGKNFLSLNYLAEYLQPKAAEGCVLASQPHEKEVHIIELISPNSNPYSTFQVDIIIDIRPAREDPEVVKNLVLILKCKKSVNWVIKSFDVKGNLKVIAPDSIGFGKESERSMTVTKLVRNDYPSTQENLMKWALDNGYSPVTSYTIAPVANRFHLRLENNEEMRDEEVHTIPPELRILLGPDHLPALDSPPFQGEIPNGGFPFPFPDIPRRGWKEGEDRIPRPKEPIIPRVQLLPDHREPEEVQGGVNIALSVKCDNEKMVVAVDKDSFQTNGYSGMELTLLDPSCKAKMNGTHFVLESPLNGCGTRHRRSAPDGVVYYNSIVVQAPSPGDSSGWPDGYEDLESGDNGFPGDTDEGETAPLSRAGVVVFNCSLRQLRSPSGFQDQLDGNATFNMELYNTDLFLVPSPGVFSVAENEHVYVEVSVTKADQDLGFAIQTCFISPYSNPDRMSDYTIIENICPKDDSVKFYSSKRVHFPIPHAEVDKKRFSFVFKSVFNTSLLFLHCELTLCSRNKGSQKLPKCVTPDDACTSLDATMIWTMMQNKKTFTKPLAVVLQVDYKENVPNMKESSPVPPPPQIFHGLDTLTVMGIAFAAFVIGALLTGALWYIYSHTGETARRQQVPTSPPASENSSAAHSIGSTQSTPCSSSSTA

SEQ ID NO: 11 - DNA, 1767 bp, Mus musculus
  CDS (207)..(1271)
gactaatgttggggtaaacacagaaagcccattataacatgataagttcattaaaagaggtcagaatatcgtgcttcagatcacagaagcggtaacgagcagcaccctgctcagaggagaagtgaggggagacaggggcaggagacccagaatctcacgtaacctttaaacttcttcagaaggtcgtgaaaatacatgagataatcatgaaggcaactctcatcttcttccttctggcacaagtctcttgggctggaccatttgaacagagaggcttatttgacttcatgctagaagatgaggcttctggcataatcccttatgaccctgacaatcccctgatatctatgtgcccctaccgatgccagtgtcatcttcgagtggtgcagtgttctgatctgggtttggacaaagtgccctgggattttccacccgacacaaccttgctagacctgcaaaacaacaaaattacagagatcaaagaaggggccttcaagaacctgaaggacttgcataccttgatccttgtcaacaacaagatcagcaaaatcagtccagaggcattcaaacctctcgtgaagttggaaaggctttacctgtctaagaaccaactaaaggaactgcctgaaaaaatgcccagaactctccaggaacttcgtgtccatgagaatgagatcaccaagctgcggaaatccgacttcaatggactgaacaatgtgcttgtcatagaactgggcggcaacccactgaaaaactctgggattgaaaacggagccttccagggactgaagagtctctcatacattcgcatctcagacaccaacataactgcgatccctcaaggtctgcctacttctctcactgaagtgcatctagatggcaacaagatcaccaaggttgatgcacccagcctgaaaggactgattaatttgtctaaactgggattgagcttcaacagcatcaccgttatggagaatggcagtctggccaatgttcctcatctgagggaactccacttggacaacaacaaactcctcagggtgcctgctgggctggcacagcataagtatatccaggtcgtctaccttcacaacaacaacatctccgcagttgggcaaaatgacttctgccgagctggacacccctctcgaaaggcttcctactcggctgtgagtctttacggcaaccctgtccggtattgggaaatctttccaaacaccttcagatgtgtctatgtgcgttctgccattcaacttggaaactacaagtaaccctcagacggcctaattcttataatctggaaaaacaccccaatatgtcaatatcattgctaaaaagaaaaaatactaaaaaagaaagaaagaatgctagattctgaaaattcaagcacactgtgcatgccttttccacatgacttattatgcaagctgaatatacagattgaataattgcctacaataaaaatattttacttgtagtattcagaatcacttttaaagttgtctgtagttgtgaactgagttatcaaagtctgatgtaatcataaatgtcaaccacttagtaaagcgttaaaaggaacagaatacaaagtctctgtaattcatagagctaaattaactcttttgacataaagtcaaatgccgccgaactctagcaatgtattaatctcctttattattggtgaagccttatttgagtatgtaagttcaatatgggctatgtaaaattttcactgtcatattgaaaaaaataaaattttaaaaatgc

SEQ ID NO: 12 - protein, 354 aa, Mus musculus
MKATLIFFLLAQVSWAGPFEQRGLFDFMLEDEASGIIPYDPDNPLISMCPYRCQCHLRVVQCSDLGLDKVPWDFPPDTTLLDLQNNKITEIKEGAFKNLKDLHTLILVNNKISKISPEAFKPLVKLERLYLSKNQLKELPEKMPRTLQELRVHENEITKLRKSDFNGLNNVLVIELGGNPLKNSGIENGAFQGLKSLSYIRISDTNITAIPQGLPTSLTEVHLDGNKITKVDAPSLKGLINLSKLGLSFNSITVMENGSLANVPHLRELHLDNNKLLRVPAGLAQHKYIQVVYLHNNNISAVGQNDFCRAGHPSRKASYSAVSLYGNPVRYWEIFPNTFRCVYVRSAIQLGNYK

SEQ ID NO: 13 - DNA, 1474 bp, Mus musculus
  CDS (152)..(556)
gcctctgtctccctctctccacgaactgcccaggagcgagcagctgctcccggttggccctgacggacagacaaaccgacagcctgacaacctagtccaccaactaagcagcctgcacctggctgcttgtccctccccaggaacattgaccatgtgtcccctgtggctactcaccttgctgctggccctgagccaggccttgccctttgagcagaagggtttctgggacttcaccttggatgatgggctgctcatgatgaatgatgaggaggcttcaggttcagacaccacttcaggtgtccccgacctggactctgtcacacctaccttcagtgccatgtgtcctttcggttgccactgccacctgcgggttgttcagtgctctgacttgggtctgaagactgtgcccaaggagatctcacctgacaccacactgctagacctgcagaacaatgacatttctgagcttcgcaaggatgacttcaaaggcctccagcacctctacgtaagcctcctcctgcctcctgccttttctgaggagccaggaggagtttgatgttcctatgacttggaagaagggtactcacaaatgaggttgggaagaggggtgggaaccgatggagtcctgagataagtctggagctggctgtaatgagaacgcccaaaaccatgcattatatgtatgggtacaagacgaaatctgtcaggtcccatttttttttgacagaaatctttacgttttgttgactcatctgaacaacacagaaagtgtattccatcatagctcttagagaaactgtctagcaaagtatgctaggcagtgatggcattaccagggcagcttggagctaacctcagctccttgccctacactctactcaccaacacatactgatctccatgagaccattaaaggttcagatgtttcccaagtgccatcagtagcaagttcaggaggcagaggagaggggaggtggagtggggggctggggtgctcaccctggttgcctgccccgttttaggccctggtcttggtaaacaataagatctccaagatccatgagaaggcctttagccctctgcggaagctgcaaaaactctacatctccaagaaccacctggtggagattcctcccaacctgcccagctccctggtagaactacgaatccatgacaaccgtatccgcaaagtgcccaagggcgtgttcagcgggctccggaacatgaactgcattggtgagactagtctgtaccttagcatcaggcataaaggacacaggtagaggatatctctttcagccagggcagctgggtccatgtagtcatggaaaagccctgtgtgcctcagaatgttccagatgtttgttttgggacatgaagaaaaagactgtggtggctataaaggctcggttgaagaaagagtctcaaatatagtctaaaaaagaccagtatgtttctgaaaaaaaaaaaaaaa

SEQ ID NO: 14 - protein, 134 aa, Mus musculus
MCPLWLLTLLLALSQALPFEQKGFWDFTLDDGLLMMNDEEASGSDTTSGVPDLDSVTPTFSAMCPFGCHCHLRVVQCSDLGLKTVPKEISPDTTLLDLQNNDISELRKDDFKGLQHLYVSLLLPPAFSEEPGGV

SEQ ID NO: 15 - DNA, 2860 bp, Mus musculus
  CDS (78)..(1208)
gctgccacattctccaacccaaggagaccagacagaaggacgtggtcactctgaacagttcaacccaagagacaaaaatgcagtgggcctccgtcctgctgctggctgggctctgctcactttcccagggccagtatgatgaagactctcactggtggatccaatacctccgaaaccagcagtccacctactacgacccctatgacccttacccctatgagccctctgagccttacccctatggagtggaagaaggcccagcctatgcctatggtgcaccacctccaccagagccccgtgattgtccccaagaatgcgactgtccccccaacttccccacagccatgtactgtgacaaccgcaacctcaagtacctgccttttgtgccctcccgcatgaagtacgtctacttccagaacaaccagatctctgccatccaggaaggtgtctttgacaatgccactgggctcctctgggtcgctctacatggcaaccagattaccagtgacaaggtaggcaggaaagtcttctccaagctgaggcacttggagaggctgtacttggaccacaacaacctgacccggatgcccggcccgctgccccgatccctgagagagctccacctggaccacaaccagatctcacgggtccccaacaatgctttggagggcctggagaacctcacggccttatatctccaccacaatgagatccaggaagtggggagttccatgagaggcctccggtccctgatcctactagacctgagttataaccaccttcggagggtacccgacggtctgccctcagccctggagcagctgtacctagaacacaacaatgtctacaccgtccctgacagctacttccgggggtcacccaagctgctgtacgtccggttgtctcacaacagtctcactaacaacggccttgctaccaacaccttcaactccagcagccttcttgagctagacttgtcttacaatcagctgcagaagatccctcctgtcaacaccaacctggagaatctttacctccagggcaacaggatcaatgagttctccatcagcagcttctgcacggtggtggacgtcatgaacttctccaagctgcaggtgctacgcctggatgggaacgagatcaagcgcagcgcaatgccggtggacgccccactctgcctgcgcctcgccaacctcatcgagatctgaggggcctcgccttgggctcctggctccgggctccgagtaccgtgcaccgtgcaccgggctcggggtacatgccctgccaccccgctgcatgtttggcttttgctggatggtctgggacacgcatgtgacagaagtccacaggatcttattcaatctccttccaacaggcagagttaggtgggatcaggggccaggccagtttctgcagggggatgaatttggtggtaaaagaaatatagctatagagtctagccccaaaatcttcttgcttggacagtagcatataaccagttccaatttgaccttttttgagctgtgctcaacagcatggccagctgcttctgcagttgctctgggctcctgctctttgctcctcaaaacatcacaacacacctcttgcccagtcacctcctcccagccccagctcaccctctctgccctctttcactggggcctcttctaatgtcttaggcagtcaggagacacccacaccctaagggcacactcttatctgaggctaaacggatggctaaaatcacacacttacccccacctcacctgtttaaagtcaccatattgaacaccataatgatgagctgttaacataggggataaccgactctataatggaggatctatcagaggagggaagtgtccagtggtcattatcagtatccatagtaagatggggaagagacacacctcaggatagcagaactgaaggggcagccccctttccagtttcatctgacacaacatgatctgcaccccttcttgggcttttagtatcgaaggggcaagcgtggttttcaaaacatgagaaagagcctctgctcatcctttggtctagtatgaagggttgttacgcaaatggcagaggcagactccacccgccgaggacctacccactaacccagactagggggtgatctcaatagctcgtttgtccataatgctagagcatcaggaaccagccctgtggagatgctcactggggtgcaagaagccctgccaggcccaagtcctgccgacactagacctggcttcctctgcattcacactatgcagtccgccttatgcccagtggtgcctgtgatttgtagtttgtctaagaacagccccatcttctcctttttaaaggtgatattgcctaggtacaaccaacagcttcttctagaccccactgggatagagccaccagggtagcgagtggcaagaagcagttgtaagatgctagcctgaggtgggtccctgccttagacccctgactctgtgtgcacttgggagcaagcttctctggaaaggacagggtggggcggggtgcttagactgtgctacgggttccagaactgcaatccacaaaagccaaaccagcttgtttcaaccggggagtgccacctgcggagcaaggctgccctggccagggttcttgggaagcacctgcatgtatcaccatcctgcctctgaagagccctcagcatgaagcaagcatgaatggcggccaacctaaccaataaagttattttataagtgcatctgcaaggatggaatggttggtggcagccctcggcatcgcctcggggtacctagacacacaggccttgaaccgtgtcagctctttataaagagtagcctatataaaaactcaagtt

SEQ ID NO: 16 - protein, 376 aa, Mus musculus
MQWASVLLLAGLCSLSQGQYDEDSHWWIQYLRNQQSTYYDPYDPYPYEPSEPYPYGVEEGPAYAYGAPPPPEPRDCPQECDCPPNFPTAMYCDNRNLKYLPFVPSRMKYVYFQNNQISAIQEGVFDNATGLLWVALHGNQITSDKVGRKVFSKLRHLERLYLDHNNLTRMPGPLPRSLRELHLDHNQISRVPNNALEGLENLTALYLHHNEIQEVGSSMRGLRSLILLDLSYNHLRRVPDGLPSALEQLYLEHNNVYTVPDSYFRGSPKLLYVRLSHNSLTNNGLATNTFNSSSLLELDLSYNQLQKIPPVNTNLENLYLQGNRINEFSISSFCTVVDVMNFSKLQVLRLDGNEIKRSAMPVDAPLCLRLANLIEI

SEQ ID NO: 17 - DNA, 1723 bp, Mus musculus
  CDS (125)..(1093)
gacagttggaagatgacagttgaccagaattcctatccattggtcaggggcaaataccaaggactctgtatgctcagtcactctgtgcgacttcatcaagtcggagccaaagaaatcttgaaagatgggaatgctagcaagagttgccctgggacttatcatcattgatgctgtattggctgcaccaactacagagctctttaactatgactcagaagtgtatgatgccatcttggaggacacagggaccttttataactatgaacacattcctgacaaccatgtagagaacgagaaagtgtctgagaggctttctgggaacagagagctcctgaccccaggcccacagctaggggacaaccaggatgaagacaaggatgaagaatctactcccaggctgatcgatggctcttccccacaggagccagaattcccggggcttctgggtccacacaccaacgaagactttccaacttgtctcctgtgtacttgtataagtaccactgtatactgtgatgaccatgaactggacgctattcctccactgcccaagaagactacatatttctattcacgttttaacagaattaaaaagatcaacaaaaatgactttgcaagcttaaatgatttaaaaaggatcgatctgacatcaaatttaatatcggagattgatgaagatgcattccgaaagctgcctcacctccaagagctggttctgcgtgacaataagataaagcagcttccagaattgccaaacactttgacatttattgatatcagcaacaatagactgggaagaaaaggcattaaacaagaagcttttaaagatatgtatgatctccaccatctctatatcaccgataacagcttggaccacatccctctaccactcccagaaagtctgcgggctcttcacctccagaataatgacattctggagatgcatgaagatactttctgcaatgttaaaaatttaacttatgttcgtaaggcattagaggatattcgactggatggaaaccctatcaacctcagcagaactcctcaagcctacatgtgtctacctcgtttgcccattgggagttttatctaatgttaggatgctaggcaaatatcagaattgtgatgctttctgtagccaagagaagcatatatctcttcagaagcaaactcagtatatttgagatgcgtttaaaacatgtggcacaatgatataaacaataagctaaaggtgttgagataaaataagagttcaataatttaattaaacatgattaggtataaattaacaaaatatatagacagtaagttaaataaaatgtatgccaactagtcatcaaatctcatcaagaattgtaggtgttcatgtcaagttaaatatgtaagaaataaaatattggtaatgaatactttagatacgttgctaagatgtagacattttaattaaacttctctagtctttattttcactcatgtatgtttcccacctgattcagttaagagcagaaaaaaatattaaatgtgtttgaaagatatatggtcttgtgaaaaagtatttcttataattgtagtgtgcaaataactatgtaatagcagtatacattccttcagtttctaatgacatctatcatctataagaacatcaactcattttgcaaacttggttagttgacagctacctgtattcctctacatacaaaaaataaaatatgagatatagagc

SEQ ID NO: 18 - protein, 322 aa, Mus musculus
MGMLARVALGLIIIDAVLAAPTTELFNYDSEVYDAILEDTGTFYNYEHIPDNHVENEKVSERLSGNRELLTPGPQLGDNQDEDKDEESTPRLIDGSSPQEPEFPGLLGPHTNEDFPTCLLCTCISTTVYCDDHELDAIPPLPKKTTYFYSRFNRIKKINKNDFASLNDLKRIDLTSNLISEIDEDAFRKLPHLQELVLRDNKIKQLPELPNTLTFIDISNNRLGRKGIKQEAFKDMYDLHHLYITDNSLDHIPLPLPESLRALHLQNNDILEMHEDTFCNVKNLTYVRKALEDIRLDGNPINLSRTPQAYMCLPRLPIGSFI

SEQ ID NO: 19 - DNA, 4909 bp, Mus musculus
  CDS (390)..(3296)
gcagctccgagctaggtgctatcgcaaggccagagcgcacagcccggcggagagagcagatccttgctcagatcgagtcaaatcggggccaaggcggaggacgaagagtccaggctcctattctggacttgttccccagctccgggggcgcttctaggtcctgcagcagccaggagtgcggagccaccaactcggtgctggaatgaaaaaattcccgcgcgccagtgcagaatctttctaagtgacccggagcttcgggtgctagctctgcacgaactttcccatcaaagtgatcgtgaattttaagcatcaggagcaggccagcgaagctctacgcgtctaaacgtctatccagaccaagagttctctgcggtgcagggtgcggtgccatgcagccaaaagtccctttggggtcacgcaagcagaagccctgctccgacatgggggacgtccagcgggcagcgagatctcggggctctctgtccgcacacatgctgttgctgctcctcgcttccataacaatgctgctatgtgcgcggggcgcacacgggcgccccacggaggaagatgaggagctggtcctgccctcgctggagcgcgccccgggccacgattccaccaccacacgccttcgtctggacgcctttggccagcagctacatctgaagttgcagccggacagcggtttcttggcgcctggcttcaccctgcagactgtggggcgcagtcccgggtccgaggcacaacatctggaccccaccggggacctggctcactgcttctactctggcacggtgaacggtgatcccggctctgccgcagccctcagcctctgtgaaggtgtgcgtggtgccttctacctacaaggagaggagttcttcattcagccagcgcctggagtggccaccgagcgcctggcccctgccgtgcccgaggaggagtcatccgcacggccgcagttccacatcctgaggcgaaggcggcggggcagtggcggcgccaagtgcggcgtcatggacgacgagaccctgccaaccagcgactcgcgacccgagagccagaacacccggaaccagtggcctgtgcgggaccccacgcctcaggacgcgggaaagccatcaggaccaggaagcataaggaagaagcgatttgtgtccagcccccgttatgtggaaaccatgctcgtggctgaccagtccatggccgacttccacggcagcggtctaaagcattaccttctaaccctgttctcggtggcagccaggttttacaagcatcccagcattaggaattcaattagcctggtggtggtgaagatcttggtcatatatgaggagcagaagggaccagaagttacctccaatgcagctctcacccttcggaatttctgcaactggcagaaacaacacaacagccccagtgaccgggatccagagcactatgacactgcaattctgttcaccagacaggatttatgtggctcccacacgtgtgacactctcgggatggcagatgttggaactgtatgtgaccccagcaggagctgctcagtcatagaagatgatggtttgcaagccgccttcaccacagcccacgaattgggccatgtgtttaacatgccgcacgatgatgctaagcactgtgccagcttgaatggtgtgactggcgattctcatctgatggcctcgatgctctccagcttagaccatagccagccctggtcaccttgcagtgcctacatggtcacgtccttcctagataatggacacggggaatgtttgatggacaagccccagaatccaatcaagctcccttctgatcttcccggtaccttgtacgatgccaaccgccagtgtcagtttacattcggagaggaatccaagcactgccctgatgcagccagcacatgtactaccctgtggtgcactggcacctccggtggcttactggtgtgccaaacaaaacacttcccttgggcagatggcaccagctgtggagaagggaagtggtgtgtcagtggcaagtgcgtgaacaagacagacatgaagcattttgctactcctgttcatggaagctggggaccatggggaccgtggggagactgctcaagaacctgtggtggtggagttcaatacacaatgagagaatgtgacaacccagtcccaaagaacggagggaagtactgtgaaggcaaacgagtccgctacaggtcctgtaacatcgaggactgtccagacaataacggaaaaacgttcagagaggagcagtgcgaggcgcacaatgagttttccaaagcttcctttgggaatgagcccactgtagagtggacacccaagtacgccggcgtctcgccaaaggacaggtgcaagctcacctgtgaagccaaaggcattggctactttttcgtcttacagcccaaggttgtagatggcactccctgtagtccagactctacctctgtctgtgtgcaagggcagtgtgtgaaagctggctgtgatcgcatcatagactccaaaaagaagtttgataagtgtggcgtttgtggaggaaacggttccacatgcaagaagatgtcaggaatagtcactagtacaagacctgggtatcatgacattgtcacaattcctgctggagccaccaacattgaagtgaaacatcggaatcaaagggggtccagaaacaatggcagctttctggctattagagccgctgatggtacctatattctgaatggaaacttcactctgtccacactagagcaagacctcacctacaaaggtactgtcttaaggtacagtggttcctcggctgcgctggagagaatccgcagctttagtccactcaaagaacccttaaccatccaggttcttatggtaggccatgctctccgacccaaaattaaattcacctactttatgaagaagaagacagagtcattcaacgccattcccacattttctgagtgggtgattgaagagtggggggagtgctccaagacatgcggctcaggttggcagagaagagtagtgcagtgcagagacattaatggacaccctgcttccgaatgtgcaaaggaagtgaagccagccagtaccagaccttgtgcagaccttccttgcccacactggcaggtgggggattggtcaccatgttccaaaacttgcgggaagggttacaagaagagaaccttgaaatgtgtgtcccacgatgggggcgtgttatcaaatgagagctgtgatcctttgaagaagccaaagcattacattgacttttgcacactgacacagtgcagttaagaggcgttagaggacaaggtagcgtggggagggctgatacactgagtgctggagggatccagtgagtcaaaccagtaagcagtgaggtgtggcaaggaggtgtgtgtaggggatacatagcaaaggaggtagatcaggacactaccctgccagttacattctgataaggtagttaatgaggcacagtagcatctgaaagaccatacagagcactaaggagccccaaagcactattagtatctcttttcttatatctatcgcccaaataattttcagagtctggcagaagccctgttgcactgtactgactagatacttcttatcacaaagattgggaaaggcaaagcagaaagatggtaagactgggtttcaaacaaggcttggtttctatcactggaggcaaggaggaggggacaaacaagatcattattcgaagtcgctggttgctgtggttttacggaaggttgatgcatcattcctatcaacagtgaaaagttcagcttgttcaacgtgacagaaaggctcatctccgtgaaagagctcctgatttcttcttacaccatctcagttcttaactataattcatgttgaggtagaaacaattcatctatttataaaatgtacattggaaaaaaaaaagtgaagtttatgaggtacacataaaaactgaaggaaacaatgagcaacatgcctcctgctttgcttcctcctgaggtaaacctgcctggcgattgaggttgtttaagattatccatggctcacaagaggcagtaaaataatacatgttgtgccagagttagaatggggtatagagatcagggtcccatgagatggggaacatggtgatcactcatctcacatgggaggctgctgcagggtagcaggtccactcctggcagctggtccaacagtcgtatcctggtgaatgtctgttcagctcttctactgagagagaatatgactgtttccatatgtatatgtatatagtaaaatatgttactatgaattgcatgtactttataagtattggtgtgtctgttccttctaagaaggactatagtttataataaatgcctataataacatatttatttttatacatttatttctaatgataaaacctttaagttatatcgcttttgtaaaagtgcatataaaaatagagtatttatacaatatatgttaactagaaataataaaagaacacttttgaatgtgtatgcctattttctggagtgggattaacttctgggcaagaaatctgatgagacacaaacattggacttcaagacagttttaaattttgggtaaatgaactgtatttcctgtttatagacgtactaataaaaaagaagttgatgatgtctttagtggtaagattgttactaatgtggttggcaaattgctgtaaagagccagatagtaagcatttatggcattgtaggctatctttcctgccacaaccatgtgacagtgagtgctttgtaggactgagagcagccataaatgacatgtaaatgataaactgtggctgtgctttaataaaactttatttacaaaaacaaaaaaaaaaaaaaaaaaaaaaaaaaaaaaaaaaaaaaaaaaaaaaaaaa

SEQ ID NO: 20 - protein, 968 aa, Mus musculus
MQPKVPLGSRKQKPCSDMGDVQRAARSRGSLSAHMLLLLLASITMLLCARGAHGRPTEEDEELVLPSLERAPGHDSTTTRLRLDAFGQQLHLKLQPDSGFLAPGFTLQTVGRSPGSEAQHLDPTGDLAHCFYSGTVNGDPGSAAALSLCEGVRGAFYLQGEEFFIQPAPGVATERLAPAVPEEESSARPQFHILRRRRRGSGGAKCGVMDDETLPTSDSRPESQNTRNQWPVRDPTPQDAGKPSGPGSIRKKRFVSSPRYVETMLVADQSMADFHGSGLKHYLLTLFSVAARFYKHPSIRNSISLVVVKILVIYEEQKGPEVTSNAALTLRNFCNWQKQHNSPSDRDPEHYDTAILFTRQDLCGSHTCDTLGMADVGTVCDPSRSCSVIEDDGLQAAFTTAHELGHVFNMPHDDAKHCASLNGVTGDSHLMASMLSSLDHSQPWSPCSAYMVTSFLDNGHGECLMDKPQNPIKLPSDLPGTLYDANRQCQFTFGEESKHCPDAASTCTTLWCTGTSGGLLVCQTKHFPWADGTSCGEGKWCVSGKCVNKTDMKHFATPVHGSWGPWGPWGDCSRTCGGGVQYTMRECDNPVPKNGGKYCEGKRVRYRSCNIEDCPDNNGKTFREEQCEAHNEFSKASFGNEPTVEWTPKYAGVSPKDRCKLTCEAKGIGYFFVLQPKVVDGTPCSPDSTSVCVQGQCVKAGCDRIIDSKKKFDKCGVCGGNGSTCKKMSGIVTSTRPGYHDIVTIPAGATNIEVKHRNQRGSRNNGSFLAIRAADGTYILNGNFTLSTLEQDLTYKGTVLRYSGSSAALERIRSFSPLKEPLTIQVLMVGHALRPKIKFTYFMKKKTESFNAIPTFSEWVIEEWGECSKTCGSGWQRRVVQCRDINGHPASECAKEVKPASTRPCADLPCPHWQVGDWSPCSKTCGKGYKKRTLKCVSHDGGVLSNESCDPLKKPKHYIDFCTLTQCS

SEQ ID NO: 21 - DNA, 3673 bp, Mus musculus
  CDS (392)..(2929)
ggggagaacccggggaagacccacagatacacagaaacgagagagacagaagaggagagagacagagacaaagagacagcagcaggaggaggaggaggaggaggcaaagacagggcgagcactgaggcaaagccagacagctgagcagagggagaagccaaggagtcacagacaaagacctgggacagaaaggcaaaggaggaagttttggagcagaaagaacccgtgggcacttttcctgagcccaggggctgttttctttttcatcttctctgaaagtccctagcccatttaaaaactttgcctcagattttggcacaagccccaaagcccttctggctttaacgaggagccttccacagttagggtgtggagcattttggtgccgcagatggcctcaatccatcccagctgcagcccgggtaccatgtcccagatgggcttgcatcccaggaggggcttgactgggcactggctgcaaagattccaaccctgcttgccgcttcacactgtgcagtggcggaggctgctgctgctggccttcctcctgtccttagcgtggcccgccagccccctcccccgggaggaggagatcgtgtttccagagaagctcaatggcagtagcatcctacctggatcaggcgttcctgccaggctgctgtaccgattgccagcctttggggagatgttgctactagaactagaacaggaccctggggtgcaggtagagggtttgactgtacagtacctgggccaggcacctgagatgctgggtggggcagagccaggtacctacctgactggcaccatcaatggagatccggagtcggtggcatctctgcactgggacgggggagccctattaggggtactgcagtaccgtggggccgaactccacctccagcctctggaaggaggcgcccttaactctgctgggggaccgggggctcacatcctacgccggaagagtcctgccagcagccaaggtcccatgtgcaccgtcaaggctccttctgggagcccgagtcccatttcccgcagaaccaagcgcttcgcttctctgagtagattcgtggagacactggtggtagcagatgacaagatggcagcattccatggtacagggttaaagcgctacctgctgacggttatggcagctgccgctaaagcctttaaacacccaagcatccgaaaccctgtcaacttggtggtgacgcgcctggtgatcctggggtccggccaggaagggccccaagtggggccaagtgccgcccagaccctacgcagcttctgcacctggcagcggggcctcaacacccctaacgactcagatcctgaccactttgacacagccattctgttcacccggcaggacctgtgtggggtctccacttgtgacaccctgggtatggctgatgtgggcacagtgtgtgatccagctaggagctgtgctattgtggaagatgatgggctccagtcagccttcactgctgctcatgaactgggccatgtcttcaacatgctccatgataactccaagccatgcactaacttgaatgggcaggggggttcctctcgccatgtcatggctcctgtcatggcccatgtggaccctgaagagccctggtcgccctgcagtgcccgattcatcactgacttcctggacaatggttatgggcactgcctcttagacaaaccggaggctcccctccatctaccagcgacttttcctggcaaggactatgacgctgaccgccaatgccaactgaccttcggtcctgactcaagccattgtccacagctgccaccgccctgtgctgccctctggtgctctggccacctcaatggccatgccatgtgccagacgaagcactcaccttgggctgatggcactccctgcgggtcttcacaggcctgcatgggtggccgctgtctgcacgtggaccagctcaaggacttcaatgttcctcaggctggaggctggggcccctggggaccatggggtgactgctccaggacttgtgggggtggtgtccagttctcctcccgggattgcacgaggcccgtcccccggaacggtggcaagtattgtgagggccgccggactcgcttccgctcctgcaacacggagaactgcccacacggctcagcattgaccttccgtgaagagcagtgtgctgcctacaaccaccgaaccgacctcttcaagagctttccagggcccatggactgggttccgcgctacacaggtgtggcccctcgagaccaatgcaaactcacctgccaggcccgggcactgggctactactacgtattggagccccgggtggcagatgggactccctgctccccagacacctcctctgtctgtgtccagggccgctgtatccatgctggctgtgaccggatcattggctccaaaaagaaatttgacaagtgcatggtgtgcggcggggatggctctcgctgcagcaagcagtcgggctccttcaaaaaattcaggtatggatacagcgatgtggtcacgatccctgcgggggccacccatatccttgtacggcagcagggggggtctggtctcaagagcatctacctggccctgaagctttctgacggttcttacgccctcaatggtgaatacacgctgatgccctccccaacagatgtggttcttcctggggcagtcagcttgcgctacagcggagccacagcagcctcagagacactgtctggacatgggccgctggcccagcccttgacgctgcaagtcctggtggctggcaacccacagaatgcacgtctgcggtacagtttctttgtcccgcggccagtcccttcaacaccacgccctcctccccaagactggctgcaacgcagggcagagatactgaagatccttcggaagcgtccctgggcaggccggaaataacctcactgtcccggctgccctttttgggcgccggggcctcggactcatctgggagaatgagcaggcttctgcaactgcctcctgctaaaacacagtagggaggtgtagagggtgagatctgcctgcctcactgccccaaaccgcaggctggccctgccctggcttcctgccctgggaggcagtgatgtcttggtgaatggaaaggggctaggtgacagtaccctatctactaaactgccccctctaccctgcaggtcacaggaggaatggggggaagacagggtgggtcctgggccctagttgtatttatttggtatttattcatttttatttagcaccaggaaaggggactagggtcttggggaaactcacctattatagccctaacctagctatgaaatccagggtgttggtgacaaatatgagtggtgtgtgtgtgtgtgtgtgtgtgtgtgtgtgtgtttatgtatgaggtacaacctgccctgctttcctctccctaatttttttttttttctgggaaaaggaaagtcaaaggtaggactgccttcagggagtaagggatgattgtgtttttaaattgaagtttgctatttatatgctctttttggagtcagacaaatgtgggttatattctggccccgcatctttgagcattagttttctcatgtgccaataataatcccttagaaattggttgtaaggattaaatgatgttaataaagaactagcatagagcctctc

SEQ ID NO: 22 - protein, 845 aa, Mus musculus
MASIHPSCSPGTMSQMGLHPRRGLTGHWLQRFQPCLPLHTVQWRRLLLLAFLLSLAWPASPLPREEEIVFPEKLNGSSILPGSGVPARLLYRLPAFGEMLLLELEQDPGVQVEGLTVQYLGQAPEMLGGAEPGTYLTGTINGDPESVASLHWDGGALLGVLQYRGAELHLQPLEGGALNSAGGPGAHILRRKSPASSQGPMCTVKAPSGSPSPISRRTKRFASLSRFVETLVVADDKMAAFHGTGLKRYLLTVMAAAAKAFKHPSIRNPVNLVVTRLVILGSGQEGPQVGPSAAQTLRSFCTWQRGLNTPNDSDPDHFDTAILFTRQDLCGVSTCDTLGMADVGTVCDPARSCAIVEDDGLQSAFTAAHELGHVFNMLHDNSKPCTNLNGQGGSSRHVMAPVMAHVDPEEPWSPCSARFITDFLDNGYGHCLLDKPEAPLHLPATFPGKDYDADRQCQLTFGPDSSHCPQLPPPCAALWCSGHLNGHAMCQTKHSPWADGTPCGSSQACMGGRCLHVDQLKDFNVPQAGGWGPWGPWGDCSRTCGGGVQFSSRDCTRPVPRNGGKYCEGRRTRFRSCNTENCPHGSALTFREEQCAAYNHRTDLFKSFPGPMDWVPRYTGVAPRDQCKLTCQARALGYYYVLEPRVADGTPCSPDTSSVCVQGRCIHAGCDRIIGSKKKFDKCMVCGGDGSRCSKQSGSFKKFRYGYSDVVTIPAGATHILVRQQGGSGLKSIYLALKLSDGSYALNGEYTLMPSPTDVVLPGAVSLRYSGATAASETLSGHGPLAQPLTLQVLVAGNPQNARLRYSFFVPRPVPSTPRPPPQDWLQRRAEILKILRKRPWAGRK

SEQ ID NO: 23 - DNA, 3002 bp, Mus musculus
  CDS (18)..(2810)
ccggcgggcagcgcactatgcggctcgagtgggcgtccttgttgctgctactgctgctgctgagcgcgtcctgcctgtccctggccgctgacagccccgccgcggcacctgcccaggataaaaccaggcagcctcaggctgcagcagcggccgccgagccggaccagccgcagggggaggaaacacgggagcgaggccatttacaacccttggccgggcagcgcaggagcggcgggctggtccataatatagaccaactctactctggcggtggcaaagtgggctaccttgtctacgcgggcggccggaggttcctgctggacctggagagagatgacacagtgggtgctgctggtagcatcgttactgcaggaggagggctgagcgcatcctctggccaccggggtcactgtttctacagaggcaccgtggacggcagccctcgatccctagctgtctttgacctctgcgggggtctcgatggcttctttgcagtcaagcatgcgcgctacactctaaagccactcctgcgtgggtcctgggcagagtatgaacgaatttatggggatggatcttcccgcatcctgcatgtctacaaccgcgagggctttagcttcgaggccctgccgccacgcgccagttgcgagactcctgcatccccatctgggccccaagagagcccctcggtgcacagtagatctaggagacgctcagcgctggccccgcagctgctggaccactcagctttctcgccatctgggaacgcgggacctcagacttggtggaggcgtaggcgccgttccatctccagggcccgccaggtggagctcctcttggtggctgactcgtccatggccaggatgtatgggcggggcctgcagcattacctgctgaccctggcctccatcgccaacaggctgtacagtcatgcaagcattgagaaccacatccgcctggcggtggtgaaggtggtggtgctgacggacaaggacacgagtctggaggtgagcaagaatgcggccacgaccctcaagaacttttgcaaatggcagcaccaacataaccagctaggggatgatcacgaagagcactacgatgcagccatcctgttcacccgagaggatttatgtgggcatcattcatgtgacaccctgggaatggcagacgttgggaccatatgttctccggagcgcagctgtgcagtgattgaagatgatggcctccatgcagccttcactgtggctcatgaaattgggcatctacttggcctttctcatgacgattccaaattctgtgaagagaacttcggtactacagaagacaagcgtttaatgtcttcaatccttaccagcatcgatgcatccaagccctggtccaaatgcacgtcagccaccatcacagaattcctggatgatggtcatggtaattgtttgctagacctaccacggaagcagattttgggtcccgaggaactcccaggacagacctacgatgccacccagcagtgcaacttgacatttgggcctgagtactcggtgtgccctggcatggatgtctgtgcgcggctgtggtgtgctgtggtgcgccaaggccaaatggtgtgtctgaccaagaagctgccggctgtggagggcactccctgtgggaagggaagagtctgccttcaaggcaaatgtgtggacaaaactaagaaaaaatattactcgacatcaagccatggaaattgggggtcctggggcccctggggtcagtgttctcgctcatgcgggggaggagtgcagtttgcctaccgccattgtaataaccctgcacctcgaaacagtggccgctactgcacagggaagagggccatataccgttcctgcagtgttacaccctgcccacccaatggtaaatcttttcgccatgagcagtgtgaagccaaaaatggctatcagtctgatgcaaaaggagtcaaaacatttgtagaatgggttcccaaatatgcaggtgtcctgccggcagatgtgtgcaagcttacctgcagagctaagggcacaggctactatgtggtcttttctccaaaggttacggatgggactgaatgcaggccgtacagcaactctgtgtgtgtccgaggacggtgtgtgagaactggatgtgacggcattattggctcaaagctacaatatgacaagtgtggagtgtgcggaggggataactccagttgtacaaagattatcggaaccttcaataaaaaaagcaagggttatactgacgttgtgaggatccctgaaggagcaacccacataaaagtccgacagttcaaagccaaagaccagactagattccctgcctacttagccctgaagaagaaaactggcgagtaccttatcaatggcaagtacatgatttccacttcagagaccatcatcgacatcaatggtaccgtcatgaactacagtggatggagccacagagatgattttttacatgggatgggctattcagccacaaaagaaatcctgatcgtgcagatccttgccacagacccaactaaagcgctaggcgtccgttacagcttttttgttcccaagaagaccactcaaaaagtaaactctgtcatcagccatggcagcaacaaggtgggaccacactctacacagctgcagtgggtgacaggtccatggctggcctgctccaggacctgtgacacaggctggcacactaggaccgtgcagtgccaggatggaaacaggaaattagctaaaggatgccttctctctcagaggccttctgcatttaagcaatgtctgctgaagaaatgttagcctgtggtttactctaatgcacaaaaaaacaacaggaggatcatcgcagatacagctgtggtgaagacaaggcctacccaaagcacagaaagtcatgccttcatgtcattgtcaccacgagtcgaattatgggcagaatctgctctctgcgaccaaaaggtttactctacttggtgaatgatggtaccgtga

SEQ ID NO: 24 - protein, 930 aa, Mus musculus
MRLEWASLLLLLLLLSASCLSLAADSPAAAPAQDKTRQPQAAAAAAEPDQPQGEETRERGHLQPLAGQRRSGGLVHNIDQLYSGGGKVGYLVYAGGRRFLLDLERDDTVGAAGSIVTAGGGLSASSGHRGHCFYRGTVDGSPRSLAVFDLCGGLDGFFAVKHARYTLKPLLRGSWAEYERIYGDGSSRILHVYNREGFSFEALPPRASCETPASPSGPQESPSVHSRSRRRSALAPQLLDHSAFSPSGNAGPQTWWRRRRRSISRARQVELLLVADSSMARMYGRGLQHYLLTLASIANRLYSHASIENHIRLAVVKVVVLTDKDTSLEVSKNAATTLKNFCKWQHQHNQLGDDHEEHYDAAILFTREDLCGHHSCDTLGMADVGTICSPERSCAVIEDDGLHAAFTVAHEIGHLLGLSHDDSKFCEENFGTTEDKRLMSSILTSIDASKPWSKCTSATITEFLDDGHGNCLLDLPRKQILGPEELPGQTYDATQQCNLTFGPEYSVCPGMDVCARLWCAVVRQGQMVCLTKKLPAVEGTPCGKGRVCLQGKCVDKTKKKYYSTSSHGNWGSWGPWGQCSRSCGGGVQFAYRHCNNPAPRNSGRYCTGKRAIYRSCSVTPCPPNGKSFRHEQCEAKNGYQSDAKGVKTFVEWVPKYAGVLPADVCKLTCRAKGTGYYVVFSPKVTDGTECRPYSNSVCVRGRCVRTGCDGIIGSKLQYDKCGVCGGDNSSCTKIIGTFNKKSKGYTDVVRIPEGATHIKVRQFKAKDQTRFPAYLALKKKTGEYLINGKYMISTSETIIDINGTVMNYSGWSHRDDFLHGMGYSATKEILIVQILATDPTKALGVRYSFFVPKKTTQKVNSVISHGSNKVGPHSTQLQWVTGPWLACSRTCDTGWHTRTVQCQDGNRKLAKGCLLSQRPSAFKQCLLKKC

SEQ ID NO: 25 - DNA, 3077 bp, Mus musculus
  CDS (59)..(2200)
ccacgcgtccggccagccccagggaccgcagcaatctcccaaactccccttccccaggatgacagaggagttaatcacccctgtgtactgcaccggggtgtcagcccaagtacagaagaagcgggacaaggagctgggcttgggccgccatgaaaacgccatcaagtacctgggccaggattatgaaacgcttcgggcaagatgcctgcagagtggggtcctcttccaagacgaggccttccctccggtttctcatagcctgggcttcaaggaactgggtcctcattcctctaaaacctatggcatcaaatggaagcggcctacggaactgatgtcaaacccccagttcatcgtggatggagccacccggacagacatctgccagggagcactgggggactgttggctcctggctgccattgcctccctcaccctcaacgagactattctgcaccgagtggttccctacggccagagctttcaggatggctacgctggcatctttcatttccagctgtggcagttcggggagtgggtcgacgtggtcatagatgacttgctgcccaccaaggacgggaagctggtgttcgtgcactctgcccagggcaacgagttctggagcgcactgctggagaaagcctatgctaaagtgaatggcagctatgaggctctttcgggaggctgcacctcagaggccttcgaggactttaccggtggggtcactgagtggtacgacctgcagaaggcccccagcgacctctaccagatcattctaaaggccctggaacgaggctccttgctgggctgctccattaatatctccgatatccgtgatttagaggctattacttttaagaacctggtgaggggccatgcgtactctgtgacgggcgccaagcaggtaacttaccagggccagcgggtgaacctaatccggatgcggaacccctggggtgaagtggagtggaaaggaccctggagtgacagctcctatgagtggaacaaagtggacccctatgaacgagagcagctgagggtcaagatggaggatggggagttctggatgtcgttccgagacttcatccgtgaattcaccaaactggaaatctgcaaccttacaccggacgcccttaagagcaggaccctccggaattggaataccacattttacgagggcacctggcgtcggggaagcaccgctggaggctgcaggaactacccagctaccttctgggtaaacccccagttcaagatccggttggaggaggtggatgacgcagacgactatgacaaccgggagtcgggctgcagcttcttgctggccctcatgcagaaacaccgccgcagggagcgtcgctttggccgggacatggagaccatcggttttgcagtgtaccaggtccctcgggagctggcgggtcagcctgtgcacttgaagcgtgatttcttcctggccaacgcttctcgggcgcagtcagagcacttcatcaaccttcgggaagtcagcaaccgtatccgcctgccgcccggggagtacatagtggtgccctccaccttcgagcccaacaaagaaggcgacttcctgctgcgcttcttctcagagaagaaggctgggacccaggaactagatgaccagatccaggccaacctccctgatgagaaagttctctctgaagaggagattgatgacaacttcaagaccttgttcagcaagttggcaggggatgacatggagatcagcgtcaaggagctacagaccatcttgaacaggatcattagcaaacacaaagacctgcgcactaatggcttcagcctggagtcgtgccgcagcatggtgaacctcatggatcgagatggcaacgggaagctgggtctggtggagttcaacatcctgtggaaccgcatccgaaattacctgaccatcttccggaagtttgacctggacaagtctggcagcatgagtgcctatgagatgaggatggccatcgaggctgcaggcttcaagcttaacaagaagctgcatgaactcataatcacccgctactcggagcccgacctggccgtggactttgacaactttgtgtgctgtcttgtgcggctggagaccatgttccggtttttcaaacttctggacacagacctggatggtgttgtgacctttgatctatttaagtggctccagctgactatgtttgcctgaagtggggaatcgctccgctccagttgcctttgttcttccattcttcctctgccaagccacgcctccccgccaagccacgcctccccgccgtgccactccagactacaccagcttctggtgccttcctcagaacaggaaggaaggcacccttgtcctcctgttccccttgtcctcctgtcctttcacctccccactacctctgttcgactgctctgggcagtcacggggccctccctgcccctgtagtcagactgggcaccctgacccctgctcagggctaagaccaggaagacagctccctgctcatccctgcctcggggctggggcaggggcaggggcaggggcaggggcaggcacccctgcccctccccctcgctgctactgtccggatgtgagtcttgcctaatctgtggaggctctagcccctgtcatcccctagagctgcagacttcataaccactagctagctccctgtctgcacccgggtgttgaaggcctcacctcactgtgggagccccacagccagactcaccttcctgtgccttctgtgcagcagccaagatccctctgcaaaccaaactacacctctcctagcctgtgtgcccaccaggaacttcctggccccgagggtgctcaggccctccccatccccgacccctctccctctctttttttgtatatatactggtcattttcaagggaatgcctgagggatgagtgtactggttatggaggaactgggggtggggtagggaggtcccgtgtttcagtgcagtgagaaaataaaatgaaaggctgtaaaaaaaaaaaaaaaaaaaaaaaaaaaaaaaaaaaaaaaaaaaaaaaaaaaaaaaaaaaaaaaaaaaaaaaaaaaaaa

SEQ ID NO: 26 - protein, 713 aa, Mus musculus
MTEELITPVYCTGVSAQVQKKRDKELGLGRHENAIKYLGQDYETLRARCLQSGVLFQDEAFPPVSHSLGFKELGPHSSKTYGIKWKRPTELMSNPQFIVDGATRTDICQGALGDCWLLAAIASLTLNETILHRVVPYGQSFQDGYAGIFHFQLWQFGEWVDVVIDDLLPTKDGKLVFVHSAQGNEFWSALLEKAYAKVNGSYEALSGGCTSEAFEDFTGGVTEWYDLQKAPSDLYQIILKALERGSLLGCSINISDIRDLEAITFKNLVRGHAYSVTGAKQVTYQGQRVNLIRMRNPWGEVEWKGPWSDSSYEWNKVDPYEREQLRVKMEDGEFWMSFRDFIREFTKLEICNLTPDALKSRTLRNWNTTFYEGTWRRGSTAGGCRNYPATFWVNPQFKIRLEEVDDADDYDNRESGCSFLLALMQKHRRRERRFGRDMETIGFAVYQVPRELAGQPVHLKRDFFLANASRAQSEHFINLREVSNRIRLPPGEYIVVPSTFEPNKEGDFLLRFFSEKKAGTQELDDQIQANLPDEKVLSEEEIDDNFKTLFSKLAGDDMEISVKELQTILNRIISKHKDLRTNGFSLESCRSMVNLMDRDGNGKLGLVEFNILWNRIRNYLTIFRKFDLDKSGSMSAYEMRMAIEAAGFKLNKKLHELIITRYSEPDLAVDFDNFVCCLVRLETMFRFFKLLDTDLDGVVTFDLFKWLQLTMFA

SEQ ID NO: 27 - DNA, 2105 bp, Mus musculus
  CDS (418)..(1671)
gagccttcccggcgcgtgagccggatccggtggcaccgcggggaagagacaggaccgggcggtggcggcagagacagggggacgcacccggtgcagaagatccaataggagcacgccgccgcaacctctcccgcgcgctccggtcgccgactctacgccgatcgcccactccccgcaccttggactacaccgggaaaaagacgcagcctggcccgaactggggcggagatccttcttgcttcccaagagctcgggagggaagattctcccgccgccgagaaccccgccggactggaggaacccgtggcctggagagcgctggctgtgccagaccccagcctgatggatgtctggtgtggataatgagggaagaacgtgccttttacacccaagaggtgaccccggagcgtgccccggatgaccccacaactcggaacgatgcggctagcctgcatgttctcgtccatcctgctgtttggagctgcgggcctgctcctcttcatcagcctccaggaccctatagagctcagcccccagcaagttccaggtataaagttcagcatcaggccccagcaaccccagcatgatagccacttgaggatatccacagaaaagggcacacgagattcacccagcgggtcgccaagaggcctccagctgcaagcgcctgaccaacctcgacctcacccgaaggcagcgggatctcctttgcgcctccggcagcgcaggcggagactgctcatcaaaaagatgccagccgcagggactaaccaaggcaacaactcgtccgaaacctttatccagccgagaccccgcaccatggacagtcgttgggtcagcctgcaccagacccaacaggagcgcaagcgtgtgatgcgagaagcctgcgctaaatacagggccagcagcagccgcagagctgtcactccccgccacgtctcccgcatcttcgtggaggaccgccaccgtgtactgtactgtgaagtacccaaggcaggctgctccaactggaagagggtgctcatggtgctggcagggttagcctcatccacggcagatatccaacacaacaccgtccactatggcagcgcccttaagcgcctggatacttttgaccggcagggcatagtgcaccgcctcagtacctacaccaagatgctctttgtccgggaaccctttgagcggctggtctctgctttccgagacaagtttgagcatcctaacagctactatcatcctgtctttggcaaggctatcctggcccggtaccgcgccaacgcctcgcgggaggcactgcggactggctccggtgtgcagttccccgagttcgtccagtacctgttggatgtccaccggcccgtgggcatggacatccactgggaccatgttagccggctgtgcagcccctgcctcatcgactatgactttgtgggcaagttcgagagcatggaagacgatgccaacttcttcctgcgtctcatccatgcgcccgggaacctgactttcccgaggttcaaggacaggcactccgaggaggcgcggaccacatcgagaatcacccatcagtacttcgctcagctctcctcgctgcagcgacagcgaacctacgacttctactacatggattacctgatgttcaactactccaaacctttctcggacctgtactgagggcggggcctgctggtcaggggcggggtctgccggtcatgcccactcacctgcgcataggcggcctccggggactgagctctgaggatgtgaggccttgtggctgtggccctagggtgggccacagaggcccagacaatggaccttgacccttgtcccacacccatttcctcattgggttggctgagtttgagacggagcacgactcggatggatgctttaagaactcagctgagctatgctgagctatgctgtccagggaagcctgagacccagaagagggccccagcgtcgagggatgtcctacatccccttatcctttgccttgtaccaaaccacgtggtttgctgcttttctatgacccagggtcatctgaataaagcacatggttttcagagcaaaaaaaaaaaaaaaaaaaaaaaaaaaaaaaaaaa

SEQ ID NO: 28 - protein, 417 aa, Mus musculus
MTPQLGTMRLACMFSSILLFGAAGLLLFISLQDPIELSPQQVPGIKFSIRPQQPQHDSHLRISTEKGTRDSPSGSPRGLQLQAPDQPRPHPKAAGSPLRLRQRRRRLLIKKMPAAGTNQGNNSSETFIQPRPRTMDSRWVSLHQTQQERKRVMREACAKYRASSSRRAVTPRHVSRIFVEDRHRVLYCEVPKAGCSNWKRVLMVLAGLASSTADIQHNTVHYGSALKRLDTFDRQGIVHRLSTYTKMLFVREPFERLVSAFRDKFEHPNSYYHPVFGKAILARYRANASREALRTGSGVQFPEFVQYLLDVHRPVGMDIHWDHVSRLCSPCLIDYDFVGKFESMEDDANFFLRLIHAPGNLTFPRFKDRHSEEARTTSRITHQYFAQLSSLQRQRTYDFYYMDYLMFNYSKPFSDLY

SEQ ID NO: 29 - DNA, 1369 bp, Mus musculus
  CDS (40)..(1281)
attcagacatgaagagagacacagtggtctgaagtggtcatgaaagccaaacaagtcttcttttctgtcctgctgtttgggacagcagggcttctgctcttcatgtacttgcaagcatggattgaagaacatcatacagggaaaatagagaagaaaagggatcagaaaggagtatcggtgactacgggaaaaatccagaaacagatcacgaatcagaactctgaggttcacatgcctgaagatctgaagaagaaagggggagatctgctcaacctagggagtccaacaagggttttaaggaagatcagccattcacaaagggagaacggagcttacagatcaactgaagcacatcaaggagctaaaattgaagtttttcagaaacccatccagatggactggccactggtcactcagcccttaaacaaaagtttggtccaaggcaacaaatggaagaaagcagatgcaacccaagagaagcgtcggtcattccttcatgagttttgcaagaaatatggtagagtaaatgatcccaagttcaacctttttcatatagtatctaggatatatgtagaagacaaacacaaaatcctgtactgtgaagtaccaaaagctggctgctctaattggaaaagaattctgatggtcctaaatggattggcttcctctgcatacaatatctcccatgatactgtgcactatgggaagcatctgaaaacactggatagttttgacttaaaaggagtacacatgcgtttgaatacatataccaaagctgtgtttgttagagatcccatggaaagattagtctccgcatttagggataaatttgagcatcccaatagttactaccatccggtgtttggaaaggcaattatcaagaaatatcgaccaaatgcctctgcagaagcattaaataatggatctggagtcaaattcaaagaattcgcctactatttgctggatgctcaccgtccagtaggaatggatattcactgggaaagagtcagcaaactgtgttatccgtgtttgatcaactatgactttgtagggaagtttgagaccttaggagaggatgccaattactttctacagttgattggtgctccaaaagagttgacatttccaaactttaaggataggcactcctctgatgaaagaaccaatgcccacgtggtaaggcagtatttaaaggacctgagcacagccgaaagacagctcatctatgacttctatcacttggactatttgatgtttaattacacaactccacatttgtaatttgcattcatttttctaaagccctacatagatttaatgatgatggcctcaaataagctactgtaattgtcctacaattctctgtatg

SEQ ID NO: 30 - protein, 413 aa, Mus musculus
MKAKQVFFSVLLFGTAGLLLFMYLQAWIEEHHTGKIEKKRDQKGVSVTTGKIQKQITNQNSEVHMPEDLKKKGGDLLNLGSPTRVLRKISHSQRENGAYRSTEAHQGAKIEVFQKPIQMDWPLVTQPLNKSLVQGNKWKKADATQEKRRSFLHEFCKKYGRVNDPKFNLFHIVSRIYVEDKHKILYCEVPKAGCSNWKRILMVLNGLASSAYNISHDTVHYGKHLKTLDSFDLKGVHMRLNTYTKAVFVRDPMERLVSAFRDKFEHPNSYYHPVFGKAIIKKYRPNASAEALNNGSGVKFKEFAYYLLDAHRPVGMDIHWERVSKLCYPCLINYDFVGKFETLGEDANYFLQLIGAPKELTFPNFKDRHSSDERTNAHVVRQYLKDLSTAERQLIYDFYHLDYLMFNYTTPHL

SEQ ID NO: 31 - DNA, 4264 bp, Mus musculus
  CDS (561)..(2246)
ggagtcaggttgacgcgccctgggtgcggggtgctaggcgcggggctgggcgcgcgctcccgcggagtcggagctgcagaggcttccgagatcgtcttgggcttttcaggtcacctagcattggtatgaatctgctcctcaagaagagaaaatattctgaaggcatgtccgtttacctgtcaacgaggaacccataatgtcttctcatctccagtgtggctccatgctctctcctgatgctactccaactttctttggtgccatgggaaaaacctgaagcacgagcagagtcacctccaaatctgtgctcacttcattttgttgcatggctggtgcggaacagactattggaatttgctcttgtgtgttgcccttcgtacctgaaccaaagactgtgcctcattctctagtgcattgctgcctgctgtcttcctcgttcatttctgttgcttgtgccttcctactgtcttctgtcatcacaggacacccttaaagtttctcactggactcctcgagggctggcccatggctctctaagccagctgtccaaggtctctaccatgaggcattgcattaattgctgcgtccagctgttccccgaagacacacacaaacagcaagttgcctgccaaggaggcccccatcacagtcatcaggcgtgccccacttgcaaaggagaaaacaaaattctgtttcgtgtggacagtaagcagatgaacttgcttgctgttctcgaagtgaggactgagggcaatgaaaactggggtgggtttttgcgcttcaggaaggggaagcgatgtagcctggtctttggattgataataatgaccttggtgatggcttcttacatcctttccggggctcatcaggaacttctgatttcctcccctttccattatgggggctttcccagcaaccccagcgtgatggatggtgagaaccccagtgatgtgaaagagcatcactaccagccttctgtaaataacatctcctatgtaaaggactatccgagcattaaactgattatcgacagcattgctgccaggattgagttcacaaccaggcagctcccggacttacaagatctcaagagacaagagttgcatatgttttctgtaatccccagcaaattcctcccgactagcaagagcccttgttggtatgaggagttctcgggcaggaacaccactgacccctacctgaccaattcctacgtgctctactccaagcggttccgctctacctttgacgctctgcgcaaggttttctggggccacctgtcccacgtgcagggcaagcacttccgcctgcgctgcctgccccacttctacatcattgggcagcccaagtgtggaaccactgacctgtacgaccgcctccggctgcatccagaagtgaaattctcagccatcaaggagccgcactggtggacccggaagcgctttggaattgtccgcctgcgggacggactacgagaccgctaccctgtggaagattacctggacctctttgacttggctgcacaccagatccatcaaggactgcaggctgcctctgcagagcagcccagcaagatgaataagatcattattggagaggccagcgcctctacaatgtgggataacaatgcctggaccttcttctatgacaacagcacagacggcgagccaccattcctgacccaagacttcatccatgcctttcaaccagaagccaagctcattgttatgctcagggatcctgtggagaggttgtactcagactatctctacttcgcaagttccaataaatctgcagatgacttccacgagaaagtgacggaggctctgcagctgtttgaaaattgcatgctggattattcactgcgcgcctgcgtctacaacaacaccctgaacaatgccatgcccgtgaggctccaggttggcctgtatgctgtgtacctcctggattggctgactgtcttcagtaaggagcagttcctcattctccgtctggaagaccacgcatccaatgtcaagtataccatgcacaaggtcttccagtttctaaacctggggcctctgagtgagaagcaggaagctctgatgaccaagagccctgcatccaacacacggcgtcctgaggatcgtagtctgggacccatgtggcccatcacccagaagattctgcgtgaattctatgggcctttcaacacaaggctggcacaggtcttggatgacgaagcatttgcctggaagacaacgtgagagctgagtcccttctgcagaagctgggcccactgactctgtcattgccatgactttaaaagtctccaggcggagaactgtcacccacagcgtggagaaagccgctggagccccacccaatcctcttctcatgtccaaggcacctggtgtgggaactattttaaagctttccttgccatgttcagcagctctgctcttgattgcaaggctcgtccacctgaatccttggaaacgacactgggggtaggcttctgtctgttcccagagtgccttggatctcaggagggcagtggatctctggcacccagtgggtaggaagaggaagctacgggaggccatagaccagccttgccttccttcctccaggagagtttgtctgggtgtagggttggacagctctgggatccgattaaggccatgttctggggctgcccctcacgtgttatatcccagcaggccttgcataaatcgacaaccccatgtggacagagaacagagctgcttggagaatggaggctctttcgtttttgttcgtattcccaagtcagcccttcggtgacatcagaaatgagccctgttccctttgctgtgctcagaagaaactcagaagtcagcttttggcagtaggctcgtctctctgacctattctgcccatacactttgtttatgcccagcgtagagaaggagcgttttaaaacaaactgcttctcgcatttgactggccctgcctctcttttctgctcccttggatgttttccttaaggacttcagtttatatttggaaagggaagttttgctgttaatgccagaacagaacacctcaaggcatcgaacagtggaaaaggaaggaagtgaagccctccccatttgtgggggtcactggagagtgtggggatgtttttggttctggctggttacaggctcccccctgtcttcctgttggaggaatctttatccacattctctgtcatgtgcagccaaagttctgtaactcactaagggtgagtctcaagacacccttagaatgtcctcagaaacacatctcacaaactaagctcgggccacatgtggtgtcactaggagatagctttgatttttatgaacctttccaggcctgctagcagcttggttcgcaaaggatatgctgaaagtcttgtcattttaaaaaaaaaaaaagaccccagggaataccagagagaacaacttaaacccacttggcacacaggacaaggtggggatgtcacaaagcttttcattggagcgttgccaagggatgctgcctgtgaagctgtgttctcaggtcaggatcctgttcaccagcgtcagaaagtgtacacaccacatgcccgactgtcacttggacgtgtgagaaaagctgggctttggcagcaggaggctgtgcagttgctcgactgtgtgagttctgctgcggtccacccttgttggagatgggactctcagctcattggaaggttccgggagagctctgggcatcagcatggactgggctcgttctgctactgaaggggctgtgtgtcatgtggcctgtgttccaggacaattctcccttctctcacggtggccacttggctcggcagccagtgggaaaatctagcctgagtacaaggatgctttgggaagaaatgacgagagagagagagagagagagagagagagagagagagagagagagagcgagagagagcgcaagctcaagcgcaagcgtgtgagaaagagcacgagagagtatttatttggaataccgtgtgtgtgtgtgtgtgtgtgtgtgtgtgtgtgtgtgtgtgtgtgtgtgcgtgcgcgcgcgagtgtgcgcgcgaacactcgtgcttatactgagcagggccctctcagctgtcttgttgatggggcttctcctccattagagacatgatgcctttatgaattaatttctccaacccttcgcctgtcatttccaggacatatcttactgccccacaccaaccacgactctaagagattttttctgacgcactccc

SEQ ID NO: 32 - protein, 561 aa, Mus musculus
MRHCINCCVQLFPEDTHKQQVACQGGPHHSHQACPTCKGENKILFRVDSKQMNLLAVLEVRTEGNENWGGFLRFRKGKRCSLVFGLIIMTLVMASYILSGAHQELLISSPFHYGGFPSNPSVMDGENPSDVKEHHYQPSVNNISYVKDYPSIKLIIDSIAARIEFTTRQLPDLQDLKRQELHMFSVIPSKFLPTSKSPCWYEEFSGRNTTDPYLTNSYVLYSKRFRSTFDALRKVFWGHLSHVQGKHFRLRCLPHFYIIGQPKCGTTDLYDRLRLHPEVKFSAIKEPHWWTRKRFGIVRLRDGLRDRYPVEDYLDLFDLAAHQIHQGLQAASAEQPSKMNKIIIGEASASTMWDNNAWTFFYDNSTDGEPPFLTQDFIHAFQPEAKLIVMLRDPVERLYSDYLYFASSNKSADDFHEKVTEALQLFENCMLDYSLRACVYNNTLNNAMPVRLQVGLYAVYLLDWLTVFSKEQFLILRLEDHASNVKYTMHKVFQFLNLGPLSEKQEALMTKSPASNTRRPEDRSLGPMWPITQKILREFYGPFNTRLAQVLDDEAFAWKTT

SEQ ID NO: 33 - DNA, 1718 bp, Mus musculus
  CDS (374)..(1564)
acaaagcccggggggtccacgctggcgctggaggcgagccgggagcgagcccggggcgcggcgggacttggggcgcagggctgctacgggacgcggggctgctagcaggacgcgcgaccaggcgtgggttgcgatcgccacccccacggcggggcccgggacattttgggccctttggggccgcagcgagcggtggggaccaagacgagcagagtaccctgagcccggcaacttcggcccctccccgcccccacccggctgccctcagcgcgtccctctccatgtgcagacagccggctaggcgctccggctggcggcggatgggtgacctcttcctggccggggcgggctgttcgaggcggcggagccggcgatgaagaagaagcagcagcagcatcccggcggcggcacggatccctggccccatggggcccctgtggggggcgcccctccgtgcctgggcagctgcaagcgccggatcccgctgctgcctttcctgcgcttctccctccgggactacggtttctgcatggctaccctggtggtcttctgcctgggctccctcttctaccagctcagcgggggaccccctcgcttcctgctcgacctgcggcagtatttgggaaattccacttacttggatgaccatggaccacctcctagtaaggtccttcccttccccagccaggtggtgtacaacagagttggcaagtgtggcagtcgtaccgtggtcttgcttctgagaatcttgtcagagaagcacggcttcaatttggtcacatcagacattcacaacaagacccggcttactaaaaatgaacaaatggaactgattaagaatataagtaccgccgaacagccctatttattcacgagacatgtccacttcctcaacttctccaggtttggaggagaccagcctgtctacatcaacatcatcagagaccccgtcagtagatttctgtctaactatttcttccgtcgctttggagattggagaggggaacagaatcacatgatccgcacccccagcatgaggcaggaggagcgctacctggacatcaatgagtgcattctggaaaactaccctgagtgttccaaccccaggctgttttacatcatcccatatttctgtggacagcaccccagatgtagggagcccggcgagtgggcgctggagcgggccaagctgaacgtgaacgaaaacttcctgctcgtggggatcctggaggagctggaagatgtgctgctcttactggaaagatttttacctcattacttcaagggtgtgctgagcatctacaaggacccagagcacaggaaagcttggcaacatgactgtgaccgtgaggaagactgtcccctcccccgaggcggtgcagattctctaccagcgaatgagatacgagtacgagttctaccactacgtcagagaacaattccacctgctcaagcgcaagcttggactgaagtctcgcgtgagcggacctcccgtgagaccacagttcttcatccccacgcccctggagacggaggagccaatcgacgacgaggagcaagatgacgaaaaatggctagaagatatttataagaggtgatacctgcgccagattgtggcttctgtggcctcatccccccttttacaaagttctttggggaagagaaattctgaagggactttcattaatgctcgagtgcattaaaaagaacaaaacatt

SEQ ID NO: 34 - protein, 396 aa, Mus musculus
MKKKQQQHPGGGTDPWPHGAPVGGAPPCLGSCKRRIPLLPFLRFSLRDYGFCMATLVVFCLGSLFYQLSGGPPRFLLDLRQYLGNSTYLDDHGPPPSKVLPFPSQVVYNRVGKCGSRTVVLLLRILSEKHGFNLVTSDIHNKTRLTKNEQMELIKNISTAEQPYLFTRHVHFLNFSRFGGDQPVYINIIRDPVSRFLSNYFFRRFGDWRGEQNHMIRTPSMRQEERYLDINECILENYPECSNPRLFYIIPYFCGQHPRCREPGEWALERAKLNVNENFLLVGILEELEDVLLLLERFLPHYFKGVLSIYKDPEHRKAWQHDCDREEDCPLPRGGADSLPANEIRVRVLPLRQRTIPPAQAQAWTEVSRERTSRETTVLHPHAPGDGGANRRRGAR

SEQ ID NO: 35 - DNA, 5091 bp, Mus musculus
  CDS (318)..(1595)
ggcggcacccaaacagctaccccgtgcggagacccaaactttctactgccacttggagtctcgcggccgccgcctccgccccgcgcgtccggggcctgcccgtcagtccgtcggtccgcgtggagcagctcgggcgccgccgtgctcccgatccccgcagctgcagcgccgcagtcctggcccggacgcccgggcaagttctccagagttaaaagcgaagttcgggcgaggcgcgggccgagctgcctctgagcgcccccggcgtccccagtgcgcccagccccgccgggggcgccggtgacccttcggtgccagccatgtcgtccatcctgcccttcacccccccgatcgtgaagcgcctgctgggttggaagaagggcgagcagaacgggcaggaggagaagtggtgcgagaaggcggtcaagagcttggtgaagaagctcaagaagacggggcagttggacgagctggagaaggccatcaccacgcagaacgtgaacaccaagtgcattaccatccccaggtcactggatggtcggctgcaggtgtcccatcggaaggggctccctcacgttatctactgccgcctgtggcgatggcccgacctgcacagccaccatgaattacgggccatggagctctgtgagtttgccttcaacatgaagaaggatgaagtgtgtgtaaatccttaccactatcagagagtagagacgccagttctacctccagtgttggtgccacgccacaccgagatcccggccgagttccccccactggatgactacagccattccattcccgagaacactaacttccctgctggcattgagccccagagcaatattccagaaaccccacctcctggctacctgagtgaagatggagaaaccagtgaccaccagatgaaccacagcatggacgcaggttctccaaacctctccccgaatccgatgtccccagcacacaataacttggacctacagccagtcacctactgtgagccggccttctggtgctccatctcctactacgagctgaaccagcgagttggggagacattccacgcctcacagccatccatgacagtagatggcttcactgacccctccaactcggagcgcttctgcctgggcctactgtccaatgtcaaccggaatgcagccgtggaacttacaaggcgacacattgggagaggtgtgcggctctactacatcggaggggaggtctttgcggagtgcctcagtgacagtgctattttcgtccagtctcccaactgcaaccagcgctatggctggcacccggccactgtctgcaagatcccaccaggctgcaacctgaagatcttcaacaaccaggaatttgctgccctcctagctcagtctgtcaaccagggctttgaggctgtctaccagctgacgcgcatgtgcaccatccgtatgagcttcgtcaaaggctggggagcagagtacaggagacagacagtgaccagcaccccctgctggattgagctacacctgaatggacccttgcagtggcttgtcaaggtcctcacccagatgggttccccgagcatccgctgttccagtgtgtcttagagacactaggagtaaagggagcgggttggggagggcgggcttggggaaaatgaccttggaagagaactccatccaacttggtcttgtcaaagaacaccgattccactcaactaaggcaccagcctgtttctgagaccacagaagaaaaccccagggatggatttatgaacagctgtgtctgctacatacacgtgcccctgtctgaaggccaagtgatggcttctgttctggtggcttgaactaacaggtggtgtatcgccacctgactccttgtttaatgacagaggtctgggatgtcacagtccaaaaggaaagtgcctttctccatggctggagtatggagtttacctttggagaagttgtaatggagcatgccctgtcccaccactctcagagagggtgtacctgtcaaactggatggcctacataggtactcccccctacccctaggatgcagagagacgggaacacgccggagggtagagctggggagaacccattcttccttggaaggatccgctgaaggtcagcgtataggtgatgtacagttcctaatatcacatcagtctcagagtgttcacaggaagcagcaagggcactcgtggagtatgtgtcctgggtgaggtggcaccacaccgaatgaatgcatctctgggagctggcaccacaaccctgatgtataggctgtgttggtttatggagacaagttgcatcaatgaattcacctagcataggctctttgaaatgtcctctgtttgataaaaaacaatcctgggtacgtatgttggctggaaaaccacaatggaccctgccactgcttcttgccctgaggtttggaagctgagagttatagaagccaattcaccaggaggtaagacatcccaggctgacatgggcaaatgaaaagggctattaaaatttttttacaccttggaaaattgcaggcttggtgcagagcgctctgtcatcttcaccctgggatgtaggattacctagctatggtaaaggattgccacagcaaactgtgacactgtgtaatgagcactgttcccagcggcaattacagagaaaacgagtgtaaattatttcaactggaaaaaagtccctttcttggctgttttagaacagggtacacaggatcgccacctgcaactggtactcgcttcttggctgctgcttctgttgtgaaaagacgagcccatgtttctgcatggatttcccatggaagtcctgtcctgctacagaggggaagaaagtgtaccctccaatgtgataaatcttctgatgcccccagactcttggagcacatcctggtgcccctcctgcaggagcctgtggcatatttccagctgggcatgctgatcctccttgagacacagatgcctgtgtgagtctccgttgatacaattctgaacccctcaggttctctgaaagggcacagaccatgggcgtgaacattgtgccgtacctgagttggtctgtggactgctgcttcagacacacgagtcctcggaactgcctggctgcctgtcacgcatgctctgagtcagaacacaccaacgctctgctgtggctcctagggaagcattcatggtcctctgttatcagcaggggtttatgtcacttgctgtccggtttcctaggggcttcctgtgccccttccccagctatcctccaggtggctagggacagtctattctgctgcaactggaaagtagagggaaccggcactgctcagagcagatggcggcttctggaggcacacagtgggagtacaccccttcatgttattggccagttgctggagaatgctgtaggagaaaattctaggcaggtctactcttggcatccctgagagtcaaaggcttggagtctaggaaaggtcacaccatgatggagaacacaggtcatttgggtacgtgtaatcaaagtgccctcccaaatcagttctccttttcgtatgaacagcatctctacttttaaagaggagttgaggatcgagaagatgacagtgcagcagtgggtgtggcctgactacatgtgctgttccagccctgggtgcccactgacaccgacccccaggcagaggcctttgtcttcagcactcctgagaagttggctctttaccccttctcctctgctgcccctccttcctgctggttcaggtagccccagccacgtgggttagagtcctgtgctggcctgccatggcagctggctaccttccagaccaactgtagagatacctggcattttgaagaaagcctagactggagagcagggcctctcttgggaaggacacaaggcgggcaaggctgctggcagacttctccactgacctgagtgtgctttttttttcccctaaatgtattgcatcaagcctcagtgcttatggagtgcagtggtcttcatcttccccaacttgcttctcagagctgggatgtattccagagcctgatgtttttattcaaaccacagaaaagttttctttaagtagcctgtgcagtcatgcatgtgcctgagttgtctggagcagaggcaaacatctgacttcattcttagccccaagctgccatttctgagtccttgagaggctgagaaggctctagctttgtactgtattcttactgtgtgactagatgcgtgagcgctttacattagaaggaacctggttagagctcgctcctcctgtctttgtgtggcatttgtgttccattaccggcccctttaagtaacggccttcacagcaccttcccagtgggtagaaagccacacaccaggatgtgggtcaaccatgaagatgtggcattgcagacgggggaacatgtggatgcatggctatcgccctgaacagtccctgcagctacttgtgttaacacagaactgatgtttagcattctgccgctttcgtatttatgtaacaattccttaaagccattcaaatggctaactatttaatttctttaggacagttgtaaaggtctctctcctgaggacaatgacttggaagaactggggcacagccagtcccagacactggtggaggctgcagtgactttttttggctcaagatccacaagcattagagtagactgggccaacaagtcaacaagtggtggcgtgtgcaaacgggctgccctagtcaagcccagtcccttcaacagtatgtctgatgcaccacaggccctccctactggaagtgggaacttcaaatggaaattggagccatcttttatcccagaagcctttgctgctgccagggcaagtgggctggtgtggactcttgtttaggaggctgaggttcttgtcactccttagccagccaggcctttagtgtctttgtaaaaagcatgtatttatagtgttttagatttttctaacttttgtatcttacagcattgtaccccattgttaaagagccgtgtcccctcttcttataaacgcccttttaataaacttttcaccgtaaagctcctgagacaggagcacagtctgt

SEQ ID NO: 36 - protein, 425 aa, Mus musculus
MSSILPFTPPIVKRLLGWKKGEQNGQEEKWCEKAVKSLVKKLKKTGQLDELEKAITTQNVNTKCITIPRSLDGRLQVSHRKGLPHVIYCRLWRWPDLHSHHELRAMELCEFAFNMKKDEVCVNPYHYQRVETPVLPPVLVPRHTEIPAEFPPLDDYSHSIPENTNFPAGIEPQSNIPETPPPGYLSEDGETSDHQMNHSMDAGSPNLSPNPMSPAHNNLDLQPVTYCEPAFWCSISYYELNQRVGETFHASQPSMTVDGFTDPSNSERFCLGLLSNVNRNAAVELTRRHIGRGVRLYYIGGEVFAECLSDSAIFVQSPNCNQRYGWHPATVCKIPPGCNLKIFNNQEFAALLAQSVNQGFEAVYQLTRMCTIRMSFVKGWGAEYRRQTVTSTPCWIELHLNGPLQWLVKVLTQMGSPSIRCSSVS

SEQ ID NO: 37 - DNA, 1983 bp, Mus musculus
  CDS (80)..(1171)
tggagctggtcggttatggttggaactttccctgcccttcgttgactgagtgaaccagccagaaatacattcccaggaaatggcctctaaatcttggctgaattttttagtcttcctctgtggatcagcaatagggttttttttatgttctcaactcttgagtattttgttgcgagaagaggctgccattcagcctaacatgcttcacaatgaccctcatgcaaggcattcagatgacaatggacacagtcacctcaaaggacagatgaacttcaatgcagattccagccaacataaagatgagaacatagacgttgctgagaacctctatcagaaagttaaaattctttgttgggttatgacaagtcctcaaaatctagagaaaaaggccaaacatgtcaaagctacgtgggcccagcgttgtaataaagtgttatttatgagttcggaagaaaatcaggacttccctactgtgggattgaaaaccaaagaaggcagagagcaactatattggaaaacaattaaagctttccagtatgtacatgaccattatttagaagatgctgactggtttatgaaagcagatgacgacacatacgtcattgtggacaacctgagatggcttctatcaaagtatgaccctgaacaacccatttactttgggcgaagatttaagccctatgtgaagcagggatacatgagcggaggagcgggctatgtcctaagcaaggaagccttgagaagatttgttaatgcatttaaaacagaaaagtgtacacatagttcctccatcgaagacttagctctgggaaggtgcatggaaattataaatgtagaagctggagattccagagataccattgggaaagaaaccttccatccatttgtaccagaacaccacttaatcaaaggttatctaccaaaaacattttggtactggaattacaactattatcctcccatagagggtcctggatgctgttctgatattgcagtttcttttcactatgttgatgggacaactatgtatgaattagaatacctcgtttatcgtcttcgtccatatggttgtttatatagatatcaacctgccttacctgagaatatactgaaagaaattaatcaagtaaacagaaaggaagatacaaaaataaaattaggcaacccctgaaagcagaacataagtggtctacattgcactgaaggactcttgcctttctacggaaccctgaaatcccagtgaggaactcacctgaagtgaacattccatatagaaatctttcaaatggatgactataaactgaagcatttaaagagctgtgaagtttgctaaaacgtgtcttgatacagtaatatataaatataaataaatatatatgaagaatttgtgtttttaaatggtgacagaagagctagaaagactctgtttctgactgttcccaacagtgtatgttgttatcactacagttggagtggttagtttataatcaccttctgcaccacaccagttagaacacaatgctgaacagatctgccttaaagaaagaaagaaagaactgttgctcagtgttgtttctcagctgatatgtgtatgctgcaaaccagccttccagttgccgaatgaaaactttcctgtgattatattatttataattggcagtgcttcctcaagaagaataagaagggcacttaccccaaagtgcagtaattaaccacatttccaattattccgagccttgactccattagatacttagttctaaagataatcaacccgataccaagtccccattgtcccctttccaaatgtttgaccctgaataccatttttcactattaatgctaatttactcaaagcattggcagcattctgtaaatcatgcattttcaaatatgttgttccccgattcacccattgttaattgtaggtaacaattaaatttaatgctgtttggacaagcaaaaaaaaaaaaaaaaaaaaaa

SEQ ID NO: 38 - protein, 363 aa, Mus musculus
MASKSWLNFLVFLCGSAIGFFLCSQLLSILLREEAAIQPNMLHNDPHARHSDDNGHSHLKGQMNFNADSSQHKDENIDVAENLYQKVKILCWVMTSPQNLEKKAKHVKATWAQRCNKVLFMSSEENQDFPTVGLKTKEGREQLYWKTIKAFQYVHDHYLEDADWFMKADDDTYVIVDNLRWLLSKYDPEQPIYFGRRFKPYVKQGYMSGGAGYVLSKEALRRFVNAFKTEKCTHSSSIEDLALGRCMEIINVEAGDSRDTIGKETFHPFVPEHHLIKGYLPKTFWYWNYNYYPPIEGPGCCSDIAVSFHYVDGTTMYELEYLVYRLRPYGCLYRYQPALPENILKEINQVNRKEDTKIKLGNP

SEQ ID NO: 39 - DNA, 4263 bp, Mus musculus
  CDS (602)..(1576)
ccgagaaggagagagcggcacaggtttctgccgcggcgctcgaacgggagcttagggacgcagcatcctcggtgtccacccgggatcgcagctgcaccggccgacatgtgacggaggaaagccctcagcattcccggcaaccgccttcaaaggggtcccggaacgctcgcccaggccagcagctggatcctcttagccctcagcgacgcgggacaggagcggcaaggtacaacgcctggtacgttccaggctcgcactcgggaaaagagcgcagcggggaccgccgcctggcttgcactcggcacaactccgggacctgcggagtgcggctggaccgtgtccagattcccgccgacttcatccagagtgacccaagaccctagccggtcctcctgcaccccctggagttctagcttgccctgcatctgcgccgaccccacaacgcccaggtccccgcaacgccccagtccggaaggggagcgcgcttgcggcttctccctgattgcagacgctgccgctgtcctaactgcacacagaggtcccctacccggagcctagtccttgcagggggtggtgtccgagacgctggggagcgcgcaccatgaagtccgcgctgtgcagccgcttcttcatcctcctgccctggatcctgattgtcatcatcatgttggacgtggacccccgcaggcccgcgccccaactcacttcccgtccctacttctctccccatgcagtgggctgcgggggctcccgagtcccgctccgacggagtagccccgggcgtgacgctgccgagaagcggaacgagtctcggcctcagctgcagccggagccgcgcttgcccaccatctatgccatcacacccacctacagtcgcccggtgcagaaagccgagctcactcgcctggccaacaccttccgccaggtggcgcagttgcactggatcctggtggaggaccgggcgacgcgcagcgagctggtgagcagcttcctagctcgggccggcctgcccaacacgcacctgcatgtgcccacgccgcggcgctacaagcgaccgtggctgccgcgcgccaccgaacagcgcaacgcgggcctcgcctggctgcgccagaggcatcagcatcagagcgcgcaacccggcgtgctcttcttcgccgacgatgacaacacgtacagtctggagctcttccaggagatgaggaccactcgcaaggtttctgtctggcctgtgggactagttggtgggcgacgctatgaacgtccgttggtaaaaaatggcaaagttgttggctggtacaccggatggagagaagacaggccttttgccattgacatggctgggtttgctgtgagtctgcaagtcatcttgtccaatccgaaagctgtatttaagcgccgtggatcccagccagggatgcaagaatctgactttctaaagcagataacaacagttgaagaactggaaccaaaagctagtaactgcaccaaggttctcgtgtggcacactcggacagagaaggtcaatctagccaatgagccaaagtatcacctggacacagtgaacattgaggtgtagccgccaggaccagcaggtgaggacaggaagtctggagcgtgggctgctgcccattcaggtactgttcctttgccttcagcctttccttaccgccgatggacggccgtttcgacagctacctggatgctacaaaatgtgttgtcttcatttgtctgcatgtgttttctgaactggaagagttttgtagagtgaagccacatgtcactgaaaaggacaacgaagtatctcacactgctccctccctccctccctccctccctccatccctccctcccacaagccatctcaatactgctagaatgacaggttttagaagctgatgaggagtactctgtcaattaaatattcccagttaaattcttggcttcacaacatgaactatttggaaagctgatggcagtgtacactgtaggaaataaaacccacaaataaaaaggtgtgtggattcatctgtttaatatagggaaataacattttgtcaatactaggcaccataaaatgtaaacacaattactgtcataaacctggatataccttcaaggattaaaagtggctttgttttagttaccctgtgtgcatatagtgcagaaaaaggtcttcatattagcactatgtacatgagaagaggcccacattacaagaggcaatggaaatttgaattctgaaaccttcattgagttttacagtagcatccaggtttatctccgttcactaaccacgttccctgttaagcacatcataacaacagcacagtggagtgagtgatgaataaaagaatttgatacactagaaaacagtgctcagtgatacatttacattctatttatatgattaaacatttgatcatacagtacctttctacaggattactggctaatttgggggtggggtttatactgttagaggtattactaacatgataactacttcccttatatgcaaacgttagagctataattttgcaaataaaactgattatgcaagttaactgagcagcttcccaatgtggcttaggacatcatggggagtatgagcaaagctgccctccacacagatggtttctcaacctgctttcacatccaatttaatcagcacagaccaacacggtcaatcaggtcattcttaatgtgagcaaatgggggaaagaaaaaatacgttatgtacatgaataaatgggaactagatgcctttgcagcgaggaatgttggggtcggttctggatggaactctgtgacagcttcttacttccacagctgtgtgctcagcgtctgccccgatgagcttccagaccaccctgctgtcgtgctgggaggctggccaaaacctgcactagcgttagcactactgaggttcatggcagccatctgctgattcatctgcgaaacacaaaaggcttggtttacaaggaggaccctcttttcactcctgactccttagctacagccagacacttcatcagatgtgaaaattagtccagacactggatgtggactacactgaagtctttctactgaaatcagttacttttttcttacttttttctgagttaggtgttacttataaagtaatccataattagttcactttctagattaaccacaactgaaaacacatcaaagtaatctttgagtttatatctgagaccatcactcttctgtgactccaagactgagacattttagaaagtaaaacatctaaaattaccctaacatttacaaaatactcatacctctctattaacatcctaaatgaccttaatatacttcctaaacattaaaaaggtcatatagcatatgttttaggtttatgttataataacataaacagctaaaattaaatttcattatatgctatacaataaacaactacatgtacttttctttcaggctaaaatccagtatatttatcaggctcttagcacagtagttctcccactacccagtttggtgacggaacaagagatgcaggatttacctaaaatagtttcaaacctatgttcaacattatctgtgttctttgtgcaaattttttttaaaacccaagacataataagcacacatgtgtctacagacaggtagcttccatcttcccttctgctcactcttgtggccttgctgttcttcactggctctcctgtactctaacctgagtcatgtgagcctgagcatcctggtcacagaaaagctgttcctagtatctgacatcattcccgaggtttccaatgcataagcagtgtgtacgtatatacctataaaccgcagctgtcacagcacacgagcatttgtaagatacacggtgattctgtgaatgttagttagcagacattgttctgaaggtaaactaggggtcttagtggtcctggttcctgtgagatgctcggtggaccatcatgcaggaatgggaatgggaccatgagcactaagtcaaagcctctgatgtggggtaacagcagaaatgttactctaacctaagccagacagacatggaatttatcagtgaatacctgagggaagcagggtttacattctcaccatggggattcctgagccaatggatgttcctcatacatcaacaggcagtaaccacatgacagcggcagccttgatccctcataaaactgtgctttgaaaatatgaagctggatttctatctcactgataagaattaataaaaaaaaaaaaaaaaaa

SEQ ID NO: 40 - protein, 324 aa, Mus musculus
MKSALCSRFFILLPWILIVIIMLDVDPRRPAPQLTSRPYFSPHAVGCGGSRVPLRRSSPGRDAAEKRNESRPQLQPEPRLPTIYAITPTYSRPVQKAELTRLANTFRQVAQLHWILVEDRATRSELVSSFLARAGLPNTHLHVPTPRRYKRPWLPRATEQRNAGLAWLRQRHQHQSAQPGVLFFADDDNTYSLELFQEMRTTRKVSVWPVGLVGGRRYERPLVKNGKVVGWYTGWREDRPFAIDMAGFAVSLQVILSNPKAVFKRRGSQPGMQESDFLKQITTVEELEPKASNCTKVLVWHTRTEKVNLANEPKYHLDTVNIEV

SEQ ID NO: 41 - DNA, 1599 bp, Mus musculus
  CDS (175)..(1182)
gaagtggtcccaggtgtcgctcagagtcccgcgttcccgggaactagtaagggagcctggggaaggcggggcgcggcccgggcggccggggcggggccggcgggagggcggagggccgggcggacctgtccctgccggtcccggtggaccccgcctgccccggtggcaccggccatgaagctgaagctgaagaacgtgtttcttgcctacttcctggtgtcgatcgccggcctcctctacgctctggtgcagctcggccagccttgcgactgcctccctccgcttcgagctgcagctgagcagcttcggcagaaggacctgaggatatcccagttgcaagctgatctccgtcgcccaccccctgtcccagcccagccccctgaacctgaggccctgcctactatctatgtcattacccccacctacgccaggctggtacaaaaggcagagctggttcggctgtcccagaccctgagcttggtgccccgtctacactggctgctagtggaggacgctgagagccctaccccgctggtctcggggctgttggccgcctctggtctcctctttacacacctggctgtccttacccccaaggctcaacggcttagggaaggtgagccaggctgggtccggccccgaggagtggaacagcgcaataaggccctcgactggctccgaggaaaagggggtgctgttgggggggagaaggatccaccgccaccagggacccaaggagtcgtgtattttgctgacgatgacaacacctacagccgggagctctttaaggagatgcgttggactcgcggtgtctcagtgtggcctgtggggctggtgggtggcctgcgatttgaaggtcctcaggtacaggatggccgcgttgtgggtttccacacagcatgggaacccaacaggccctttcccttggacatggcgggatttgcggttgccctgcccttgctattggctaagcccaatgcccagtttgatgctactgcaccccggggccacctggaaagtagtctcctgagccaccttgtagatcccaaggacctggagccacgggctgccaattgtactcaggtactggtatggcacacccggacagagaaacctaagatgaagcaggaggagcagctacaacggcagggccagggctcagacccagccattgaggtgtgatggcaacctcaccctgacttctaccttattttaggcacataccttgtgggactgggctccaggccagcccaggatgtggttttttttttaaagacctgacccctgagaaccagaggcagcccctccaaccacagggtgtgtcccagttgcttccctcccttcccccagcctgccatgtggcactgcccacatgttggggacaagcagcttctctagtgagccagatcagccccatctggggcggagcaggacagatggactcagaaaggagggtagctgtgggaaaagggtaacttattggggacaagcatgcagtggggggctagaggagctgggctggaccctccccacctgagcatgcgatccccttcctacctctagaataaagaatctcaacccggaaaaaaaaaaaaaaaa

SEQ ID NO: 42 - protein, 335 aa, Mus musculus
MKLKLKNVFLAYFLVSIAGLLYALVQLGQPCDCLPPLRAAAEQLRQKDLRISQLQADLRRPPPVPAQPPEPEALPTIYVITPTYARLVQKAELVRLSQTLSLVPRLHWLLVEDAESPTPLVSGLLAASGLLFTHLAVLTPKAQRLREGEPGWVRPRGVEQRNKALDWLRGKGGAVGGEKDPPPPGTQGVVYFADDDNTYSRELFKEMRWTRGVSVWPVGLVGGLRFEGPQVQDGRVVGFHTAWEPNRPFPLDMAGFAVALPLLLAKPNAQFDATAPRGHLESSLLSHLVDPKDLEPRAANCTQVLVWHTRTEKPKMKQEEQLQRQGQGSDPAIEV

SEQ ID NO: 43 - DNA, 2974 bp, Mus musculus
  CDS (197)..(2203)
gataggggccccaggcttcccacagcatggagacacagggagtgtggagggtgccccccagcccacggacaacaccttcactccgaagtgtgagattgtgggcaaggacgcactgtcagcactggcccgggccagcaccaagcagtgtcaacaggagatcgctaatgtagtgtgcctgcaccaagctgggaacctaatgcccaagtctgtgccccggcactgccagctggctggcaagatgagccctggcgtccaatgggaagagatccgggcccagcagcctgttggtggccctccagtacgcatcgcctacatgctggtggttcacggccgtgctatccgccagctgaagcgtcttctgaaggccgtctaccatgagcaacacttcttttatatacatgtggacaagcgttccaactacctgtaccgggaggtagtggaactggcccagcactacgagaatgtgcgggtgacaccttggcgcatggtcaccatctggggcggggccagccttctgaggatgtatctgaggagcatgaaggaccttctggaaattcctggctggacctgggacttcttcatcaacctgagtgccactgactatccaacgaggacgaatgaggagctggtagcattcttatccaagaaccgggacaagaatttcctcaagtcacatgggcgagacaattccaggttcattaagaagcaaggcctggaccggcttttccatgagtgtgattcccacatgtggcgcctgggtgaacggcagatcccggcaggcattgtggtggatggtggctctgactggtttgtgctgacacgcagctttgtggaatatgtggtgtatacagatgaccccctcgtggcccagcttcgccagttctatacatacacattgcttccggctgagtcattcttccacacagtgctggagaacagcccggcctgtgcgagcctggtggacaacaacctgcgggtcaccaactggaaccgcaagctgggctgcaaatgccagtacaagcacatcgtggactggtgcggctgctcccccaacgacttcaagccacaggacttcctgaggcttcagcaagtctccagacccaccttctttgcccggaagttcgagtcgactgtgaaccaggaagtcctggaaattttggacttccatctatatggcagctacccacccagcacaccagccctcaaagcctactgggagaacatctacgacgtggccgatggccctggcggactcagcgacgtcctactcacagcctacacagcctttgcccgtctcagcctgcgtcatgctgccactgctgtatccccactggccactgcagtctgcaggtttgagcccagggggttgccgtccagcgtgcacctgtatttctatgacgaccatttccagggctacctggtgacgcaggcggtgcagccctcagcccaggggccagcagagacacttgagatgtggctgatgccccagaggtcgctgaagctgttggggcacagtgaccaggccagccggctccagagtctggaggttggtactgagtgggaccccaaagaacgtctcttccggaactttgggggcctgctgggaccactggatgagcctgtagccatgcagcgctgggcccggggccccaacctcacagccactgtggtctggattgaccccacctatgttgtggccacatcgtatgacatcacggtagatgcggataccgaagtcacgcagtacaagcctccactgagcctgccactgcggccgggagcctggactgtccgcttgcttcagttctgggaacccctgggtgagacccgcttcctcgtgctgccgttgacgttcaaccgcaaactacctctcaggaaagatgatgccagctggctgcatgcgggaccaccccacaacgaatacatggagcagagtttccagggcctaagtggcatcctgaatctgcctcaggcagaggccctggaggaggctgcccggaggcacacagagctcacgggttctgcacttgaggcctggacagatggggaactgagcaatttctggtctgtggcaggattgtgtgccataggaccttctgcttgtccctccctggagctctgcagactgaccagctggagctctctgtctcctgaccccaagtcagagctggggcctgtcaaagctgacgggcgactcaggtagcaggaccccagccagcacaacccggaggagccggggaattgcaccttacagacaacggagggacatctctcccacagacaagaaccagaggcccaggcagtttgcccatttgagccaacaggaacctacactgagggcagggaaggtgggtgcagtactccctactgctaacggccctgcaagagaccagcggtccactggagccatgaggaggatcttcctctgctcctcgttagtttatacttttttaataattaaaatgagtggcagagggaggacgatgtgcaatagggctcagagcagccccaggatatgggccatggccatggggagcaccacagagcatatcggtttctgctctggggaggcatgggctgcctccctctccagagcctccatccccagcctgcatctgaactgcgcactgctgaggctctgaggcccggaggacaggtgatctgtgggggctactgtggagttggactttagtgagcaggcctctcctctgggagccagggagccctagagcagagctggggccggctctcgggcttccctaggaaatgcgcgccatcagggcctgctgagatccatgaagtttccctactggaggaggaagcccactggcggcacaggaaggctgtctgtcagagctctcacaagagtcgagttgtgcctgaagctcagtgcgaagtctgaaatatgcaacagaggaaatatatctctatctctccaaaaaaaaaaaaaaaaaaaaaaaaaaaaaaaaaaaaa

SEQ ID NO: 44 - protein, 668 aa, Mus musculus
MPKSVPRHCQLAGKMSPGVQWEEIRAQQPVGGPPVRIAYMLVVHGRAIRQLKRLLKAVYHEQHFFYIHVDKRSNYLYREVVELAQHYENVRVTPWRMVTIWGGASLLRMYLRSMKDLLEIPGWTWDFFINLSATDYPTRTNEELVAFLSKNRDKNFLKSHGRDNSRFIKKQGLDRLFHECDSHMWRLGERQIPAGIVVDGGSDWFVLTRSFVEYVVYTDDPLVAQLRQFYTYTLLPAESFFHTVLENSPACASLVDNNLRVTNWNRKLGCKCQYKHIVDWCGCSPNDFKPQDFLRLQQVSRPTFFARKFESTVNQEVLEILDFHLYGSYPPSTPALKAYWENIYDVADGPGGLSDVLLTAYTAFARLSLRHAATAVSPLATAVCRFEPRGLPSSVHLYFYDDHFQGYLVTQAVQPSAQGPAETLEMWLMPQRSLKLLGHSDQASRLQSLEVGTEWDPKERLFRNFGGLLGPLDEPVAMQRWARGPNLTATVVWIDPTYVVATSYDITVDADTEVTQYKPPLSLPLRPGAWTVRLLQFWEPLGETRFLVLPLTFNRKLPLRKDDASWLHAGPPHNEYMEQSFQGLSGILNLPQAEALEEAARRHTELTGSALEAWTDGELSNFWSVAGLCAIGPSACPSLELCRLTSWSSLSPDPKSELGPVKADGRLR

SEQ ID NO: 45 - DNA, 5532 bp, Mus musculus
  CDS (369)..(1427)
ccagtgcacggcggcggcggcaccttccttctcgcatcccggtcgcccggcccgtgaggaggcagcggcggccgcaggcgccagcagaggaggcggcggagcatcgagcagaggcgaggcggaccggcaggagcgcgcggccggagccacgcatcctcacacttgcacaccaactcctgcgcctttcaccctctagaccagagtcggggcctcggagagcctcggtgaagctagcgggagccagctccggagcccagccagctctgttctgagcctccccggtgctggtacccccgcggcggggtccccgcttctgcgctcgggacgcgcctcccggacagccgggtccccgcggccaggacaaagccatgaagccggcgctgctggaagtgatgaggatgaacagaatttgccggatggtgctggccacttgcttcggatcctttatcttggtcatcttctatttccaaagtatgttgcacccagtcatgcggaggaaccccttcggtgtggacatctgctgccggaagggatcgagaagtcccctgcaggagctctacaatcccatccagctggagctatccaacactgccatcctgcaccagatgagacgggaccaggtgacagacacctgccgggccaacagtgccatgagccgcaagcgcagggtgctgacccccaacgacctgaagcacctggtggtggatgaggaccacgaactcatctactgctatgtgcccaaggtagcgtgcaccaactggaagaggctcatgatggtcctgagtggccggggcaagtacagcgatcccatggagatcccagccaacgaagcccacgtgtcggccaacctgaagacccttaaccagtacagcatcccagagatcaaccaccgcttgaaaagctacatgaagttcctgttcgtgcgggaacccttcgagaggctggtgtctgcctaccgcaacaagttcacgcagaagtacaacacctccttccacaagcgctacggcaccaagatcatccgacgccagcggaagaacgccacgcaggaggccctgcgcaagggggacgatgtcaagttcgaggagttcgtggcctacctcatcgacccccacacccagcgggaggagcccttcaacgagcactggcagacggtctactctctctgccacccgtgccacatccactacgacctcgtgggcaagtatgagacactggaggaggactccaattacgtactgcagctggccggagtgagcggctacctgaagttccccacctatgcaaagtccacccgaactaccgacgagatgaccacggagttcttccagaacatcagcgccgagcaccagacacagctgtacgaagtctacaaactggactttttaatgttcaactactcagtgccaaactacctgaagttggattagagggctgcggaggggagggggaggggtggttgggggagagggagagaatcctgctttttaatttaagatttttatttgtcaaaagaattctatggagactgggttattttgtaagttaatatgtcttcgggggagatgctgcgagcggcatggttaagaattatttaaaaattctccacggggaaggacagctgtctttgcaggggagcggggtggaatatccctgtttttagaagtggatactgcaacactgtctccaaggtgtccttgtgttctggtgaagtccacaaactgcattccataaagtctaatgaatcttatttatagttatttaaacgtggtctgtggcagcagcctgctcctgtctgtgcagaggagagtacagtctgtgcttcgctggctatggctcattgggggaggggagggggttatctcccccactgaccgtgtgtagaatcccatggtgacagctgcgggatgtgtgcctgtggccattcaagaaaccctgctagaaagaagtcaattgtccttgcactaagaaacagggatgagactggttcaaaccgattgctagcaaccccaaagtcccttgtagtaaagaaatgcaaatttttttaacccaagaagagagcagatgatagcgatttctctttaccgaaattagcaggctataagaataggctttctgtggaaatctttaagtggcactttccagtgctagcgaaggcagaataatggttgttttctagtcaataagtattgagcatctactattctccaggcttgactgcagatagcacattgagtgaaaccaggagccttggccttcagcaagctttatgtgaacacagggcatccagctcaacagtgaaccaacacccccatccctttggtcctcacacagactttagactattctcaagtggtccagagggtccaggaccctagcaaatttgcgtatttctacagttcaaatttgaaagccacacatttggatcaggccacttaactagctaagtagttcattgcccaacacaagaaatgacagaaacccttttgatcatgtatatctagtgcagtgtcttcatagtagaagcctataagccacaatattctccaagcgcaaggatggaggactgctgtgattgattagtgatgtctgccttgactaaggaaagggaggaacatttctcaagggcctcctactgtgatctccagtccacatcttgtcctgcctgccacttaaaacagtgcactgcatacatttggagactggcggagttcaagtgtgaaggtcaaaggtggagtgttggtacccacaggcacatgcccaccctccatgtcctgcattggcagagcatccaggacctccctctcctccagaaggttggaaatttgctctttcctgttccttgctgatcaatgcccaaacctattcctcatttctagttgtttctgatactttgcggttgacatttgagccacattcccatgacagccagtgtccgggtacgtagcagatgctgtacatagttctgatgataagggagctggtaggctgtaaattttgctttttgtgtttttgtggggttttgtttgttttgatgcagtatagagtgaagggggtaagaatattctgaaaaacataaaattgagtaatttattcacagaaactattaaaatggtattgttaggctcacaaggaggctgggggcagcggtaactgctacactgtctcgcagaggaaattattttatttaatgtgagagaaatgtgagccagagtggctgcctgaggctatttataaaacgcaggtaccttccccaatttctctcccctcgcctttaataaatagatttatgtcatgtgtgtgaccaagaacagtgacagatcttggctgtccctggaatagagaggcagtcatggtgctcaggccctgggcgtggtgtagctgatgcatgtcccaccgagcccaggctctccaaggcatcagtttgtggtcacatatccttagaaaacaaaattaagtcaggttcctgtctgcaggagttttcagagccttgaatacatagtctgccttgtgaaactgggagaatgtagcatttctgggctgcatgacctaggtatcctgtatggcagaccatgtcttagctggtttttcacacagtagccttcagtgagggtgatctagcctgtctgaggtctgcaaagattgacagctgcttcctagatccttccacaaatcagcagaaacagtgtccatctgggaactttatgagagtgttgctttacctagttggggtgtcttcatctggatactgccccagagagtgatcccgtccctccaagcacagcggagggaagcctagggtgttatggagtggggcaggtggcttaaggacaccttaaaggccacttggtggggataatggcctcgtggggatactcctcacttccacccacagtttggaactgttcactgtgtgcttcaggagttttaaatggagattcgtgttgatttccatggtccgcatctgtttgtcttccgtctgtggtccagtgctgtgtgcagatggtacttactccctttctctctcttacagggagaaagactgtccttctggggagagtgtatcaatacgcttccagaatgagggagtctttctcctgtcttgtgttcacacttcctagtgacaattttcaaaatatgtatcggcttgactgtttattgtagagtgtagaaaggtttctgtgttcctggctaaagacatccattggtgaaatgtgcttggaaatccaagtgttttctactgaggaaaaaaaaatgttggaaaagctttctcctgcatgcctcttgtgcttagctagaaaggggagctgccagcgtcccagctctgagccactgttcaaaggtgcagctgtgttttaggactaggtacaggcagagtgataaggacatgcctgcttagtgtactcatttatcgagaaagtttagactatgagccacaggggtccctgcctgcagtccctatagcctccccctgccccagtgcctttctcagctggtctgggaatcagggcagcccattgaggggggctcagacaaccctttcttcctggttctgggagtaaggctttctctgcagccacaaggagaacatgggaaccagaaacttctctttggtcagcccttctgagcacacaggtttagcccatgctccaagaatgctcctgagcattgtataagcccctaggactcttagctcatgtgataatgattgcattactatcctgctttaactgacaaaaccacagagaattttgcttgctttcctactctcttcctctgcccaggcctcctccatacgtatacatacacacatgggcatgtatacacacacatgcacaatagacatgcacacacacatgcacacatgggtatacatatacacacatgcacaatagacatgcatatatacatatacatgcacacattgataggcatacacacatgcacagtagacatgcacacatgggtatacatagacacacatgcacagacatgcatacacacacatgcacacataaatgtgcacacacacatacacaaattgcccaagcctgtgcactcctggtacccatgtcccatcctggcccacctctttgtcagcaggactgactccatcactccctcaggttttgcccaccagcaccccccagctttcccccagcagagctaaacagcccctctgaatagacaacaggaccttctagaaacagctgaacccttggacagcagcaggcagattttgatctgctctcatcaggtacaaaaggtggtatccgtattctcttctgagcatgctcagtaagcatatggacatagaatttaacatctctgtggagtgtgtgttttttacatatttgtatgcagtcgaggagggcctgttgtagaattctctccctgtatcttactatactgttaaagaagctgaattctatgttgccaacagatgcgtgaaatgttcctccaggaaaagccattcaagcctgattatttttctaagtaacttcaattaaattgaagaagaaaaaaaaaaaaaaa

SEQ ID NO: 46 - protein, 352 aa, Mus musculus
MKPALLEVMRMNRICRMVLATCFGSFILVIFYFQSMLHPVMRRNPFGVDICCRKGSRSPLQELYNPIQLELSNTAILHQMRRDQVTDTCRANSAMSRKRRVLTPNDLKHLVVDEDHELIYCYVPKVACTNWKRLMMVLSGRGKYSDPMEIPANEAHVSANLKTLNQYSIPEINHRLKSYMKFLFVREPFERLVSAYRNKFTQKYNTSFHKRYGTKIIRRQRKNATQEALRKGDDVKFEEFVAYLIDPHTQREEPFNEHWQTVYSLCHPCHIHYDLVGKYETLEEDSNYVLQLAGVSGYLKFPTYAKSTRTTDEMTTEFFQNISAEHQTQLYEVYKLDFLMFNYSVPNYLKLD

SEQ ID NO: 47 - DNA, 1785 bp, Mus musculus
  CDS (149)..(1408)
ggcgatttcggctgcagaatcagcatcaccagcaacagcagcagcggcggtgactgtggcgggcgctaggtccgtctcctagggaccatgtcccagctgtgcacaaggctgaagtgaagggccaggagtgggcccagcccagggcagcatgaccaagccgcggctcttccggctgtggctggtactagggtcggctctcatgatccttttgatcattgtatattgggacaacgtgggaaccgcccacttctatctgcacacgtctctctccaggccacacatcctagaaccccttcccacccagggattggtggaggagaacgtgttcacatctgacgtggatgagtttttggatactctccttagttctgacgcgaagcacaacgacctttccaggagaaaaactgagcagcccccggcgcccgcccccagcaagccagtcttgagccacatggaggagaacgtgagaggctacgactggtccactcatgatgcccatcagaaccctgaccgggacaggcagcaggccgagaggaggagcctgctgagagacttctgtgccaacgccagcctggcattccccaccaaggaccgctcttttgacgacatccccaactacgaactgaaccacctgatcgtggacgaccgccacggggtcatctactgctacgtgcccaaggtggcctgcaccaactggaagcgagtgatgatcgtgctgagcgagagcctgctggaccggggcagcccctaccgagaccccctggacatcccccgggaacacgtgcacaacaccagcacgcacctcaccttcaacaagttctggcgccgctacggaaagttctcccgtcacctcatgaaggtgaagctgaagaagtacaccaagttcctgttcgtgcgcgacccctttgtgcgcctcatctcagccttccgcagcaagttcgagctggagaacgaagagttttaccgcaagttcgcggtgcccatgctccgactgtacgccaaccacaccagcctgcccgcctcggtgagtgaggctttcagcgccgggctcaaggtctccttcgccaacttcatccagtacctcctagacccacacacggagaagctggcgcctttcaacgagcactggcgacaggtgtaccgcctctgccacccgtgccagatagactatgacttcgtggggaagctggagacgctcgatgaggacgctgcccagctcctgaggttcctcaaggtagactcccagctccacttcccccccagttatcggaacaggacggccagcagctgggaggaagactggtttgccaacatccccctggcatggaggcaacagctctataaactctacgaggccgactttgttctctttggctaccccaagccagaaaacctgctcagggactgagcccccagaagccctcacgctgcccccaacaaattgaatggctgtcccatgaggccgtcctttgaggatgggaccctgtggcctcctgggttctctcctggcttcctttgcttcctggtgtgacaggcagaggattccacgcccccccctcgcatctggagaccgtggtacagccaagaccgaagcacctcactctccagagttttgcgctccccacccccgcccttttgcaatctggatttgtttactccacagcctgtattcatggaacactgtgttaaatactgttttctaagattaatatatttcagatatatttaatacgaaagtgggaggaagctggagtaaagtgtggcgcccgcaaaaaaaaaaaaaaa

SEQ ID NO: 48 - protein, 419 aa, Mus musculus
MTKPRLFRLWLVLGSALMILLIIVYWDNVGTAHFYLHTSLSRPHILEPLPTQGLVEENVFTSDVDEFLDTLLSSDAKHNDLSRRKTEQPPAPAPSKPVLSHMEENVRGYDWSTHDAHQNPDRDRQQAERRSLLRDFCANASLAFPTKDRSFDDIPNYELNHLIVDDRHGVIYCYVPKVACTNWKRVMIVLSESLLDRGSPYRDPLDIPREHVHNTSTHLTFNKFWRRYGKFSRHLMKVKLKKYTKFLFVRDPFVRLISAFRSKFELENEEFYRKFAVPMLRLYANHTSLPASVSEAFSAGLKVSFANFIQYLLDPHTEKLAPFNEHWRQVYRLCHPCQIDYDFVGKLETLDEDAAQLLRFLKVDSQLHFPPSYRNRTASSWEEDWFANIPLAWRQQLYKLYEADFVLFGYPKPENLLRD

SEQ ID NO: 49 - DNA, 1728 bp, Mus musculus
  CDS (1)..(1116)
atgactgtcgcctgccacgcgtgccaggcacagcatgggaagacgctcctgttgcaggcggcccttgccggtggtggcaagtctgggtgctgcactcctgctcctgtgcgccctgcgtcccgggtaaccacaggaaaggatgcccaggacactgaatggcagggctccccaaaagcccttttgggggttccgacatttgaaaataaagctctgggctccagctggttcggtggagtgaggaagagtcccctacagctgttgcgtgacctggaccagggtccacgctccgcgatggccgaggtgcaccagcagcggcgtgagctgctgcgccgggcctgcagccgccacacgcgacgccaacgcctgctgcagccggaggacctgcgtcacgtgctggtggacgacgcgcaccggctgctgtactgctacgtgcctaaggtggcctgcaccaactggaagcgtgtgatgctggcgttgcgcggccgtggggatccaagcgcaatccctgcgcacgaggcgcatgcgcctggcctgctgccctcgctggccgactttgcgccggctgaggtcaactggcggctgcgcgactacctgacctttctcttcgtgcgggagcccttcgagcgcctggcgtcagcctaccgcaacaagctggcgcggccacacagcgcggccttccagcggcgctatggcacacgcatcgtgcgtcgcctacgaccacacgcgcagcccgatgcgctggcccgcggccacgacgtgcgcttcgccgagttcctggcctacctgctcgacccgcgcacgcgccgtcatgagcccttcaacgaacactgggagcgcgcacacgcgctgtgccatccgtgcctagtgcgctatgatgtagtgggcaagtttgagacgatagcagatgatgctgccttcgtgctggacctggtgggtgagcctgggctacgtttccctgctccaccgctgaggccagagaaggaccttacgcgtgagcaggcccggcgccttttccaggacatcagccccttctaccagcgtcgcctctttaacctctataagatggactttctgctcttcaattactctgccccttcctacctgcgactgcaataagggtgttgggtgcaatagagccagtggctgctgtgaccaggaggccaccaggaggtctggaacgaacctggttgtgtggattggagaccttatccagtgggcctgaccaagagtctggccactggtcacactcattccgactgggtagggtacaggttgctttaggtgaccataaccttgtcaggccgtttctgctgttagtttgatgtgtgtctcttcctcccactctgcagatgtcaggcttcttcctaggactccaggtttgtagttctttggtttggtttgagaggccatttctcagtcttgtctgtgtagaacctgtccgtggcatggtgctacaagacagaatgtcatggcttggttcagtgtggcctaaggtcttgtcagcatttactgcttaggagttaacatgagctgcctgccaccccagcagtcacaggatggtgagcactaccactccacatctacctggctgatctacctttgatctcagccttgcaagaggctggatcttcccctgtgtcagcaaaggccaagatgcaatactgtggcagcttttccagctcacttttattttttttgttgtttttttaaataaatatgttttgttac

SEQ ID NO: 50 - protein, 371 aa, Mus musculus
MTVACHACQAQHGKTLLLQAALAGGGKSGCCTPAPVRPASRVTTGKDAQDTEWQGSPKALLGVPTFENKALGSSWFGGVRKSPLQLLRDLDQGPRSAMAEVHQQRRELLRRACSRHTRRQRLLQPEDLRHVLVDDAHRLLYCYVPKVACTNWKRVMLALRGRGDPSAIPAHEAHAPGLLPSLADFAPAEVNWRLRDYLTFLFVREPFERLASAYRNKLARPHSAAFQRRYGTRIVRRLRPHAQPDALARGHDVRFAEFLAYLLDPRTRRHEPFNEHWERAHALCHPCLVRYDVVGKFETIADDAAFVLDLVGEPGLRFPAPPLRPEKDLTREQARRLFQDISPFYQRRLFNLYKMDFLLFNYSAPSYLRLQ

SEQ ID NO: 51 - DNA, 2073 bp, Mus musculus
  CDS (129)..(1259)
ccggccctggtccctgcctgcaccccgggagctggccacccactatccctcccctcccgagacctccagcccctgctgcagtcacctcccctgcagcctcgaggtcggcgaggtctggccgcagcaccatgtttccccgccctctgaccccactggctgccccgaaaagcgcggagaccctgggccgcacgccaaggcgggccccattgggccgggcccgggctgggctcggggggccgcccctgctgctgccgtccatgctgatgttcgctgtaatcgtggcctccagcggactgctgctcatgatcgagcgaggcatcctatcggagatgaaaccccttcccctgcaccctcccagccacaaaggcgcggcctggagcgggacagatcctaagcctagaggcctatccttggatgctggggactcggacttgcaagtgagggaggacatccgaaaccggaccttgagggccgtgtgcggacaaccaggcatgccccgggacccctgggacttgccggtgggacagcggcgcaccctgctgcgccacattctcgtaagtgaccgctaccgcttcctctactgctatgtccccaaagtggcctgctctaactggaaacgtgtgctgaaggtgctggctggcgtcctgaacaacgtggatgtccgcctcaagatggaccaccccagtgacttggtgtttctggcagacctgcggcctgaggagattcgctaccgtctgcagcactacttcaagttcctgtttgtgcgagaccccttggaacgcctcctgtctgcttaccgtaacaagtttggagagatccgagagtaccagcagcgatatggggccgaaattgtcaggcgctacagggctggagctggtcccagccctgcaggggacgatgtcaccttcccagagttcctgagatacctggtggatgaggatcctgaacatatgaatgagcattggatgcctgtgtaccacctgtgccaaccatgtgctgtgcactacgacttcgtgggttcctatgagaggctggaggctgatgccaaccaggtgctggagtgggtgcgggccccaccccatgtccggttcccagctcgccaggcctggtaccggccagccagcccagaaagtctgcattaccacttgtgcaatgttccacgggccctgcttcaagatgtgctacctaagtatatcctggacttctccctctttgcttacccactgcccaatgtcaccaaggaagcctgtcaccaatgacagtaggccagcaccttttggagtttgggtttaatgatatcagctttgggatgtctttcagagaaactcctggctctgggtggcttcctggtttctctaggtgtctccatatctcagtggtaaggactgtccttggaggtccttgtccacagtggctcagaggacagagctagaaaggaggcctgctgctttcactggtgaactgcctctcttaggggcctgtggtatccgtgtctgcagggcaccagtggttattaaagccatatgtttgatcgaaagactgacttcagccccctggctgctgggtctatgcagtccacctggtctgttgtaatttaacctgtggccaaatcccaaatatgacactagccaagcacatgatcatgcctaggaccaatggctgtgaccccctattcacccatcccatggacctcaggactggagtgagctgtggtgccttagaaatgaaatgtgtgcaattctactccagacttttacatttcctcctcttgctaggtctgaatcatttttctaaggaaagagaaacggaagtggggcccttacctcgaagctctaaagcccagcccctcaagcatccaaagacgcctgtgcctgacctcttcctagggctcctggagcatcttcaataagcctcccttccctacaaacctttggagactatgtgagactgtatggcccatatatctggctgtcacttgtctaatgcatttatttaaaatgtgtatattttaataggatccttgtaagggctgacttttaataaagctttttcatatacaaaaaaaaaaaaaaa

SEQ ID NO: 52 - protein, 376 aa, Mus musculus
MFPRPLTPLAAPKSAETLGRTPRRAPLGRARAGLGGPPLLLPSMLMFAVIVASSGLLLMIERGILSEMKPLPLHPPSHKGAAWSGTDPKPRGLSLDAGDSDLQVREDIRNRTLRAVCGQPGMPRDPWDLPVGQRRTLLRHILVSDRYRFLYCYVPKVACSNWKRVLKVLAGVLNNVDVRLKMDHPSDLVFLADLRPEEIRYRLQHYFKFLFVRDPLERLLSAYRNKFGEIREYQQRYGAEIVRRYRAGAGPSPAGDDVTFPEFLRYLVDEDPEHMNEHWMPVYHLCQPCAVHYDFVGSYERLEADANQVLEWVRAPPHVRFPARQAWYRPASPESLHYHLCNVPRALLQDVLPKYILDFSLFAYPLPNVTKEACHQ

SEQ ID NO: 53 - DNA, 6000 bp, Mus musculus
  CDS (496)..(1914)
cttagccactgaaccatctctccagcctctgttttggggtgtttgtttgagacagaatcaaaaggctggtctaaagtacatattgtagctaaagatggcttccaactcctgccttgcctccagagtgttagggtgtgacctttaaatgacctattaaagaaaccccaacaggtgtagagtacacctgtgatcccagctcttaggaggttggacagatggataaagaggtccaggtgtcccggactacatagagagaccctatctcaaaagagaggcgaagacttaggttttgagagtgggcctgaggtctctcttcaaaagttttatagaaagatgtctctgttccctgtctatgaacacggatggcctgagcacctgtctcttcacaggatcagagtgtcccccacctgaagagggctgattgggtcccaagctatgctcctgagctgagtgcctgcagccagtctgaggaactccatggcgccccctctccccatggagaaaggactcgctttgcctcaggatttccgggaccttgtacacagcctaaagattcgaggcagatacgtcttgttcctggcatttgtggtcatagtttttatcttcattgaaaaggaaaataaaatcatatccagggtctccgacaagctgaagcagatccctcattttgtggcagatgccaacagcactgacccagccctgctcttatcggagaatgcatctctcttgtccctgagcgagttggattccaccttttcccatctgcggagccgcctgcacaacctgagcctgcagctgggcgtggagccagcaatggagagccaggaggctggggcagagaagccatcccagcaggctggagcagggacccggcgccacgtgcttctcatggccaccacccgcacgggttcctcgttcgtgggcgagttcttcaaccagcagggcaatatcttctacctcttcgagccactgtggcacatcgagcgcaccgtgttcttccagcagcgaggcgccagcgcggctggttcagccttggtctaccgtgatgtcctcaagcagttgttgctatgcgacctgtatgtgctggagcccttcatcagccctccgcccgaggaccacttgactcagttcctgttccgccggggatccagccgttcactctgcgaggatccggtgtgcacacccttcgtcaagaaggtctttgagaagtaccactgcaggaaccgtcgctgcgggccactcaacgtgaccttggcgggcgaggcctgccgccgcaaggaccacgtggccctcaaggctgtgcgcatccgtcagctggagttcctgcagccgctagttgaggacccgaggttggatctacgagtcattcagctggtgcgcgacccccgggccgtgctggcttcacgcatagtggcctttgcgggcaagtatgagaactggaagaagtggctgtccgaggggcaggaccagctgagcgaggatgaggtgcagcgattgcggggcaactgtgagagcatccgcctgtctgcagagctgggcttgcggcagccagcctggctgcgcggtcgttacatgctggtgcgctatgaggatgtggcacgcaggccactgcagaaggcccgagagatgtacagctttgcgggcatccccttgaccccgcaggtggaggactggatccagaagaacacgcaggcgacacgcgacagcagcgatgtctactccactcagaaaaactcttctgagcagtttgagaagtggcgcttcagcatgcctttcaagctggcacaggtggtacaggctgcctgtggcccgaccatgcacctctttggctacaagttggccagggatgccgcctcactcaccaaccgctccatcagcctgctggaggagcggggcaccttctgggtcacgtagtgggggatgtctgggacccttggaactccttcttgtgaaaggctggccctgcttccctcacacccagcctggcagtgagacatagccctggcagaaagtcaaaatggggagcgatgatgggaacatagcccctggctgtagcggtagggccccctgccagctagactccccagagcagctacagctagggccttgggctcctctgaggacaccctgtctccttagtggtactggtcataaggtgtcctcaccctatgacctgcagtgtcccgagcaggtcaaggtaggttcctgtgtgtggacacacctccctagctctttctcacacagtctacacgaagcctgtaaaggcctgtaggttgtgtgggctggagtccaagtatttaacaaccagaaggggtgtagaccctctgcagcctctcagacctcctactgtattaagtgttaacctcttccgctctgagtcagagaagctgggatctggctgagtcctgggaaggaggaggacagcctagggtgaggaaggggacctttgaagctcctcagggaacagtggctgtgtaccagctcatagaaaatggtttgatcagctgttctagtcactcatgagtatcaatcagcctgtgtagagcaagacacacaaagtgcattgaaaacagacaaggccaggggtgtggctcagcggtaaagcccttgcctctctcatgcccagggccctaggtgtgattcttagcactataaatataagggaaaataagccatacatacgtgtgcagtacagagagagacagaaaagcgtaataaacattcccccctctcataagcacacgctctgcctctgtctctgtctctgtctctctctgtctctgtctctctctgtctctctctctcactctctctctctctctctctctctcacacacacacacacacacacacacacaaacacacacacacacactggtacagaaaagcttcccttctccactctttatgctcaccatgtttttaggatttttgtttgtttgttttgttttgggtttctatatatctaaacaaggagccttaaattacatctttttgggattacgtggcgagggggagcagcagggagcatttgcttctggtaaaaggatagaccagaaaggacttccccttggatgatcggagtcatgcagagcccccagggtcccacatgttctgtgtgaacttcatgtggaatgactcacagaagtgactaccttaggcgctcgctctgtagctcaatgtgcgtagagtttgtctattgtataggaagccctgggttctgtgccctgccccacaccatataggtatgatggcgtgtacccatatcccagcattcaggaaatcaaaggttggcggtcaaaagttcaaggtgcagctatgtatcttttgagatgtccgagtctataagctccctctactgagagctgctagtattccttctcaagaacaggttatttgaaaatctcttcttgtaagccccaacatttgggggaggggaggatgggggaactcttgactgagcagctaggtttccccaagcagaagttcttctctcccagtgcaagctgaggtttagtgccccggctagcctcggaactggctcagtgggggttggcacgctgacttcctggacaattgctgcatctgacactgtgagcgcccaggacaccggatgtgtagcatggatgtgggcctagcacaggtgtggaagaggacagtcaggacactcactgttggtgtttgttatccacctgttcagccgccgggtcccacacataggtcatctttcgtcactcaagccggcctctgtttttgattttaacaatccaagagcaagtgtgtaggggacaaagagacaccagctcttcatacttgatggagaccgggacaggatgctgcaggcaggctcgggagtgcattatcgtttcttctgagctcttcatccaggccatattcccttttctgcatcgcatctggggtgggaggggctgagctgggagtttccgtccttcttccctgtgggggtgggagatggggctcaaggtctccatctctcggctccctgaggacagaacccccacagtggacctttgggccttctcaggacattgacaatgttgtgtgcactgcaagttgactttatttattttgtaggaaaaagagatggtattctttagcaggatctgaaactgtaccctagtcaagaagtacaattaataacattattattaataacaattgggatgattcaaaggtcacacacacaccagaccacccaagaaccatgtaggagatgaggcggagccaggtcatggaagcccagaatgctacagagattctgtgggtctattattgatcagaaaatacaacatgggggggccttttctatggcataactcaggtgtctgcaaagacagagcagctcaggtgggtggataggcaggcagatggagccttgaatcacatcccctgaggctcagccagccagtgcgaatcctcaaggttcggaaagggacagtgtagcggcaaactactgggcatcttctctgatgcttagaggctcttaccaaggagcttgatggagaaagtggccatgttggtggcatgggacaactgctcacccaacttcaaagatcaaagggcacccaggtggcctatgacagtgacactctttcctaccattaggggtgtctgcccccactgaaagccatgaaatttctggccctaagggggtaggaaggacttaggagtagcagatggtttgatcccatcccccccacacacacacacacatccctcagctgtcttccacattagagccacttcagttgtccatggacttgtccctttgagacatcctggattttgaaggatagaaacatccccaagatggtctcgtgttaatcccacaacagaggcagaagggtcatactgagagagagagagagagagagagagagagagagaggagtgggggccagaccctcaagaagccaagtgggtctggtcaacctgtgcacatgagaaggagggaacatcactaaaatcagggcctgggctggtgtgttgttggtgagatcccgtggagtggctggctagatatggatgagttttctgagcatgctcacacacccccaacttcacattcttagaaatagcacaaccataatgccttacctcaaaaggatgaggcaaagcttgcaattaattcccagtgtctgaagacctccgggatcctttgagctgtgtgtccttgtcagtacatggggacaggctccttaggtattcctgacatagaggtaaggtgctgcccttgctgccttgcataagctgtgacaagcttcttactgggcacatagaacggctctgtcatctgcttccactaaatttgggcttggactttgcctcctgccaaatctccatctctgctggatagtctagtccctagggtctaaccaccaccctccacttcctggtgggtcctacaagcgctgtctttgtgccagtacccggatggtgtcctgcctcatggctaaatggtacaggacatcttcccagactgagtggtcatggcatgtgcatgtatgttcatgagtacatgcatggtgtgtgggggtggatgggtgcattctgttctgcttctggtgcagctacacagccacaacctctttctgtcattgaccttcttggtcttcttgtagccacagataatcttccaatgcccgattctgctgttctcatctgagagctgacaacccagcgctcagagtagagttcatgacctaggaaacccttctcctggtagcttactgaacttatttaattaaaaacgaacatataggggttggagagatggttcattggttaagagcaccggctgcttttgcaaaggacctgagttcaaatcccagcaaccatatggtggcttacaaccatctgtaatgagttctggtgccctcttctggcatgcaggtgtacatgcagatagagcactcctatgcataaaataaataaatcttttaaataaataaataaatgaacatgaaaaaaaaaaaaaaa

SEQ ID NO: 54 - protein, 472 aa, Mus musculus
MEKGLALPQDFRDLVHSLKIRGRYVLFLAFVVIVFIFIEKENKIISRVSDKLKQIPHFVADANSTDPALLLSENASLLSLSELDSTFSHLRSRLHNLSLQLGVEPAMESQEAGAEKPSQQAGAGTRRHVLLMATTRTGSSFVGEFFNQQGNIFYLFEPLWHIERTVFFQQRGASAAGSALVYRDVLKQLLLCDLYVLEPFISPPPEDHLTQFLFRRGSSRSLCEDPVCTPFVKKVFEKYHCRNRRCGPLNVTLAGEACRRKDHVALKAVRIRQLEFLQPLVEDPRLDLRVIQLVRDPRAVLASRIVAFAGKYENWKKWLSEGQDQLSEDEVQRLRGNCESIRLSAELGLRQPAWLRGRYMLVRYEDVARRPLQKAREMYSFAGIPLTPQVEDWIQKNTQATRDSSDVYSTQKNSSEQFEKWRFSMPFKLAQVVQAACGPTMHLFGYKLARDAASLTNRSISLLEERGTFWVT

SEQ ID NO: 55 - DNA, 2080 bp, Mus musculus
  CDS (147)..(1601)
caccgcccggctgctccactccgctccacccaacattgagggagacccgaagaggccggagccgaggactttgggctgggttttctggacagaaccagcaggcgcctactctgctctgggtggggagagtgaggattcggtgaactatgaagggccggcggcggcggcgccgaaagtattgcaagttcacgctgctcttggcgctgtacacgcttttgctacttcttgtcccctctgtactggacagccacagcgagcaggacaagggcaggaactgccccggcctgcagcgcagcttgggtgtgtggagcctggaggcggcggcggccggggaacgtgagcagggcgctgaggtgcggtccctggccgaaggaaacccggatcgatcccccgggtcccccggcaacctcagcgccgtcggtgaggcggtgacccaggaaaagcaacacatctatgtgcatgccacctggcgcaccggctcgtccttcttgggcgaactcttcaaccagcacccggacgttttctacttgtacgaccccatgtggcatctgtggcaggcactgtatccgggcgacgcggagagcctgcagggcgcactaagagacatgctgcgctccctcttccgctgtgatttctctgtgctgcgcctgtacgcgcagcctggggaccctggggagcgagcaccggactcggccaacctcaccacggccatgcttttccgctggcggaccaacaaggtcatctgctcgccgcctctgtgccccgccgcgccccgggcacgcgcggacgtgggactcgtcgaggacaaagcctgcgaaagtacctgcccgcccgtttcgctccgcgccctggaggccgagtgccgcaagtacccggtggtggtcatcaaagacgtgcggctactggacctgggagtgctggtccctctgctgcgtgacccaggcctcaacctaaaggtggtgcaactcttccgagaccctcgggccgtgcacaactcgcgcctcaagtcgaggcagggactgctgcgcgaaagcatccaggtgctgcgcacgcgccagaggggcgaccacttccaccgggtgctgctggcgcatggagtggatgcccgtccgggaggccaggcccgggctctgccctcggcgccacgcgctgatttcttcttaaccagcgcgcttgaggtgatctgtgaagcgtggcttcgcgacctgctattcacccgcggcgcgcccgcctggctgaggcgtcgctacctgcggctgcgttatgaggacctggtgtggcagccccaagcccagctgcgccgcctgctgcgcttctctgggttgcggacactcgccgcgcttgatgccttcgcattcaatatgacgcggggctcggcctacggcgccgatcgtcccttccacttgtctgcgcgggacgcccgagaggctgtgcacgcctggcgcgaacgtctgagccaagagcaggtgcgccaagtggaaaccgcctgcgcccctgccatgcgtctgcttgcctaccctcgaagtggggacgaacgcgacaggaagaccgtcagggaaggggagacaccactggagaccaaggccaattgggctgtgtaataccctgatccctgaaccctgccccggggcgtattcaggtcagtggccataaaaaggtgaactcagcatgctgcccccgcactggagaggctgcacggtggaggcgatctatcacactgtgagacactgggactgatttggtatcaactgctgtgccattctcctggtcaggagcatcacaagctgttaagtaatgacagacaccttggctgagatgaagtttccagaaaggaagtaacagtgcaatgtggatatttgtgaccacaacataggaaaagctgtacttcccaggctgaacttggctcagcttgagccatttcaacaaggcatcctcacaataatgaagagatgtgatctggtttcctttcacatcagccaagatgtctggacaaaaccatcaatgtgaataagggccaagtgcagttgtgtctctcttgattaaattacttcatattaaataaaaaaaaaaaaaaaaaaaa

SEQ ID NO: 56 - protein, 484 aa, Mus musculus
MKGRRRRRRKYCKFTLLLALYTLLLLLVPSVLDSHSEQDKGRNCPGLQRSLGVWSLEAAAAGEREQGAEVRSLAEGNPDRSPGSPGNLSAVGEAVTQEKQHIYVHATWRTGSSFLGELFNQHPDVFYLYDPMWHLWQALYPGDAESLQGALRDMLRSLFRCDFSVLRLYAQPGDPGERAPDSANLTTAMLFRWRTNKVICSPPLCPAAPRARADVGLVEDKACESTCPPVSLRALEAECRKYPVVVIKDVRLLDLGVLVPLLRDPGLNLKVVQLFRDPRAVHNSRLKSRQGLLRESIQVLRTRQRGDHFHRVLLAHGVDARPGGQARALPSAPRADFFLTSALEVICEAWLRDLLFTRGAPAWLRRRYLRLRYEDLVWQPQAQLRRLLRFSGLRTLAALDAFAFNMTRGSAYGADRPFHLSARDAREAVHAWRERLSQEQVRQVETACAPAMRLLAYPRSGDERDRKTVREGETPLETKANWAV

SEQ ID NO: 57 - DNA, 512 bp, Mus musculus
atgaaaggcatcttatggatgtgctcaaccttattactaacccatgcactacatcaagccaaaatggaaaccagcccacctgttaaaggctctctgtctggaaaagtggtcctaccttgtcatctttcaaccttacctaccttaccacccaattacaacacgagtgaatttctcagaatcaaatggtctaagatggaagtggacaaaaatggaaaagatataaaggagacgactgtcttggtggcccagaacggaaatatcaagattggtcaggactacaaggggcgagtgtccgtgcctacacatcccgatgatgtaggtgatgcttccctcaccatggtcaaactccgggctagtgatgcaggcgtctaccgatgtgatgtcatgtatgggattgaagacactcaggacaccatgtcactggctgtggatggtgttgtgtttcactacagggcagccaccagcaggtacactctgaactttgccgctgctcaacaggcttgtttggatat

SEQ ID NO: 58 - DNA, 536 bp, Mus musculus
acaaagccatgaagccggcgctgctggaagtgatgaggatgaacagaatttgccggatggtgctggccacttgcttcggatcctttatcttggtcatcttctatttccaaagtatgttgcacccagtcatgcggaggaaccccttcggtgtggacatctgctgccggaagggatcgagaagtcccctgcaggagctctacaatcccatccagctggagctatccaacactgccatcctgcaccagatgagacgggaccaggtgacagacacctgccgggccaacagtgccatgagccgcaagcgcagggtgctgacccccaacgacctgaagcacctggtggtggatgaggaccacgaactcatctactgctatgtgcccaaggtagcgtgcaccaactggaagaggctcatgatggtcctgagtggccggggcaagtacagcgatcccatggagatcccagccaacgaagcccacgtgtcggccaacctgaagacccttaaccagtacagcatcccagagatcaaccaccgcttg

SEQ ID NO: 59 - DNA, 660 bp, Mus musculus
gccaggagtgggcccagcccagggcagcatgaccaagccgcggctcttccggctgtggctggtactagggtcggctctcatgatccttttgatcattgtatattgggacaacgtgggaaccgcccacttctatctgcacacgtctctctccaggccacacatcctagaaccccttcccacccagggattggtggaggagaacgtgttcacatctgacgtggatgagtttttggatactctccttagttctgacgcgaagcacaacgacctttccaggagaaaaactgagcagcccccggcgcccgcccccagcaagccagtcttgagccacatggaggagaacgtgagaggctacgactggtccactcatgatgcccatcagaaccctgaccgggacaggcagcaggccgagaggaggagcctgctgagagacttctgtgccaacgccagcctggcattccccaccaaggaccgctcttttgacgacatccccaactacgaactgaaccacctgatcgtggacgaccgccacggggtcatctactgctacgtgcccaaggtggcctgcaccaactggaagcgagtgatgatcgtgctgagcgagagcctgctggaccggggcagcccctaccgagaccccctggacatcccccgggaaca

SEQ ID NO: 60 - DNA, 600 bp, Mus musculus
atgactgtcgcctgccacgcgtgccaggcacagcatgggaagacgctcctgttgcaggcggcccttgccggtggtggcaagtctgggtgctgcactcctgctcctgtgcgccctgcgtcccgggtaaccacaggaaaggatgcccaggacactgaatggcagggctccccaaaagcccttttgggggttccgacatttgaaaataaagctctgggctccagctggttcggtggagtgaggaagagtcccctacagctgttgcgtgacctggaccagatgtttggcagctgtgagctatgggtagtcagtggggagcggcagaaagtgggtccacgctccgcgatggccgaggtgcaccagcagcggcgtgagctgctgcgccgggcctgcagccgccacacgcgacgccaacgcctgctgcagccggaggacctgcgtcacgtgctggtggacgacgcgcaccggctgctgtactgctacgtgcctaaggtggcctgcaccaactggaagcgtgtgatgctggcgttgcgcggccgtggggatccaagcgcaatccctgcgcacgaggcgcatgcgcctggcctgctgccctcgctggcc

SEQ ID NO: 61 - DNA, 600 bp, Mus musculus
gcgccccctctccccatggagaaaggactcgctttgcctcaggatttccgggaccttgtacacagcctaaagattcgaggcagatacgtcttgttcctggcatttgtggtcatagtttttatcttcattgaaaaggaaaataaaatcatatccagggtctccgacaagctgaagcagatccctcattttgtggcagatgccaacagcactgacccagccctgctcttatcggagaatgcatctctcttgtccctgagcgagttggattccaccttttcccatctgcggagccgcctgcacaacctgagcctgcagctgggcgtggagccagcaatggagagccaggaggctggggcagagaagccatcccagcaggctggagcagggacccggcgccacgtgcttctcatggccaccacccgcacgggttcctcgttcgtgggcgagttcttcaaccagcagggcaatatcttctacctcttcgagccactgtggcacatcgagcgcaccgtgttcttccagcagcgaggcgccagcgcggctggttcagccttggtctaccgtgatgtcctcaagcagttgttgctatgcgacctgtat

SEQ ID NO: 62 - DNA, 720 bp, Mus musculus
tggggagagtgaggattcggtgaactatgaagggccggcggcggcggcgccgagagtattgcaagttcacgctgctcttggcgctgtacacgcttttgctacttcttgtcccctctgtactggacagccacagcgagcaggacaagggcaggaactgccccggcctgcagcgcagcttgggtgtgtggagcctggaggcggcggcggccggggaacgtgagcagggcgctgaggtgcggtccctggccgaaggaaacccggatcgatcccccgggtcccccggcaacctcagcgccgtcggtgaggcggtgacccaggaaaagcaacacatctatgtgcatgccacctggcgcaccggctcgtccttcttgggcgaactcttcaaccagcacccggacgttttctacttgtacgagcccatgtggcatctgtggcaggcactgtatccgggcgacgcggagagcctgcagggcgcactaagagacatgctgcgctccctcttccgctgtgatttctctgtgctgcgcctgtacgcgcagcctggggaccctggggagcgagcaccggactcggccaacctcaccacggccatgcttttccgctggcggaccaacaaggtcatctgctcgccgcctctgtgccccgccgcgccccgggcacgcgcggacgtgggactcgtcgaggacaaagcctgcgaaagtacctgcccgcccgtttcgc

SEQ ID NO: 63 - DNA, 600 bp, Mus musculus
accccacaactcggaacgatgcggctagcctgcatgttctcgtccatcctgctgtttggagctgcgggcctgctcctcttcatcagcctccaggaccctatagagctcagcccccagcaagttccaggtataaagttcagcatcaggccccagcaaccccagcatgatagccacttgaggatatccacagaaaagggcacacgagattcacccagcgggtcgccaagaggcctccagctgcaagcgcctgaccaacctcgacctcacccgaaggcagcgggatctcctttgcgcctccggcagcgcaggcggagactgctcatcaaaaagatgccagccgcagggactaaccaaggcaacaactcgtccgaaacctttatccagccgagaccccgcaccatggacagtcgttgggtcagcctgcaccagacccaacaggagcgcaagcgtgtgatgcgagaagcctgcgctaaatacagggccagcagcagccgcagagctgtcactccccgccacgtctcccgcatcttcgtggaggaccgccaccgtgtactgtactgtgaagtacccaaggcaggctgctccaactggaagagggtg

SEQ ID NO: 64 - DNA, 735 bp, Mus musculus
atatagtatctaggatatatgtagaagacaaacacaaaatcctgtactgtgaagtaccaaaagctggctgctctaattggaaaagaattctgatggtcctaaatggattggcttcctctgcatacaatatctcccatgatactgtgcactatgggaagcatctgaaaacactggatagttttgacttaaaaggagtacacatgcgtttgaatacatataccaaagctgtgtttgttagagatcccatggaaagattagtctccgcatttagggataaatttgagcatcccaatagttactaccatccggtgtttggaaaggcaattatcaagaaatatcgaccaaatgcctctgcagaagcattaaataatggatctggagtcaaattcaaagaattcgcctactatttgctggatgctcaccgtccagtaggaatggatattcactgggaaagagtcagcaaactgtgttatccgtgtttgatcaactatgactttgtagggaagtttgagaccttaggagaggatgccaattactttctacagttgattggtgctccaaaagagttgacatttccaaactttaaggataggcactcctctgatgaaagaaccaatgcccacgtggtaaggcagtatttaaaggacctgagcacagccgaaagacagctcatctatgacttctatcacttggactatttgatgtttaattacacaactccacatttgtaatttg

SEQ ID NO: 65 - DNA, 574 bp, Mus musculus
ccagaagccaagctcattgttatgctcagggatcctgtggagaggttgtactcagactatctctacttcgcaagttccaataaatctgcagatgacttccacgagaaagtgacggaggctctgcagctgtttgaaaattgcatgctggattattcactgcgcgcctgcgtctacaacaacaccctgaacaatgccatgcccgtgaggctccaggttggcctgtatgctgtgtacctcctggattggctgactgtcttcagtaaggagcagttcctcattctccgtctggaagaccacgcatccaatgtcaagtataccatgcacaaggtcttccagtttctaaacctggggcctctgagtgagaagcaggaagctctgatgaccaagagccctgcatccaacacacggcgtcctgaggatcgtagtctgggacccatgtggcccatcacccagaagattctgcgtgaattctatgggcctttcaacacaaggctggcacaggtcttggatgacgaagcatttgcctggaagacaacgtgagagctgagtcccttctgcagaagctgggcccact

SEQ ID NO: 66 - DNA, 1430 bp, Mus sp.
tgcgacttcatcaagtcggagccaaagaaatcttgaaagatgggaatgctagcaagagttgccctgggacttatcatcattgatgctgtattggctgcaccaactacagagctctttaactatgactcagaagtgtatgatgccatcttggaggacacagggaccttttataactatgaacacattcctgacaaccatgtagagaacgagaaagtgtctgagaggctttctgggaacagagagctcctgaccccaggcccacagctaggggacaaccaggatgaagacaaggatgaagaatctactcccaggctgatcgatggctcttccccacaggagccagaattcccggggcttctgggtccacacaccaacgaagactttccaacttgtctcctgtgtacttgtataagtaccactgtatactgtgatgaccatgaactggacgctattcctccactgcccaagaagactacatatttctattcacgttttaacagaattaaaaagatcaacaaaaatgactttgcaagcttaaatgatttaaaaaggatcgatctgacatcaaatttaatatcggagattgatgaagatgcattccgaaagctgcctcacctccaagagctggttctgcgtgacaataagataaagcagcttccagaattgccaaacactttgacatttattgatatcagcaacaatagactgggaagaaaaggcattaaacaagaagcttttaaagatatgtatgatctccaccatctctatatcaccgataacagcttggaccacatccctctaccactcccagaaagtctgcgggctcttcacctccagaataatgacattctggagatgcatgaagatactttctgcaatgttaaaaatttaacttatgttcgtaaggcattagaggatattcgactggatggaaaccctatcaacctcagcagaactcctcaagcctacatgtgtctacctcgtttgcccattgggagttttatctaatgttaggatgctaggcaaatatcagaattgtgatgctttctgtagccaagagaagcatatatctcttcagaagcaaactcagtatatttgagatgcgtttaaaacatgtggcacaatgatataaacaataagctaaaggtgttgagataaaataagagttcaataatttaattaaacatgattaggtataaattaacaaaatatatagacagtaagttaaataaaatgtatgccaactagtcatcaaatctcatcaagaattgtaggtgttcatgtcaagttaaatatgtaagaaataaaatattggtaatgaatactttagatacgttgctaagatgtagacattttaattaaacttctctagtctttattttcactcatgtatgtttcccacctgattcagttaagagcagaaaaaaata

SEQ ID NO: 67 - DNA, 26 bp, Mus musculus
accccacaactcggaacgatgcggct

SEQ ID NO: 68 - DNA, 24 bp, Mus musculus
tgcatgttctcgtccatcctgctg

SEQ ID NO: 69 - DNA, 26 bp, Mus musculus
cgccaccgtgtactgtactgtgaagt

SEQ ID NO: 70 - DNA, 25 bp, Mus musculus
aggctgctccaactggaagagggtg

SEQ ID NO: 71 - DNA, 24 bp, Mus musculus
atatagtatctaggatatatgtag

SEQ ID NO: 72 - DNA, 25 bp, Mus musculus
gaagtaccaaaagctggctgctcta

SEQ ID NO: 73 - DNA, 26 bp, Mus musculus
ttctatcacttggactatttgatgtt

SEQ ID NO: 74 - DNA, 25 bp, Mus musculus
tacacaactccacatttgtaatttg

SEQ ID NO: 75 - DNA, 24 bp, Mus musculus
ccagaagccaagctcattgttatg

SEQ ID NO: 76 - DNA, 25 bp, Mus musculus
ctgtggagaggttgtactcagacta

SEQ ID NO: 77 - DNA, 26 bp, Mus musculus
atttgcctggaagacaacgtgagagc

SEQ ID NO: 78 - DNA, 27 bp, Mus musculus
gtcccttctgcagaagctgggcccact

SEQ ID NO: 79 - DNA, 25 bp, Mus musculus
gcgccccctctccccatggagaaag

SEQ ID NO: 80 - DNA, 25 bp, Mus musculus
gctttgcctcaggatttccgggacc

SEQ ID NO: 81 - DNA, 27 bp, Mus musculus
ggttcagccttggtctaccgtgatgtc

SEQ ID NO: 82 - DNA, 25 bp, Mus musculus
gcagttgttgctatgcgacctgtat

SEQ ID NO: 83 - DNA, 24 bp, Mus musculus
tggggagagtgaggattcggtgaa

SEQ ID NO: 84 - DNA, 27 bp, Mus musculus
cggacgtgggactcgtcgaggacaaag

SEQ ID NO: 85 - DNA, 26 bp, Mus musculus
cgaaagtacctgcccgcccgtttcgc

SEQ ID NO: 86 - DNA, 9120 bp, Mus musculus
tgtatatctgagtatctgtgtatgtgcatatatgtgtgtctatgtgtatgtgtgggtttgtgcatgtgtgtctgtgtgtacatgtctgtgtgtctctgtgtgtgtctgtgtatgtgtgtttacatgtgtgtgtgtgtttctgtgtggacgcatgtgtatgtgtctccgtgtgcctgcatgtgtgtttgtgtatgtgtgtaggcatgagtacatcatggtgtacatgtagaaattgagagagactcttgggtgtggttctcatctcccaccatatctgaagtaaatgtcttattcacaccactgtgcacatcagaaagctagccttgacctccaaggaactctgtctctgtctcctgtcttattgtaggtacctggggtttataagcctatgctgctgcatatggcttcacttgggttctggggatttgaacttaggtcctcacagttttagggcaagcattttattcactgaacaatctcccatgatccctagagtttattgtaggcaacttaacctttagtcgtcattgagatttagcattgatttcagttccagttgtattctgatgttgagcgctacagcagaggtacccagggactgctgattttctgtggccatgaatcctctagtgattaataaatttcatcataatgttctgtttatttctaaatgtccagacaactctaatttcaaattaatctcatcctaaacagcacaggataaaagttacagattgtcctatcttcattcatgtgatgcctcaaatcagatgtttaatcctttagtcagttatttgtgaaacggaacctaattacacacactcgcatgcgcgtgcgcgcacacacacacacacacacacacacacacacacacacacacacttgtagatttgcccacacagctgcttaagcaatttggagcaaatgtagaaatattatgaaatttcacataaaaatgggttttcctctgtcttcttgagaaatgtgtgagtttcaatatgacagtaatcttctggtgttggactataaccacttgaacccctgaggtactaagtgtgtacctactacgtgcctggattccatcatttccctacttatataagcttctccatcctgtagatcagcactgtgtaatggagtaacacaagtcattgaatcatgcaatcatttaatcaatttaattggcacttctgataagagaagtgggcacatatttagttgtgtttgcagcctgtgctatgtcatttttctctatatatactttaagatttttttcttttttccatttacggggggggggttattccatttaatttacatgcttaatgacatataatttgcatatcaggtgttcctttctttagagttaacttcttttaggaccattgtttataatggatgtatatcatccctaaattaaatcagccattgtctacttaacacaccagtttctctgatgtccctctggctcttcctcctctgaggttctagttataggaaagtctggcattttgatgtttttccatggcttcgtgggctctgatcatccacttacagtgtactttcatctcttgtctacaaactgcatatcatctattgatctgcgttcaaggctactacattttcatgcaattccactttgctgttaaaccttttctatgatccgtttttatacttataaacaatccaagtgttgtactctttgggtataaatgttctgtatttcacaggtcttaacttcttttcagactatacaaagaaagattttctattttttttatttgttgtatgcattttatgcatttagcactgggatttccataacaggcagttaagatcattgaatacatactttgtgtggtaaatataatacggcttatttgattctcccccttaaaatgctcccatcttctcagtcatgtcaggttgctatggagtccaccctgagcaccatgagtgtgatgctgtacagacactcctgatatgccttctgttatttgaactccacctgcaagttttcattcaagcatagctcctaatcccattcagtgtgcacttttcttaattgatttgctggtagagagctatatgtaagcatggcttagcatcaaattgaactttctcctctccctagcttctttccagtatattcttcctcaccttcatcagattttgaggtcttgagatctaaaccttcatgttacaatttgcagagataccaggatctctatcagacttgatgtgtctccatgtaattctagtatcagcctttcataagataattgcaaaatatagaaccccccctctctccttttgactattacagctttgtttctctcccaagttaatacagatgtatgcaaatcaactaagaaaagcaaacactgaatgggataaggagctatactcaaaagaaagcttgcagttggagttcaaataacacaagtggaaccaagagtatctgcacagcatcagactcactgtgctcaggatggaatccatagcaacctcccataactgagaggatgtgagccccttttaaagggaaaatcaaataagcagtattatattaaccacatgatgtgtatattcaatgtagattgggctgcaccgaagttatgattttagtaatgagtatttctccttgggttttcttctggaccaccctggttttccttttaaaaatagtaaaatagcagcaatgactgcttctcttaaaatctgaacaacacagagcacttcagcatcctttcaacctatctaaatccattgtagacctgcactgacttgctgtgagctattgtgtcacaactattattgtagctaaacaccttgaattttcaaagctgctctggactgtgtcttcttttagaattaaacttgctaaagatattggactctgagatgcttattcaccactttctataaaagagtaaggatgacaatgctttgagtagaagaagattgcatacgagggtaccagcatgtcattatgtccccagcagtcagtaaagtaaggatccagtgatggtagagttattatgtccattatggaactaatgtaaatgtttatcaatgactagaatggctgtaatcaccaatccttagacaagtgtgagtaacatcatgctcttttcttataataatagatagtcacacacaccatggtcttaaacattacattttgatttttctctttgtgtctgtcttttcgtacctctactccacttacctgagccttcagctagggctcctccaagacggcagtcacagtactagtgaggataattctcatatggagacctgaagcagaataaccttcttccaaggaaaccaaaatcctagtcaacatcttctccactctttggttatttatttttatttctatttctattttgccatttaagaatttttaaactcttttctgttttcttatatatattcttttttttacaacttttgatattctttatcaaatgatttatcccattgcctttcctcctgcagctatctgtgagttttagcatggatatttagaatacaattggaaattatgaaggtcgaataaaagttctctaagagccaggacctcactagcctagttagttgcttagtttctaatataaggcatggtttatctcctactgtatggcccttaaatccactagacatctgttggcttccaacagcatacaattgctgctattgtatgtttaggtatatatttttatgaaacactatattctgattggtgttcattcttcctatgaaggggaagattttattgcctgctcaaattcaagtcagtcctttttgagtagacaaaggttgtccaatcttgctaaacccttttcttttttgcttggggaatttctttagggaaaattctcaaccatgaaccaagcctagagattgataatagctgaaggcagagtagccagtcttagtaagggatgagagaaagactgaccaaagactgaataggaaatggttgatagcattcattgggactagacaataaagttagctaccccaacatgggcaaatggagaaaatggggagagtcatatggatatgtagggaggagaagaaaggaaggggtgagtttgaagtatggaagagcctttaaagcatttgtaagatttgtaagaaaaagcttttgagagacagagtattacctgccttcaaatcccttcaattactgtccctacaagacatacctgtgctcagctgctcttggaccacaagggggcctaagagcatgtttgtgtccctgtctttatacaggcttctcatctgtatttctcaaacgtaaatacaaatgggggagataacaaatgatatttctaagtggtacacaggtatgagaactgcacacaatctatttatatgtataatggccagagctctcagcagggaacagaggaagcctaggtaactcctgctccagagcatgtctgtatggagacctcagttcagcaactatgaaatctgtgttcaaagggtcatcactgggaacagtttctggaatagcagacatattcattctctctctctctctctctctctctctctctctctctctctctctctctctctctctctgtctgtctctgtctctctctgtctgtctgtctgtctctcccttctgtcaggggtatgcatacatgggctgggaattctatcttccctgcctgctgtctttggtgtctccctaatgtggtgccatgtttacttaacctgtctgaaatggaggataacctctggtctgataactgtcaagttactgaaatgtcagggaattgtgaatgaacccctctgaatgctgggcagatgttttgagcacgtgggtatccatagatttttttttcagcaggatttccttctttaatatattgtgggaacgaaacattgaagtttccaacactaagaaaagcaatgctctgggtttgcaaagaagtctccaaagctgtgggagaggcaatggctttgcctcacaaaggtgatcctgatttggattgggtgttcctcagtctccctcatcctttgtttgtggaccaaacctctgatcaaataatcagtatacagacaagtactgactgactcctgtcactgtttcccatgtaaattctcaaccatgtgtgtcagaattgtttgagtcatcaaagttccgtgatgctcaggatggtgaataccagaaactgaagtctgaaatccaatttggcctttctcgggcactgtaaactgagtgcaaatgtgcccagttccctcgtggcccatttatagcacaataacacagtgcttgcttacaaaatctagtcccgattaaagtatttatttaaagttgctttagacaaatatatttctaaatggaaggatgaaacagaaagaatattccaactgtacagtctatattttgtttctaaaaatttttattagaattctgacttttagaaaacaaatagatattcaaaaactacttctttaattaaataataacatcattatagacattctccagccatttagaagtagcatgattacaaagcattcatgttccctttgttttttgaaacagaagtatttatatgacaaataaaaagcaaaaatctcatatatgtgatagctatagatgcattgataggaattaaatttaatcaaaaagaacttttaatggcaataatgtggctactaacttcattgtttgtaataatgctgcaaatgtttaatgaaaactgttactaacaatacaggagaagattacagatatgattaggtcgtgttgatagaaatggaaagagaaaggtcactcactacctcagccacagaacttgaggaatgtgtgagggtttttgaggaaaaaagagaacaatgtaacatcacagcaacatcttaatctgaaagtagacgacaaaggtttcagagtaggggatcagaaatttaagtatgcagagaagcaaggcatatacggcttagacaatgtatgaagaacagcagcagacagcaagtgaaagaaatcacagtccacatggaaatcacacagaccacagtgtgcacacagagacagaaatcagcaagggacacacggcttccagttatgaaagttagacagcaaaatttaagttgtcccacctaaaacatgatgtatcgtctctcaaatggggaaggggaaggagcagcaaacatctggatatgtctaaaacctcacagagaacatctgggatctttgcatttctccctctgactttctcaatatgttagctacttctagatagtttcaataatggtctctaattgactatggagagacaggtctcatgtatgtgaacatccttaaccaggagatggaatgtgtttccttgtcactcattttctaaggaagtgaattccttctgcattgcccaggattggatgttacgtgaaactaaaaatcattgattaaaaagaaatgtcattaccacagttgggttgaatcaatcaagacttatcctagttacaggcatcacctccccttaagcacaaatactgagaacagctaagctcactttgaaatctgggatgaggaggctttaggtcaagcatctgacaatgtgtgtgtgtatgtgtgtgtgtgtgtgtgtgtgtagtatgattatatgtatatatgtagatgtgtgtatatatttatatgtatatatgtatatgtgagtatgtatatatgtatatgtgtgtatgtgtatgagtgtgtgtatgtatgactatgagtgtgtatgtattgtgcaagtgtgtgtatatatgtgtgtatatgtttatgtatatgtgtattgtatgggtgtgaatgtattatatatgtgtgtaggtacatatgtgtatatgtttatgcgtgtatatgtgtatgcattatatgtatgtgtgtatgtgtgtatgttttcatgtgtatgtagcatatgtatatgcatgtgtgtgtctgtgtgtgtaagcatgtatttactaagaaaaaatcagtttctctagctaatgagtcattgaagtgaagaaaccttacgctatcacattactatgtttccagcataaattgcaatatcagtaagacattccccatcataacttaatggacacttagattaggtcatgcagcagtaaattatgaaagcgtctatgctgagctgaggtgggaaatcgtctttcctgcctccttcctcctgttctttctcctaaaggccctcatcctctttctccccttcctttcttttaaaatattctatcattgatccaagctaactaacccttcccttgctcacatgtgacctgcccagaggaatctatttatagtcaacaataacaggccaaggacatgcgaacaaatgactttacttagccccacttggtgaatctattgggtgaataggtaaggggttatttataggagcttgactgactggcacagctgtatctctgaaaagtcccccaaagcatcatgggtgggaactaaggaaagccggaaatgctggagctggaatcccaacctgcagtggctacatgatgaatgtgtctcctttcctactaaatatttaagactctgggcaggctggttagtcttgtaaaagtcttgtgggtttctggagtcctgggaggctcactgtccctgcagaagcttgttattattgtatatgaaagaaagtgctcaacactgagtcacaaaagaagcccccgagaatcggtggcagaggtggaaatagaagaatccgatgcagttcccgaggcaagagttgatcctctccatggtgacaagaaaacaagagccacagaagcctgaaggctaagactatgacctgtgaggttctcctctcctaggacttgaatgcacttaactctgagggctagcagggcagagattcaggagcgttctcagaggttgtcccttctccttgtatgattctaggagaagctcagtgcataggcagaagttgcaaactctaacacattaacccacatcctgaattgttttatatcaattgttttataactacatgttttaacttttgttttggtttttgaattttgtttgtttgtttgttttctatttttcatatgtgtgcatgtgagccagaagacaggctaagggactctgttctctctttccagcatgtgggtttcagggatcaaagttaggtcccaaggcttcactgggcaacgggacccttagcctctgagtcatcttcttaatctttttctttgaaactttttcattacattgtgtcagtgcacacacacacacacacacacatgcatatacatccacatgcatatacatccacatgccatatgtcatggcacatgtgtggaaagaaatgggtgccttgaggaaatcagttttcttcttccatcacgtggatcttatgacttcaggtaacccatctctgctccccaagcatggctacaagcatttacctgccaagtcatctccctggctcccacattaatttataataatacaccctttcagcctagagagatgactcagtggttaagaactctggctgtgcttcagaaggttctgagttcaattcccagcagctataaggtggctcacaactatctctaatgttgttttctgtcctcttctggcatgcacttgtatgtgcagatagagtacttatatgcataaataaatataaatacattaataatcttttaaaaagaaggaaagattaaaagaacaatacaccctttcttttaagtaagaaaacatgcttagtatatagaataaatgctggtgttgaaaggtgaaattcagaagaaattataggttggaaatatttgaactcttttgtgtcaaaccaaatggtaaagatgatgtgataataaggtaaggttttcttcctacacatatacttgtttaataaccagtataagactatgagtgaacataactaaaaatacatgaaaaaaaactattttaaaaaaacatcactttcagatgactctggtgaaactcactgataaccagcaatctctttgatagaccagagtgtccagtctgaagttaattcttttgctttattctcaaacaatgttgcagaagtatgaaccatagtatcaagatacactgtccacatatgcacctggcctgacttgtgt

(19) United States
(12) Patent Application Publication
(10) Pub. No.: US 2009/0202514 A1
(43) Pub. Date: Aug. 13, 2009

(54) AGENTS FOR IMPROVING CHRONIC OBSTRUCTIVE PULMONARY DISEASES

(76) Inventors: Hiroyuki Yoneyama, Tokyo (JP); Jun Koyama, Tokyo (JP); Yoshirou Kai, Tokyo (JP)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/576,042

(22) PCT Filed: Nov. 28, 2006
(86) PCT No.: PCT/JP2006/323664

§ 371 Date: Feb. 28, 2008

(30) Foreign Application Priority Data
Aug. 25, 2006 (JP) 2006-229312

(51) Int. Cl. A61K 38/47; C12Q 1/34; C12N 9/24

(52) U.S. Cl. 424/94.61; 435/18; 435/200

(57) ABSTRACT

The present invention relates to agents for suppressing pulmonary emphysematous lesions and agents for suppressing emphysematous lesions which are suitable for treating and/or preventing COPD, which comprise as an active ingredient a substance having an activity of promoting CSPG degradation, inhibiting CSPG synthesis, or inhibiting CSPG sulfation (examples of such agents are: chondroitinase ABC, C6ST antisense agents, and GalNAc antisense agents); agents for treating and/or preventing COPD; methods for suppressing emphysematous lesions; and methods for treating and/or preventing COPD.

### TECHNICAL FIELD

The present invention relates to agents for suppressing the deposition of chondroitin sulfate proteoglycans (CSPG) in lung tissues and agents for suppressing pulmonary emphysematous lesions, which comprise as an active ingredient a substance having an activity of promoting CSPG degradation, inhibiting CSPG synthesis, or inhibiting CSPG sulfation; methods for suppressing the deposition of CSPG; methods for suppressing emphysema formation; and

treatment or prevention of pulmonary emphysema or chronic obstructive pulmonary disease (COPD) using these methods.

The present invention relates to agents for suppressing pulmonary emphysematous lesions and agents for suppressing emphysematous lesions which are suitable for treating and/or preventing COPD, which comprise as an active ingredient a substance having an activity of promoting CSPG degradation, inhibiting CSPG synthesis, or inhibiting CSPG sulfation (representative examples of such agents are: chondroitinase ABC, C6ST antisense agents, and GalNAc antisense agents); agents for treating and/or preventing COPD; methods for suppressing emphysematous lesions; and methods for treating and/or preventing COPD.

### BACKGROUND ART

The chronic obstructive pulmonary disease (COPD) is a group of diseases with chronic and persistent obstructive ventilatory impairment which frequently occurs in middle-aged smokers. The World Health Organization (WHO) statistics indicate that COPD is the fourth leading cause of death. In Japan, COPD was the tenth leading cause of death in 2000 (Non-patent Document 1). In Japan, the Japanese Respiratory Society Guideline (Non-patent Document 1) defines COPD as a disease characterized by obstructive ventilatory impairment induced by the onset of either or both of pulmonary emphysema (PE) and chronic bronchitis (CB). Of the two, PE is the major target of the present invention, and the number of its potential patients is estimated to be about 3 million people, including people with milder pathological conditions.

The pathological conditions of PE are defined as conditions pathologically characterized by the destruction of terminal bronchioles, their distal airways, and alveolar walls, and irreversible enlargement of the air space, with no clear sign of fibrosis or airway obstruction. It is thought that the underlying mechanism of the onset of PE can be interpreted by the “elastase-antielastase imbalance hypothesis” or in terms of alveolar wall apoptosis (Non-patent Documents 2 and 3); however, many points are yet to be clarified. The most obvious intrinsic risk factor is the hereditary disease a1 -antitrypsin deficiency; however, the disease is very rare in Japan (Non-patent Document 1).

Recent reports on COPD described that the levels of cytokines, such as tumor necrosis factor (TNF-α), macrophage chemotactic protein (MCP)-1, macrophage inflammatory protein (MIP)-1β, transforming growth factor (TGF)-β, interleukin (IL)-1, IL-6, and IL-8, all of which were in vivo inflammatory substances associated with alveolar macrophages and neutrophils in expiratory concentrates, bronchoalveolar lavage fluids (BAL fluids) and airway tissues (Non-patent Documents 4 to 7). These cytokines are assumed to be produced by airway epithelial cells stimulated by tobacco smoke or other factors, as well as by alveolar macrophages and neutrophils attracted to local sites in the lungs. Suppressing the migration of immune cells into airways and alveolar regions is thought to be a key to COPD treatment.

The formation of pulmonary emphysema, which is caused by various factors described above, is irreversible. It would be difficult to treat pulmonary emphysema using current therapeutic methods other than lung transplantation. Thus, there is a worldwide need to develop a novel therapeutic agent that inhibits the progression of emphysema formation and enhances the regeneration and repair of lesions. Studies aiming at the suppression or treatment of pulmonary emphysema using matrix metalloproteinase inhibitors or the like have been conducted recently, but they are not yet clinically applicable (Non-patent Documents 8 and 9). Furthermore, vascular endothelial growth factor (VEGF) has been used as an attempt to treat pulmonary emphysema, in which tissue repair is enhanced through increased neovascularization using VEGF; however, it is still in the research stage.

Meanwhile, proteoglycan, the target substance of the present invention, is a general name for molecules having the structure with one or more glycosaminoglycan (GAG) chains covalently linked to a protein called core protein, and is a component of the cell surface or extracellular matrix (Non-patent Documents 10 and 11). The specific sugar chain structure of GAG chains linked to a proteoglycan is believed to be involved in various functions of the proteoglycan. Based on the type of GAG backbone structure, proteoglycans are roughly divided into four groups: chondroitin sulfate proteoglycan, dermatan sulfate proteoglycan, heparan sulfate proteoglycan, and keratan sulfate proteoglycan (Non-patent Documents 12 to 18). Of them, heparan sulfate proteoglycan has been extensively studied, because its function is strongly modulated through binding with various cytokines, adhesion molecules, and chemokines. There are reports that indicate a correlation of PE onset with decrease of heparan sulfate proteoglycan (Non-patent Documents 19 and 20).

Meanwhile, chondroitin sulfate proteoglycans are essential molecules in the fetal period and they are abundant in each organ, but they are known to decrease along with birth, growth, and aging. However, the in vivo functions of chondroitin sulfate proteoglycans remain to be clarified. In recent years, the finding that mice genetically deficient in CSPG show embryonic lethality and organogenesis failure has raised the awareness that CSPG is an essential molecule in the body.

Furthermore, a report on PE and CSPG indicates that the onset of PE is correlated with aging-related decrease of CSPG in lung tissues (Non-patent Document 21). In addition, Rao et al. have reported that PE was suppressed by administering CSPG-comprising sulfated polysaccharides to PE model hamsters; the model is prepared by using human leukocyte-derived elastase (Non-patent Document 22). However, the correlation between CSPG and the onset of PE remains unclear.
[Non-patent Document 1] “Guidelines for the Diagnosis and Treatment of COPD (1999)”, ed., The editing committee on The Japanese Respiratory Society COPD Guidelines
[Non-patent Document 2] Hoshino, Y., Am J Physiol Lung Cell Mol Physiol. (2001) 281(2): 509-16
[Non-patent Document 3] Aoshiba, K., Am J Respir Cell Mol Biol. (2003) 28(5): 555-62
[Non-patent Document 4] Am J Respir Crit Care Med. 1996 Feb. 153(2): 530-4
[Non-patent Document 5] Am Rev Respir Dis. 1987 Sep. 136(3): 779-82
[Non-patent Document 6] Eur Respir J. 1999 Jul. 14(1): 160-5
[Non-patent Document 7] J Pathol. 2000 Apr. 190(5): 619-26
[Non-patent Document 8] Selman, M., Am J Physiol Lung Cell Mol Physiol. 2003 Nov, 285(5): 1026-36
[Non-patent Document 9] Funada, Y., Kobe J. Med. Sci. (2004) 50(3): 59-67
[Non-patent Document 10] Berfield, M., Annu Rev Biochem. (1999) 68: 729-777
[Non-patent Document 11] Iozzo, R. V., Annu Rev Biochem. (1998) 67: 609-652
[Non-patent Document 12] Lindahl, U. et al., (1972) In Glycoproteins (Gottschalk A. ed) 491-517, Elsevier, New York
[Non-patent Document 13] Oegema, T. et al., J. Biol. Chem. (1984) 259: 1720-1726, 15
[Non-patent Document 14] Sugahara, K. et al., J. Biol. Chem. (1988) 263: 10168-10174
[Non-patent Document 15] Sugahara, K. et al., J. Biol. Chem. (1992) 267: 6027-6035
[Non-patent Document 16] De Waard. et al., J. Biol. Chem. (1992) 267: 6036-6043
[Non-patent Document 17] Moses, J. Oldberg et al., Eur. J. Biol. (1992) 248: 521-526, 19
[Non-patent Document 18] Yamada, S. et al., Trends in Glycoscience and Glycotechnology (1998) 10: 95-123
[Non-patent Document 19] van Straaten, J. F., Mod Pathol. 1999 Jul. 12(7): 697-705
[Non-patent Document 20] van Kuppevelt, T. H., Am J Respir Cell Mol Biol. 1997 Jan. 16(1): 75-84
[Non-patent Document 21] Konno, K., Am Rev Respir Dis. 1982 Nov. 126(5): 797-801
[Non-patent Document 22] Rao, N. V., Am Rev Respir Dis. 1990 Aug. 142(2): 407

### DISCLOSURE OF THE INVENTION

### Problems to be Solved by the Invention

An objective of the present invention is to provide obstructive ventilatory impairment-suppressing agents, therapeutic agents against chronic obstructive pulmonary diseases that comprise the above agents as active ingredients, and methods of screening for obstructive ventilatory impairment-suppressing agents.

### Means to Solve the Problems

The present inventors conducted dedicated studies to develop such agents. They suspected that excess accumulation of CSPG, which was not considered as a conventional cause of pulmonary emphysematous lesions, might enhance the destruction of lung tissues or inhibit tissue repair and thereby contributes to the onset and exacerbation of pulmonary emphysematous lesions. The present inventors conducted studies based on this assumption and as a result, discovered that chondroitinase ABC efficiently degraded the CSPG accumulated in pulmonary emphysematous lesions and administration of chondroitinase ABC to subjects significantly suppressed emphysema formation in lung tissues. In addition, the present inventors discovered that clinical symptoms of the respiratory function that accompany pulmonary emphysematous lesions were improved in vivo, by chondroitinase ABC and gene therapy agents including agents that suppress the gene expression of C6-sulfotransferase which is essential for in vivo synthesis of CSPG (C6ST antisense agents), and agents that suppress the gene expression of C4-sulfotransferase and C6-sulfotransferase which act on N-acetylgalactosamine (GalNAc), a component of CSPG (GalNAc antisense agents). Thus, the inventors completed the present invention.

The present invention relates to obstructive ventilatory impairment-suppressing agents, therapeutic agents for chronic obstructive pulmonary diseases that comprise the above agents as active ingredients, and methods of screening for obstructive ventilatory impairment-suppressing agents. More specifically, the present invention provides:

[1] An obstructive ventilatory impairment-suppressing agent which comprises as an active ingredient a substance that inhibits the production or accumulation of a chondroitin sulfate proteoglycan.
[2] The agent of [1], wherein the substance has an activity of promoting the degradation of a chondroitin sulfate proteoglycan.

[3] The agent of [1], wherein the substance has an activity of inhibiting the synthesis of a chondroitin sulfate proteoglycan.

[4] The agent of [1], wherein the substance has an activity of desulfating a chondroitin sulfate proteoglycan.
[5] The agent of [1], wherein the substance has an activity of inhibiting the sulfation of a chondroitin sulfate proteoglycan.
[6] The agent of any of [1] to [5], wherein the production or accumulation of a chondroitin sulfate proteoglycan is inhibited in a lung.
[7] The agent of any of [1] to [6], which is used for treating or preventing a chronic obstructive pulmonary disease.
[8] The agent of [7], wherein the chronic obstructive pulmonary disease is pulmonary emphysema.
[9] The agent of [7], wherein the chronic obstructive pulmonary disease is chronic bronchitis.
[10] A method of screening for an obstructive ventilatory impairment-suppressing agent, which comprises selecting from a test sample a substance with an activity of inhibiting the production or accumulation of a chondroitin sulfate proteoglycan.
[11] The method of [10], which comprises the step of selecting a substance with the activity of any of:
(a) promoting the degradation of a chondroitin sulfate proteoglycan;
(b) inhibiting the synthesis of a chondroitin sulfate proteoglycan;
(c) desulfating a chondroitin sulfate proteoglycan; and
(d) inhibiting the sulfation of a chondroitin sulfate proteoglycan.
[12] The method of [10] or [11], wherein the obstructive ventilatory impairment-suppressing agent is used for treating or preventing a chronic obstructive pulmonary disease.

Further, the present invention relates to the followings:

[13] Use of the agent of any of [1] to [9] for producing an obstructive ventilatory impairment-suppressing agent.
[14] A method of treating a chronic obstructive pulmonary disease which comprises the step of administering the agent of any of [1] to [9] to a subject (such as patients).
[15] A composition which comprises the agent of any of [1] to [9] and a pharmaceutically acceptable carrier.

### EFFECT OF THE INVENTION

An objective of the present invention is to provide agents for suppressing pulmonary emphysematous lesions based on the mechanisms of promoting CSPG degradation, inhibiting CSPG synthesis, desulfating CSPG, and inhibiting CSPG sulfation to suppress the deposition of CSPG in lung tissues, and emphysematous lesion-suppressing agents that are suitable for treating and/or preventing COPD; agents for treating and/or preventing COPD; methods for suppressing emphysematous lesions; and methods for treating and/or preventing COPD.

The present invention demonstrated that the production and accumulation of chondroitin sulfate proteoglycans were involved in the onset of obstructive ventilatory impairment and that its onset was suppressed by inhibiting the production and accumulation of chondroitin sulfate proteoglycans. Thus, it is possible to provide new-concept therapeutic agents for chronic obstructive pulmonary diseases. In particular, the number of potential patients with pulmonary emphysema, a chronic obstructive pulmonary disease, is about 3 million in Japan. Therefore. such new-concept therapeutic agents have great medical and industrial significances.

### BRIEF DESCRIPTION OF THE DRAWINGS

FIG. 1 (upper panel) is a set of light microscopy images of pulmonary emphysema model mice obtained by hematoxylin-eosin staining (HE staining). The emphysema was induced by intratracheal administration of porcine pancreatic elastase (PPE). The magnification was 50 fold. The lower-panel graph shows the average interalveolar distance in the upper-panel photographs. The distance was measured for evaluating the degree of emphysematous lesion.

The control group (PPE(−), Chase ABC(−)); the untreated group (PPE(+), Chase ABC(−)); enzyme-treated group (PPE(+), Chase ABC(+)). *P<0.01 (t test)

FIG. 2 is a set of photographs showing the effect of reducing CSPG deposition (brown signal) in the enzyme-treated group of the intratracheal PPE administration-induced pulmonary emphysema model mice. The magnification was 50 fold.

FIG. 3 is a set of photographs showing the effect of reducing the accumulation of F4/80-positive alveolar macrophages (brown signal) in the enzyme-treated group of the intratracheal PPE administration-induced pulmonary emphysema model mice. The magnification was 50 fold.

FIG. 4 is a graph comparing the amounts (μg/Lt lung) of glycosaminoglycans (GAG) deposited in the left lungs of the intratracheal PPE administration-induced pulmonary emphysema model mice. The amounts were compared among five groups: control group, untreated group, enzyme-treated group, C6ST-treated group (PPE(+), C6ST antisense agent), and GalNAc-treated group (PPE(+), GalNAc antisense agent). *P<0.01, **P<0.05 (t test).

FIG. 5 is a graph showing the static lung compliance (Cst) in the intratracheal PPE administration-induced pulmonary emphysema model mice. Cst is an indicator of lung elasticity. The Cst values were compared among five groups: control group, untreated group, enzyme-treated group, C6ST-treated group, and GalNAc-treated group. *P<0.01 (t test).

FIG. 6 is a graph comparing the right lung volumes (μl) of intratracheal PPE administration-induced pulmonary emphysema model mice. The volumes were compared among five groups: control group, untreated group, enzyme-treated group, C6ST-treated group, and GalNAc-treated group. *P<0.01 (t test).

### BEST MODE FOR CARRYING OUT THE INVENTION

The present invention is specifically described below:

Pathological conditions associated with pulmonary emphysema, a representative chronic obstructive pulmonary disease, include destruction of terminal bronchioles, their distal airways, and alveolar walls, and irreversible enlargement of the air space. The present inventors focused on the functions of chondroitin sulfate proteoglycans in order to establish that the improvement of legions in a lung is an effective therapeutic method for pulmonary emphysema. The inventors then suppressed the accumulation of chondroitin sulfate proteoglycans in a mouse model, and closely analyzed this condition to reveal that legion was improved (such as abolishment of alveolar septal walls and alleviation of emphysematous lesions) and the accumulation of chondroitin sulfate proteoglycans decreased in tissues. Specifically, the inventors discovered that inhibiting the production or accumulation of chondroitin sulfate proteoglycans facilitated the amelioration of abnormal accumulations of chondroitin sulfate proteoglycans in lung tissues, a factor deeply involved in pulmonary emphysema, and thus leading to the improvement of legion.

The present invention relates to chronic obstructive pulmonary disease-suppressing agents, which comprise substances that inhibit the production or accumulation of chondroitin sulfate proteoglycans as active ingredients.

The present invention is specifically described below:

In the present invention, “chondroitin sulfate proteoglycans” are a type of proteoglycan and collectively refer to compounds in which proteins (core proteins) are covalently linked to chondroitin sulfate/dermatan sulfate, which are representative sulfated mucopolysaccharides. In the present invention, preferable “chondroitin sulfate proteoglycans” are human chondroitin sulfate proteoglycans. The species from which the proteoglycans are derived are not particularly limited. Proteins of nonhuman organisms (homologues, orthologues, and such) that are equivalent to the chondroitin sulfate proteoglycans are also included in the chondroitin sulfate proteoglycans of the present invention. For example, the present invention can be conducted as long as the species has a protein corresponding to a human chondroitin sulfate proteoglycan and equivalent to a human chondroitin sulfate proteoglycan. Furthermore, the chondroitin sulfate proteoglycans in the present invention also include so-called part-time proteoglycans, which are temporarily linked with glycosaminoglycan (GAG) chains to become proteoglycans upon inflammation or the like.

Examples of the chondroitin sulfate proteoglycans described below are aggrecan, versican, neurocan, brevican, β-glycan, decorin, biglycan, fibromodulin, and PG-Lb. However, the chondroitin sulfate proteoglycans in the present invention are not limited to these examples, and may be any substances with a chondroitin sulfate proteoglycan activity. Herein, chondroitin sulfate proteoglycan activities include, for example, cell adhesion ability and cell growth promotion. Those skilled in the art can assay chondroitin sulfate proteoglycan activities by assaying cell division and growth of tumor cells (for example, Caco-2 and HT-29 cells) in the presence of a protein containing a partial amino acid sequence of a chondroitin sulfate proteoglycan, or a protein with high homology to such a partial amino acid (typically 70% homology or higher, preferably 80% or higher, more preferably 90% or higher, and most preferably 95% or higher). Proteins with the effect of promoting cell division and growth can be evaluated as proteins with a chondroitin sulfate proteoglycan activity (Int. J. Exp. Pathol, 2005 Aug. 86(4), 219-29; and Histochem Cell Biol, 2005 Aug. 124(2), 139-49). High homology means 50% or higher homology, preferably 70% or higher homology, more preferably 80% or higher homology, and still more preferably 90% or higher homology (for example, 95% or higher homology, or 96%, 97%, 98%, 99% or higher homology). Such homology can be determined using the mBLAST algorithm (Altschul et al., Proc. Natl. Acad. Sci. USA, 1990, 87, 2264-8; Karlin and Altschul, Proc. Natl. Acad. Sci. USA, 1993, 90, 5873-7).

Herein, “obstructive ventilatory impairment” means a condition where abnormalities are observed in a series of ventilation processes carried out by the respiratory system such as lung and bronchus. It includes, for example, destruction of airways and alveolar walls, and enlargement of air space, but is not limited thereto.

Herein, “inhibition of production or accumulation” of chondroitin sulfate proteoglycans includes, for example, “promotion of degradation”, “inhibition of synthesis”, “desulfation”. and “inhibition of sulfation” of chondroitin sulfate proteoglycans; however, “inhibition of production or accumulation” is not limited thereto, and it refers to a reduction or loss of the amount, function or activity of a chondroitin sulfate proteoglycan, as compared to a comparison subject. Herein, “substances that inhibit the production or accumulation” of chondroitin sulfate proteoglycans are not particularly limited. Such substances are preferably “substances with the effect of promoting the degradation of chondroitin sulfate proteoglycans”, “substances with the effect of inhibiting the synthesis of chondroitin sulfate proteoglycans”, “substances with the effect of desulfating chondroitin sulfate proteoglycans”, or “substances with the effect of inhibiting the sulfation of chondroitin sulfate proteoglycans”.

“Promotion of degradation” of chondroitin sulfate proteoglycans includes, for example, inhibition of the expression of core proteins of chondroitin sulfate proteoglycans, and a reduction in the abundance of the core proteins. Herein, “core proteins of chondroitin sulfate proteoglycans” include, for example, aggrecan, versican, neurocan, and brevican for matrix-type chondroitin sulfate proteoglycans; and β-glycan, decorin, biglycan, fibromodulin, and PG-Lb for membrane chondroitin sulfate proteoglycans. Those described above are all examples, but the core proteins are not limited to these and a wide variety of proteins may serve as chondroitin sulfate proteoglycan cores.

“Expression” includes “transcription” from genes, “translation” into polypeptides, and “suppression of degradation” of proteins. The “expression of core proteins of chondroitin sulfate proteoglycans” refers to transcription and translation of the genes encoding core proteins of chondroitin sulfate proteoglycans, or production of core proteins of chondroitin sulfate proteoglycans through transcription and translation. Furthermore, the “function of core proteins of chondroitin sulfate proteoglycans” includes, for example, their function in binding to chondroitin sulfate, and in binding with other cellular components. Those skilled in the art can appropriately evaluate (measure) the various above-mentioned functions using general methods. Specifically, the evaluation can be performed using the methods described in the Examples herein below, or using the same methods with appropriate modifications.

The “promotion of degradation” of chondroitin sulfate proteoglycans may also be an increase in the expression of enzymes that cleave or degrade chondroitin sulfate proteoglycans, or of enzymes involved in the cleavage or degradation of proteoglycans. Such enzymes include, for example, metalloproteinases (for example, ADAMTS-1, ADAMTS-4, and ADAMTS-5), chondroitinase, and calpain I, but are not limited thereto. The “promotion of degradation” may be a reduction in the abundance of chondroitin sulfate proteoglycans caused by administering all or some of the enzymes.

Alternatively, “promotion of degradation” may be achieved by administering a substance that promotes the suppression of expression of chondroitin sulfate proteoglycans. Such substances include, for example, n-butylate, diethylcarbamazepine, tunicamycin, non-steroidal estrogen, and cyclofenil deiphenol, but are not limited thereto.

Preferred embodiments of the “substance with the activity of promoting degradation” include, for example, compounds (nucleic acids) selected from the group consisting of:

(a) antisense nucleic acids against transcripts of the genes encoding the core proteins of chondroitin sulfate proteoglycans, or portions thereof,
(b) nucleic acids with the ribozyme activity of specifically cleaving transcripts of genes encoding core proteins of chondroitin sulfate proteoglycans; and
(c) nucleic acids with the activity of using RNAi effect to inhibit the expression of genes encoding core proteins of chondroitin sulfate proteoglycans.

Furthermore, the “substances with the activity of promoting degradation” include, for example, compounds selected from the group consisting of:

(a) antibodies that bind to core proteins of chondroitin sulfate proteoglycans;
(b) chondroitin sulfate proteoglycan variants that are dominant-negative for core proteins of chondroitin sulfate proteoglycans; and
(c) low-molecular-weight compounds that bind to core proteins of chondroitin sulfate proteoglycans.

“Inhibition of synthesis” of chondroitin sulfate proteoglycans includes, for example, inhibition of biosynthesis of glycosaminoglycans and inhibition of enzymes involved in the synthesis of chondroitin sulfate proteoglycans, but is not limited thereto. The inhibition refers to inhibition of any of the processes of chondroitin sulfate proteoglycan synthesis.

As substances that inhibit the synthesis of chondroitin sulfate proteoglycans, substances inhibiting the biosynthesis of glycosaminoglycans include, for example, β-D-xyloside, 2-deoxy-D-glucose (2-DG), ethane-1-hydroxy-1,1-diphosphonate (ETDP), and 5-hexyl-2-deoxyuridine (HudR). Such substances inhibit the biosynthesis of glycosaminoglycans, and thereby inhibit the synthesis of chondroitin sulfate proteoglycans.

Meanwhile, enzymes involved in chondroitin synthesis include, for example, GalNAc4ST-1, GalNAc4ST-2, GALNAC4S-6ST, UA2OST, GalT-I, GalT-II, GlcAT-I, and XylosylT. The synthesis of chondroitin sulfate proteoglycans is inhibited by inhibiting such enzymes, suppressing the expression thereof, or the like.

Preferred embodiments of “substances with the activity of inhibiting synthesis” include, for example, compounds (nucleic acids) selected from the group consisting of:

(a) antisense nucleic acids against transcripts of genes encoding chondroitin sulfate proteoglycan synthetases, or portions thereof;
(b) nucleic acids with the ribozyme activity of specifically cleaving transcripts of the genes encoding chondroitin sulfate proteoglycan synthetases; and
(c) nucleic acids with the activity of using RNAi effect to inhibit the expression of genes encoding chondroitin sulfate proteoglycan synthetases.

The “substances with the activity of inhibiting synthesis” also include, for example, compounds selected from the group consisting of:

(a) antibodies that bind to chondroitin sulfate proteoglycan synthetases;
(b) chondroitin sulfate proteoglycan synthetase variants that are dominant-negative for chondroitin sulfate proteoglycan synthetases; and
(c) low-molecular-weight compounds that bind to chondroitin sulfate proteoglycan synthetases.

The “desulfation” of chondroitin sulfate proteoglycans refers to the removal of a sulfate group from chondroitin sulfate proteoglycans, and includes, for example, desulfation by an endogenous or exogenously-administered desulfation enzyme, and suppression of sulfation by a sulfation-suppressing compound, but is not limited thereto. “Desulfation” refers to the process of sulfate group removal.

Such desulfation enzymes include, for example, chondroitin-4-sulfatase and chondroitin-6-sulfatase. Sulfation-suppressing compounds include, for example, chlorate and EGF receptor antagonists.

Preferred embodiments of such “substances with desulfating activity” include, for example, compounds (nucleic acids) selected from the group consisting of:

(a) antisense nucleic acids against transcripts of genes encoding proteins that suppress chondroitin sulfate proteoglycan-desulfating enzymes, or portions thereof;
(b) nucleic acids with the ribozyme activity of specifically cleaving transcripts of the genes encoding proteins that suppress chondroitin sulfate proteoglycan-desulfating enzymes; and
(c) nucleic acids with the activity of using RNAi effect to inhibit the expression of genes encoding proteins that suppress chondroitin sulfate proteoglycan-desulfating enzymes.

The “substances with desulfating activity” also include, for example, compounds selected from the group consisting of:

(a) antibodies that bind to compounds that suppress chondroitin sulfate proteoglycan-desulfating enzymes;
(b) variants of proteins that suppress chondroitin sulfate proteoglycan-desulfating enzymes, which are dominant-negative for proteins that suppress chondroitin sulfate proteoglycan-desulfating enzymes; and
(c) low-molecular-weight compounds that bind to compounds that suppress chondroitin sulfate proteoglycan-desulfating enzymes.

Herein, “desulfation-suppressing compounds” include not only proteins but also non-pertinacious compounds such as coenzymes.

The “activity of inhibiting sulfation” of chondroitin sulfate proteoglycans includes, for example, inhibition of sulfotransferases, but is not limited thereto. The activity refers to the inhibition of sulfation in the process of chondroitin sulfate proteoglycan synthesis.

Such sulfotransferases include, for example, chondroitin

D-N-acetylgalactosamine-4-O-sulfotransferase 1 (C4ST-1), chondroitin
D-N-acetylgalactosamine-4-O-sulfotransferase 2 (C4ST-2), chondroitin

### D-N-acetylgalactosamine-4-O-sulfotransferase 3 (C4ST-3), D4ST, C6ST-l, and C6ST-2.

Preferred embodiments of “substances with the activity of inhibiting sulfation” include, for example, compounds (nucleic acids) selected from the group consisting of:

(a) antisense nucleic acids against transcripts of genes encoding sulfotransferases for chondroitin sulfate proteoglycans, or portions thereof;
(b) nucleic acids with the ribozyme activity of specifically cleaving transcripts of the genes encoding sulfotransferases for chondroitin sulfate proteoglycans; and
(c) nucleic acids with the activity of using RNAi effect to inhibit the expression of genes encoding sulfotransferases for chondroitin sulfate proteoglycans.

The “substances with the activity of inhibiting sulfation” also include, for example, compounds selected from the group consisting of:

(a) antibodies that bind to sulfotransferases for chondroitin sulfate proteoglycans;
(b) sulfotransferase variants for chondroitin sulfate proteoglycans; and
(c) low-molecular-weight compounds that bind to sulfotransferases for chondroitin sulfate proteoglycans.

The above enzymes shown as examples include not only single enzymes that correspond to single genes, but also groups of enzymes that share certain characteristics. For example, chondroitinase is a collective name for enzymes such as ABC, AC, and B, whose substrate specificities or such are different, but which share the characteristics of mucopolysaccharide-degrading enzymes. For example, chondroitinase AC I eliminatively cleaves the N-acetylhexosaminide linkages of chondroitin sulfates (A, C, and E), chondroitin. chondroitin sulfate-dermatan sulfate hybrids, and hyaluronic acid, and yields oligosaccharides with Δ4-glucuronate residues at the non-reducing ends. This enzyme does not act on dermatan sulfate (chondroitin sulfate B. which has L-iduronic acid for a hexuronic acid), keratan sulfate, heparan sulfate, and heparin. Meanwhile, chondroitinase AC II eliminatively cleaves the N-acetylhexosaminide linkages of chondroitin, chondroitin sulfate A, and chondroitin sulfate C, and yields Δ4-unsaturated disaccharides (ΔDi-0S, ΔDi-4S, and ΔDi-6S). This enzyme also acts well on hyaluronic acid. The enzyme does not act on dermatan sulfate (chondroitin sulfate B), which is therefore a competitive inhibitor of the enzyme. Chondroitinase B (dermatanase) eliminatively cleaves N-acetylhexosaminide linkages to L-iduronic acids in dermatan sulfate, and yields oligosaccharides (di- and tetra-saccharides) with Δ4-hexuronate residues at the non-reducing ends. This enzyme acts on neither chondroitin sulfate A nor chondroitin sulfate C, which are free of L-iduronic acid. Dermatan, which is a derivative of dermatan sulfate in which the sulfate group is removed, does not serve as a substrate for this enzyme. This enzyme preferentially cleaves portions of dermatan sulfate in which the second of the L-iduronic acid units are sulfated. Chondroitinase ABC eliminatively cleaves N-acetylhexosaminide linkages of chondroitin sulfate A, chondroitin sulfate C, dermatan sulfate, chondroitin, and hyaluronic acid, and yields mainly disaccharides with Δ4-hexuronate groups at the non-reducing ends. This enzyme does not act on keratan sulfate, heparin, and heparan sulfate. Chondroitinases collectively refer to enzymes sharing the characteristics of mucopolysaccharide-degrading enzymes while also having different characteristics as described above, and they are not limited to chondroitinase ACI, chondroitinase AC II, chondrotinase B, and chondroitinase ABC as exemplified above.

Further, on a genomic DNA level, such groups of enzymes sharing features do not necessarily correspond to single genes. For example, both chondroitin-4-sulfatase and chondroitin-6-sulfatase can be retrieved from the public gene database Genbank as sequences referred to by multiple accession numbers (for example, Genbank accession Nos: NT_{—}039500 (a portion thereof is shown under accession No: CAAA01098429 (SEQ ID NO: 86)), NT_{—}078575, NT_{—}039353, NW_{—}001030904, NW_{—}001030811, NW_{—}001030796, and NW_{—}000349).

The above example proteins that correspond to single genes are shown below: Specifically, below are the accession numbers in the public gene database Genbank, nucleotide sequences, and amino acid sequences for human genes encoding:

aggrecan, versican, neurocan, brevican, β-glycan, decorin, biglycan, fibromodulin, and PG-Lb, which are shown above as examples of chondroitin sulfate proteoglycans; ADAMTS-1, ADAMTS-4, ADAMTS-5, and calpain I, which are shown above as examples of enzymes that cleave or degrade chondroitin sulfate proteoglycans or related enzymes; GalNAc4ST-1, GalNAc4ST-2, GALNAC4S-6ST, UA20ST, GalT-I, GalT-II, GlcAT-I, and XylosylT, which are shown above as examples of enzymes involved in chondroitin synthesis; C4ST-1, C4ST-2, C4ST-3, D4ST, C6ST-1, and C6ST-2, which are shown above as examples of sulfotransferases.
Aggrecan (Accession No: NM_{—}007424; nucleotide sequence: SEQ ID NO: 1; amino acid sequence: SEQ ID NO: 2)
Versican (Accession No: BC096495; nucleotide sequence: SEQ ID NO: 3; amino acid sequence: SEQ ID NO: 4)
Neurocan (Accession No: NM_{—}010875; nucleotide sequence: SEQ ID NO: 5; amino acid sequence: SEQ ID NO: 6)
Brevican (Accession No: NM_{—}007529; nucleotide sequence: SEQ ID NO: 7: amino acid sequence: SEQ ID NO: 8)
β-glycan (Accession No: AF039601; nucleotide sequence: SEQ ID NO: 9; amino acid sequence: SEQ ID NO: 10)
Decorin (Accession No: NM_{—}007833; nucleotide sequence: SEQ ID NO: 11; amino acid sequence: SEQ ID NO: 12)
Biglycan (Accession No: BC057185; nucleotide sequence: SEQ ID NO: 13: amino acid sequence: SEQ ID NO: 14)
Fibromodulin (Accession No: NM_{—}021355; nucleotide sequence: SEQ ID NO: 15; amino acid sequence: SEQ ID NO: 16)
PG-Lb (Accession No: NM_{—}007884; nucleotide sequence: SEQ ID NO: 17; amino acid sequence: SEQ ID NO: 18)
ADAMTS-1 (Accession No: NM_{—}009621; nucleotide sequence: SEQ ID NO: 19; amino acid sequence: SEQ ID NO: 20)
ADAMTS-4 (Accession No: NM 172845; nucleotide sequence: SEQ ID NO: 21; amino acid sequence: SEQ ID NO: 22)
ADAMTS-5 (Accession No: AF140673; nucleotide sequence: SEQ ID NO: 23; amino acid sequence: SEQ ID NO: 24)
Calpain I (Accession No: NM_{—}007600; nucleotide sequence: SEQ ID NO: 25; amino acid sequence: SEQ ID NO: 26)
GalNAc4ST-1 (Accession No: NM_{—}175140; nucleotide sequence: SEQ ID NO: 27; amino acid sequence: SEQ ID NO: 28)
GalNAc4ST-2 (Accession No: NM_{—}199055; nucleotide sequence: SEQ ID NO: 29; amino acid sequence: SEQ ID NO: 30)
GALNAC4S-6ST (Accession No: NM_{—}029935; nucleotide sequence: SEQ ID NO: 31; amino acid sequence: SEQ ID NO: 32)
UA20ST (Accession No: NM_{—}177387; nucleotide sequence: SEQ ID NO: 33; amino acid sequence: SEQ ID NO: 34)
GalT-I (Accession No: NM_{—}016769; nucleotide sequence: SEQ ID NO: 35: amino acid sequence: SEQ ID NO: 36)
GalT-II (Accession No: BC064767; nucleotide sequence: SEQ ID NO: 37; amino acid sequence: SEQ ID NO: 38)
GlcAT-I (Accession No: BC058082; nucleotide sequence: SEQ ID NO: 39; amino acid sequence: SEQ ID NO: 40), or Accession No: NM_{—}024256; nucleotide sequence: SEQ ID NO: 41; amino acid sequence: SEQ ID NO: 42)
XylosylT (Accession No: NM_{—}145828; nucleotide sequence: SEQ ID NO: 43; amino acid sequence: SEQ ID NO: 44)
C4ST-1 (Accession No: NM_{—}021439; nucleotide sequence: SEQ ID NO: 45; amino acid sequence: SEQ ID NO: 46)
C4ST-2 (Accession No: NM_{—}021528; nucleotide sequence: SEQ ID NO: 47; amino acid sequence: SEQ ID NO: 48)
C4ST-3 (Accession No: XM_{—}355798; nucleotide sequence: SEQ ID NO: 49; amino acid sequence: SEQ ID NO: 50)
D4ST (Accession No: NM_{—}028117; nucleotide sequence: SEQ ID NO: 51; amino acid sequence: SEQ ID NO: 52)
C6ST-1 (Accession No: NM_{—}016803; nucleotide sequence: SEQ ID NO: 53; amino acid sequence: SEQ ID NO: 54)
C6ST-2 (Accession No: AB046929; nucleotide sequence: SEQ ID NO: 55; amino acid sequence: SEQ ID NO: 56)

In addition to the proteins listed above, the proteins of the present invention include those exhibiting high homology (typically 70% or higher, preferably 80% or higher, more preferably 90% or higher, and most preferably 95% or higher) to sequences shown in the Sequence Listing and with a function of the proteins listed above (for example, the function of binding to intracellular components). The proteins listed above are, for example, proteins comprising an amino acid sequence with an addition, deletion, substitution, or insertion of one or more amino acids in any of the amino acid sequences of SEQ ID NOs: 2, 4, 6, 8, 10, 12, 14, 16, 18, 20, 22, 24, 26, 28, 30, 32, 34, 36, 38, 40, 42, 44, 46, 48, 50, 52, 54, and 56, in which the number of altered amino acids is typically 30 amino acids or less, preferably ten amino acids or less, more preferably five amino acids or less, and most preferably three amino acids or less.

The above-described genes of the present invention include, for example, endogenous genes of other organisms which correspond to DNAs comprising any of the nucleotide sequences of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55 (homologues to the human genes described above, or the like).

Each of the endogenous DNAs of other organisms which correspond to DNAs comprising any of the nucleotide sequences of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55 are generally highly homologous to a DNA of any of SEQ ID NOs: 1, 3, 5, 7, 9, 11, 13, 15, 17, 19, 21, 23, 25, 27, 29, 31, 33, 35, 37, 39, 41, 43, 45, 47, 49, 51, 53, and 55. High homology means 50% or higher homology, preferably 70% or higher homology, more preferably 80% or higher homology, and still more preferably 90% or higher homology (for example, 95% or higher, or 96%, 97%, 98%, or 99% or higher). Homology can be determined using the mBLAST algorithm (Altschul, et al. Proc. Natl. Acad. Sci. USA, 1990, 87, 2264-8; Karlin and Altschul, Proc. Natl. Acad. Sci. USA, 1993, 90, 5873-7). When the DNAs have been isolated from the body, each of them may hybridize under stringent conditions to a DNA of SEQ ID NO: 1, 3, 5, 7, 9, 11, 13, 15, 17, 19, 21, 23, 25, 27, 29, 31, 33, 35, 37, 39, 41, 43, 45, 47, 49, 51, 53, or 55. Herein, stringent conditions include, for example, “2× SSC, 0.1% SDS, 50° C.”, “2× SSC, 0.1% SDS, 42° C.”, and “1× SSC, 0.1% SDS, 37° C.”; more stringent conditions include “2× SSC, 0.1% SDS, 65° C”, “0.5× SSC, 0.1% SDS, 42° C.”, and “0.2× SSC, 0.1% SDS, 65° C.”.

Those skilled in the art can appropriately obtain proteins functionally equivalent to the above-described proteins from the above-described highly homologous proteins by using methods for assaying the activity of promoting the degradation of CSPGs, inhibiting the synthesis of CSPGs, desulfating CSPGs, or inhibiting the sulfation of CSPGs. Specific methods for assaying the activities are described below in a section on the screening methods of the present invention. Further, based on the nucleotide sequences of the above-described genes, those skilled in the art can appropriately obtain endogenous genes of other organisms that correspond to the above-described genes. In the present invention, the above-described proteins and genes in non-human organisms, which correspond to the above-described proteins and genes, or the above-described proteins and genes that are functionally equivalent to the above-described proteins and genes, may simply be referred to using the above-described names.

The proteins of the present invention can be prepared not only as natural proteins but also as recombinant proteins using genetic recombination techniques. The natural proteins can be prepared by, for example, methods of subjecting cell extracts (tissue extracts) that may express the above-described proteins to affinity chromatography using antibodies against the above-described proteins. On the other hand, the recombinant proteins can be prepared, for example, by culturing cells transformed with DNAs encoding the proteins described above. The above-described proteins of the present invention can be suitably used, for example, in the screening methods described herein below.

In the present invention, “nucleic acids” refer to both RNAs and DNAs. Chemically synthesized nucleic acid analogs, such as so-called “PNAs” (peptide nucleic acids), are also included in the nucleic acids of the present invention. PNAs are nucleic acids in which the fundamental backbone structure of nucleic acids, the pentose-phosphate backbone, is replaced by a polyamide backbone with glycine units. PNAs have a three-dimensional structure quite similar to that of nucleic acids.

Methods for inhibiting the expression of specific endogenous genes using antisense technology are well known to those skilled in the art. There are a number of causes for the action of antisense nucleic acids in inhibiting target gene expression, including: inhibition of transcription initiation by triplex formation;

transcription inhibition by hybrid formation at a site with a local open loop structure generated by an RNA polymerase;
transcription inhibition by hybrid formation with the RNA being synthesized:
splicing inhibition by hybrid formation at an intron-exon junction;
splicing inhibition by hybrid formation at the site of spliceosome formation;
inhibition of transport from the nucleus to the cytoplasm by hybrid formation with mRNA:
splicing inhibition by hybrid formation at the capping site or poly(A) addition site:
inhibition of translation initiation by hybrid formation at the translation initiation factor binding site;
inhibition of translation by hybrid formation at the ribosome binding site adjacent to the start codon;
inhibition of peptide chain elongation by hybrid formation in the translational region of mRNA or at the polysome binding site of mRNA; and
inhibition of gene expression by hybrid formation at the protein-nucleic acid interaction sites. Thus, antisense nucleic acids inhibit the expression of target genes by inhibiting various processes, such as transcription, splicing, and translation (Hirashima and Inoue, Shin Seikagaku Jikken Koza 2 (New Courses in Experimental Biochemistry 2), Kakusan (Nucleic Acids) IV: “Idenshi no Fukusei to Hatsugen (Gene replication and expression)”, Ed. The Japanese Biochemical Society, Tokyo Kagakudojin, 1993, pp. 319-347).

The antisense nucleic acids used in the present invention may inhibit the expression and/or function of genes encoding any of the CSPG core proteins, synthetases, proteins suppressing desulfation enzymes, and sulfotransferases described above, based on any of the actions described above. In one embodiment, antisense sequences designed to be complementary to an untranslated region adjacent to the 5′ end of an mRNA for a gene encoding an above-described CSPG core protein, synthetase, desulfation enzyme-suppressing protein, or sulfotransferase may be effective for inhibiting translation of the gene. Sequences complementary to a coding region or 3′-untranslated region can also be used. Thus, the antisense nucleic acids to be used in the present invention include not only nucleic acids comprising sequences antisense to the coding regions, but also nucleic acids comprising sequences antisense to untranslated regions of genes encoding the above-described CSPG core proteins, synthetases, desulfation enzyme-suppressing proteins, or sulfotransferases. Such antisense nucleic acids to be used are linked downstream of adequate promoters and are preferably linked with transcription termination signals on the 3′ side. Nucleic acids thus prepared can be introduced into desired animals (cells) using known methods. The sequences of the antisense nucleic acids are preferably complementary to a gene or portion thereof encoding a CSPG core protein, synthetase, desulfation enzyme-suppressing protein, or sulfotransferase that is endogenous to the animals (cells) to be transformed with them. However, the sequences need not be perfectly complementary, as long as the antisense nucleic acids can effectively suppress expression of a gene. The transcribed RNAs preferably have 90% or higher, and most preferably 95% or higher complementarity to target gene transcripts. To effectively inhibit target gene expression using antisense nucleic acids, the antisense nucleic acids are preferably at least 15 nucleotides long, and less than 25 nucleotides long. However. the lengths of the antisense nucleic acids of the present invention are not limited to the lengths mentioned above, and they may be 100 nucleotides or more, or 500 nucleotides or more.

The antisense nucleic acids of the preset invention are not particularly limited, and can be prepared, for example, based on the nucleotide sequence of a versican gene (GenBank Accession No: BC096495; SEQ ID NO: 3), C4ST-1 (GenBank Accession No: NM_{—}021439; SEQ ID NO: 45), C4ST-2 (GenBank Accession NO: NM_{—}021528; SEQ ID NO: 47), C4ST-3 (GenBank Accession NO: XM_{—}355798; SEQ ID NO: 49), or such.

Expression of the above-mentioned genes encoding CSPG core proteins, synthetases, desulfation enzyme-suppressing proteins, or sulfotransferases can also be inhibited using ribozymes or ribozyme-encoding DNAs. Ribozymes refer to RNA molecules with catalytic activity. There are various ribozymes with different activities. Among others, studies that focused on ribozymes functioning as RNA-cleaving enzymes have enabled the design of ribozymes that cleave RNAs in a site-specific manner. Some ribozymes have 400 or more nucleotides, such as group I intron type ribozymes and Ml RNA, which is comprised by RNase P, but others, called hammerhead and hairpin ribozymes, have a catalytic domain of about 40 nucleotides (Koizumi, M. and Otsuka E., Tanpakushitsu Kakusan Koso (Protein, Nucleic Acid. and Enzyme), 1990, 35, 2191).

For example, the autocatalytic domain of a hammerhead ribozyme cleaves the sequence G13U14C15 at the 3′ side of C15. Base pairing between U14 and A9 has been shown to be essential for this activity, and the sequence can be cleaved when C15 is substituted with A15 or U15 (Koizumi, M. et al., FEBS Lett., 1988, 228, 228). Restriction enzyme-like RNA-cleaving ribozymes that recognize the sequence UC, U, or UA in target RNAs can be created by designing their substrate-binding sites to be complementary to an RNA sequence adjacent to a target site (Koizumi, M. et al., FEBS Lett., 1988, 239, 285; Koizumi, M, and Otsuka, E., Tanpakushitsu Kakusan Koso (Protein, Nucleic Acid, and Enzyme), 1990, 35, 2191; and Koizumi, M. et al., Nucl Acids Res., 1989, 17, 7059).

In addition, hairpin ribozymes are also useful for the purposes of the present invention. Such ribozymes are found in, for example, the minus strand of satellite RNAs of tobacco ring spot viruses (Buzayan, J. M., Nature, 1986, 323, 349). It has been shown that target-specific RNA-cleaving ribozymes can also be created from hairpin ribozymes (Kikuchi, Y. and Sasaki, N., Nucl Acids Res., 1991, 19, 6751; and Kikuchi, Y Kagaku to Seibutsu (Chemistry and Biology), 1992, 30, 112). Thus, the expression of the above-described genes encoding CSPG core proteins, synthetases, desulfation enzyme-suppressing proteins, or sulfotransferases can be inhibited by using ribozymes to specifically cleave the gene transcripts.

The expression of endogenous genes can also be suppressed by RNA interference (hereinafter abbreviated as “RNAi”), using double-stranded RNAs comprising a sequence the same as or similar to a target gene sequence.

A great many disease-related genes have been rapidly identified since the entire human nucleotide sequence was revealed upon the recent completion of the genome project, and currently specific gene-targeted therapies and drugs are being actively developed. Of these, the application to gene therapy of small interfering RNAs (siRNAs), which produce the effect of specific post-transcriptional suppression, has been drawing attention. RNAi is a technology currently drawing attention in which double-stranded RNAs (dsRNAs) incorporated directly into cells suppress the expression of genes with sequences homologous to the dsRNAs. In mammalian cells, RNAi can be induced using short dsRNAs (siRNAs) and has many advantages: compared to knockout mice, RNAi has a stable effect, simple experiments, low costs, and so on.

Nucleic acids with inhibitory activity based on RNAi effect are generally referred to as siRNAs or shRNAs. RNAi is a phenomenon in which, when cells or such are introduced with short double-stranded RNAs (hereinafter abbreviated as “dsRNAs”) comprising sense RNAs that comprise sequences homologous to the mRNAs of a target gene, and antisense RNAs that comprise sequences homologous a sequence complementary thereto, the dsRNAs bind specifically and selectively to the target gene mRNAs, induce their disruption, and cleave the target gene, thereby effectively inhibiting (suppressing) target gene expression. For example, when dsRNAs are introduced into cells, the expression of genes with sequences homologous to the RNAs is suppressed (the genes are knocked down). As described above, RNAi can suppress the expression of target genes, and is thus drawing attention as a method applicable to gene therapy, or as a simple gene knockout method replacing conventional methods of gene disruption, which are based on complicated and inefficient homologous recombination. The RNAs to be used in RNAi are not necessarily perfectly identical to the genes or portions thereof that encode an above-described CSPG core protein, synthetase, desulfation enzyme-suppressing protein, or sulfotransferase; however, the RNAs are preferably perfectly homologous to the genes or portions thereof.

The targets of the siRNAs to be designed are not particularly limited, as long as they are genes encoding an above-described CSPG core protein, synthetase, desulfation enzyme-suppressing protein, or sulfotransferase. Any region of the gene can be a candidate for a target. For example, siRNAs may be prepared based on a nucleotide sequence of the versican gene (SEQ ID NO: 3), C4ST-1 gene (SEQ ID NO: 45), C4ST-2 gene (SEQ ID NO: 47), C4ST-3 gene (SEQ ID NO: 49), and such. More specifically, partial regions of such sequences may be used as candidates for the targets. For example, siRNAs may be prepared based on portions of the nucleotide sequences of a versican gene (SEQ ID NO: 57), C4ST-1 gene (SEQ ID NO: 58), C4ST-2 gene (SEQ ID NO: 59), C4ST-3 gene (SEQ ID NO: 60), C6ST-1 gene (SEQ ID NO: 61), C6ST-2 gene (SEQ ID NO: 62), GalNAc4ST-1 gene (SEQ ID NO: 63), GalNAc4ST-2 gene (SEQ ID NO: 64), GALNAC4S-6ST gene (SEQ ID NO: 65), or such. More specifically, examples of the siRNAs also include those targeted to the DNA sequences (SEQ ID NOs: 67 to 85) specifically shown herein.

The siRNAs can be introduced into cells by adopting methods of introducing cells with plasmid DNAs linked with siRNAs synthesized in vitro or methods that comprise annealing two RNA strands.

The two RNA molecules described above may be closed at one end or, for example, may be siRNAs with hairpin structures (shRNAs). shRNAs refer to short hairpin RNAs, which are RNA molecules with a stem-loop structure, since a portion of the single strand constitutes a strand complementary to another portion. Thus, molecules capable of forming an intramolecular RNA duplex structure are also included in the siRNAs of the present invention.

In a preferred embodiment of the present invention, the siRNAs of the present invention also include, for example, double-stranded RNAs with additions or deletions of one or a few RNAs in an siRNA which targets a specific DNA sequence (SEQ ID NOs: 67 to 85) shown herein and which can suppress the expression of versican, C4ST-1, C4ST-2, C4ST-3, or such via RNAi effect, as long as the double-stranded RNAs have the function of suppressing the expression of a gene encoding an above-described CSPG core protein, synthetase, desulfation enzyme-suppressing protein, or sulfotransferase.

The RNAs used in RNAi (siRNAs) do not need to be perfectly identical (homologous) to the genes encoding the above proteins or portions thereof, however, the RNAs are preferably perfectly identical (homologous).

Some details of the RNAi mechanism still remain unclear, but it is understood that an enzyme called “DICER” (a member of the RNase III nuclease family) is contacted with a double-stranded RNA and degrades it in to small fragments, called “small interfering RNAs” or “siRNAs”. The double-stranded RNAs of the present invention that have RNAi effect include such double-stranded RNAs prior to being degraded by DICER. Specifically, since even long RNAs that have no RNAi effect when intact can be degraded into siRNAs which have RNAi effect in cells, the length of the double-stranded RNAs of the present invention is not particularly limited.

For example, long double-stranded RNAs covering the full-length or near full-length mRNA of a gene encoding an above-described CSPG core protein, synthetase, desulfation enzyme-suppressing protein, or sulfotransferase can be pre-digested, for example, by DICER, and then the degradation products can be used as agents of the present invention. These degradation products are expected to contain double-stranded RNA (siRNA) molecules with RNAi effect. With this method, it is not necessary to specifically select the mRNA regions expected to have RNAi effect. In other words, it is not necessary to accurately determine regions with RNAi effect in the mRNAs of the genes described above.

The above-described “double-stranded RNAs capable of suppression via RNAi effect” can be suitably prepared by those skilled in the art based on nucleotide sequences of the above-described CSPG genes encoding core proteins, synthetases, desulfation enzyme-suppressing proteins, or sulfotransferases, which are targeted by the double-stranded RNAs. For example, the double-stranded RNAs of the present invention can be prepared based on the nucleotide sequence of SEQ ID NO: 67. In other words, it is within the range of ordinary trials for those skilled in the art to select an arbitrary consecutive RNA region in an mRNA that is a transcript of the nucleotide sequence of SEQ ID NO: 67, and prepare double-stranded RNA corresponding to the region. Those skilled in the art can also use known methods to properly select siRNA sequences with stronger RNAi effect from the mRNA sequence, which is the transcript of the nucleotide sequence of SEQ ID NO: 67. When one of the strands is already identified, those skilled in the art can readily determine the nucleotide sequence of the other strand (complementary strand). Those skilled in the art can appropriately prepare siRNAs using a commercially available nucleic acid synthesizer. Alternatively, general custom synthesis services may be used to synthesize desired RNAs.

The siRNAs of the present invention are not necessarily single pairs of double-stranded RNAs directed to target sequences, but may be mixtures of multiple double-stranded RNAs directed to regions that cover the target sequence. Herein, those skilled in the art can appropriately prepare the siRNAs as nucleic acid mixtures matched to a target sequence by using a commercially available nucleic acid synthesizer or DICER enzyme. Meanwhile, general custom synthesis services may be used to synthesize desired RNAs. The siRNAs of the present invention include so-called “siRNA cocktails”.

All nucleotides in the siRNAs of the present invention do not necessarily need to be ribonucleotides (RNAs). Specifically, one or more of the ribonucleotides constituting the siRNAs of the present invention may be replaced with corresponding deoxyribonucleotides. The term “corresponding” means that although the sugar moieties are structurally differently, the nucleotide residues (adenine, guanine, cytosine, or thymine (uracil)) are the same. For example, deoxyribonucleotides corresponding to ribonucleotides with adenine refer to deoxyribonucleotides with adenine. The term “or more” described above is not particularly limited, but preferably refers to a small number of about two to five ribonucleotides.

Furthermore, DNAs (vectors) capable of expressing the RNAs of the present invention are also included in the preferred embodiments of compounds capable of suppressing the expression of the genes encoding the above-described proteins of the present invention. The DNAs (vectors) capable of expressing the double-stranded RNAs of the present invention are, for example, DNAs structured such that a DNA encoding one strand of a double-stranded RNA and a DNA encoding the other strand of the double-stranded RNA are linked with promoters so that each DNA can be expressed. The above DNAs of the present invention can be appropriately prepared by those skilled in the art using standard genetic engineering techniques. More specifically, the expression vectors of the present invention can be prepared by adequately inserting DNAs encoding the RNAs of the present invention into various known expression vectors.

Furthermore, the expression-inhibiting substances of the present invention also include compounds that inhibit the expression of the above-described CSPG core proteins, synthetases, desulfation enzyme-suppressing proteins, or sulfotransferases by binding to an expression regulatory region of a gene encoding the above-described CSPG core proteins, synthetases, desulfation enzyme-suppressing proteins, or sulfotransferases (for example, a promoter region; specific examples include the nucleotide sequence of SEQ ID NO: 66, which is a promoter region of PG-Lb). Such compounds can be obtained, for example, using a fragment of a promoter DNA of the gene encoding an above-described CSPG core protein, synthetase, desulfation enzyme-suppressing protein, or sulfotransferase to perform screening methods using as an indicator the activity of binding to the DNA fragment. Those skilled in the art can appropriately determine whether compounds of interest inhibit the expression of the above-described genes encoding CSPG core proteins, synthetases, desulfation enzyme-suppressing proteins, or sulfotransferases by using known methods, for example, reporter assays and such.

Furthermore, DNAs (vectors) capable of expressing the above-described RNAs of the present invention are also included in preferred embodiments of the compounds capable of inhibiting the expression of a gene encoding an above-described CSPG core protein, synthetase, desulfation enzyme-suppressing protein, or sulfotransferase of the present invention. For example, DNAs (vectors) capable of expressing the above-described double-stranded RNAs of the present invention are structured such that a DNA encoding one strand of a double-stranded RNA and a DNA encoding the other strand of the double-stranded RNA are linked to promoters so that both can be expressed. Those skilled in the art can appropriately prepare the above-described DNAs of the present invention using standard genetic engineering techniques. More specifically, the expression vectors of the present invention can be prepared by appropriately inserting DNAs encoding the RNAs of the present invention into various known expression vectors.

Preferred embodiments of the above-described vector of the present invention include vectors expressing RNAs (siRNAs) that can suppress the expression of versican, C4ST-1, C4ST-2, C4ST-3, or the like by RNAi effect.

Antibodies that bind to the above-described CSPG core proteins, synthetases, desulfation enzyme-suppressing compounds, or sulfotransferases can be prepared by methods known to those skilled in the art. Polyclonal antibodies can be obtained, for example, by the following procedure: small animals such as rabbits are immunized with an above-described natural protein or a recombinant protein expressed in microorganisms as a fusion protein with GST, or a partial peptide thereof. Sera are obtained from these animals and purified by, for example, ammonium sulfate precipitation, Protein A or G column, DEAE ion exchange chromatography, affinity column coupled with the core protein, synthetase, desulfation enzyme-suppressing compound, or sulfotransferase for CSPGs described above, synthetic peptide, or such, to prepare antibodies. Monoclonal antibodies can be obtained by the following procedure: small animals such as mice are immunized with an above-described CSPG core protein, synthetase, desulfation enzyme-suppressing compound, or sulfotransferase, or a partial peptide thereof. Spleens are removed from the mice and crushed to isolate cells. The cells are fused with mouse myeloma cells using a reagent such as polyethylene glycol. Clones producing antibodies that bind to an above-described CSPG core protein, synthetase, desulfation enzyme-suppressing compound, or sulfotransferase are selected from among the resulting fused cells (hybridomas). The obtained hybridomas are then transplanted in the peritoneal cavities of mice, and ascites is collected from the mice. The obtained monoclonal antibodies can be purified by, for example, ammonium sulfate precipitation, Protein A or G columns, DEAE ion exchange chromatography, affinity columns coupled with an above-described CSPG core protein, synthetase, desulfation enzyme-suppressing compound, or sulfotransferase, synthetic peptides, or such.

The antibodies of the present invention are not particularly limited as long as they bind to an above-described core protein, synthetase, desulfation enzyme-suppressing compound, or sulfotransferase of the present invention. The antibodies of the present invention may be human antibodies, humanized antibodies created by gene recombination, fragments or modified products of such antibodies, in addition to the polyclonal and monoclonal antibodies described above.

The proteins of the present invention used as sensitizing antigens to prepare antibodies are not limited in terms of the animal species from which the proteins are derived. However, the proteins are preferably derived from mammals, for example, mice and humans. Human-derived proteins are particularly preferred. The human-derived proteins can be appropriately obtained by those skilled in the art using the gene or amino acid sequences disclosed herein.

In the present invention, the proteins to be used as sensitizing antigens may be whole proteins or partial peptides thereof. Such partial peptides of the proteins include, for example. amino-terminal (N) fragments and carboxyl-terminal (C) fragments of the proteins. Herein, “antibodies” refer to antibodies that react with a full-length protein or fragment thereof.

In addition to immunizing nonhuman animals with antigens to obtain the above hybridomas, human lymphocytes, for example, EB virus-infected human lymphocytes, can be sensitized in vitro with the proteins or with cells expressing the proteins, or with lysates thereof, and the sensitized lymphocytes can be fused with human-derived myeloma cells with the ability to divide permanently, for example, U266, to obtain hybridomas that produce desired human antibodies with binding activity to the proteins.

It is expected that antibodies against the above-described CSPG core proteins, synthetases, desulfation enzyme-suppressing compounds, or sulfotransferases of the present invention exhibit the effect of inhibiting protein expression or function by binding to the proteins. When using the prepared antibodies for human administration (antibody therapy), the antibodies are preferably human or humanized antibodies in order to reduce immunogenicity.

Furthermore, in the present invention, low-molecular-weight substances (low-molecular-weight compounds) that bind to the above-described CSPG core proteins, synthetases, desulfation enzyme-suppressing compounds, or sulfotransferases are also included in the substances capable of inhibiting the function of the above-described CSPG core proteins, synthetases, desulfation enzyme-suppressing compounds, or sulfotransferases. Such low-molecular-weight substances may be natural or artificial compounds. In general, the compounds can be produced or obtained by methods known to those skilled in the art. The compounds of the present invention can also be obtained by the screening methods described below.

In addition, the substances of the present invention capable of inhibiting the expression or function of the above-described CSPG core proteins, synthetases, desulfation enzyme-suppressing proteins, or sulfotransferases include dominant-negative mutants (dominant-negative proteins) for the above-described CSPG core proteins, synthetases, desulfation enzyme-suppressing proteins, or sulfotransferases. The “dominant-negative protein mutants for the above CSPG core proteins, synthetases, desulfation enzyme-suppressing proteins, or sulfotransferases” refer to proteins with the function of reducing or abolishing the activity of endogenous wild-type proteins by expressing the genes encoding the CSPG core proteins, synthetases, desulfation enzyme-suppressing proteins, or sulfotransferases. Such dominant-negative proteins include, for example, versican core protein mutants that competitively inhibit the linking of the wild-type versican core protein with chondroitin sulfate.

Furthermore, in the present invention, the organs, tissues, or cells where the production or accumulation of chondroitin sulfate proteoglycans is inhibited are not specifically limited; however, they are preferably respiratory system organs, tissues or cells involved in obstructive ventilatory impairment, and are more preferably the lung or bronchi.

Compounds that inhibit the production or accumulation of chondroitin sulfate proteoglycans are expected to serve as therapeutic or preventive agents for chronic obstructive pulmonary diseases. Herein, “therapeutic or preventive” does not necessarily refer to a perfect therapeutic or preventive effect on organs, tissues, or cells with chronic obstructive pulmonary disease, and may refer to a partial effect.

In the present invention, the chronic obstructive pulmonary diseases are not specifically limited, as long as they are associated with obstructive ventilatory impairment; however, they are preferably pulmonary emphysema, chronic bronchitis and the like.

The obstructive ventilatory impairment-suppressing agents of the present invention have the activity of suppressing obstructive ventilatory impairment through inhibiting the production or accumulation of chondroitin sulfate proteoglycans, which is a cause of obstructive ventilatory impairment. Thus, preferred embodiments of the present invention provide, for example, therapeutic agents for pulmonary emphysema or chronic bronchitis which comprise as an active ingredient an obstructive ventilatory impairment-suppressing agent of the present invention.

The “obstructive ventilatory impairment-suppressing agents” of the present invention can also be referred to as “therapeutic agents for obstructive ventilatory impairment”, “obstructive ventilatory impairment-improving agents”. or the like. Meanwhile, the “suppressing agents” of the present invention can also be referred to as “pharmaceutical agents”, “pharmaceutical compositions”, “therapeutic medicines”, or the like.

The “treatments” of the present invention also comprise preventive effects that can suppress the onset of obstructive ventilatory impairment in advance. The treatments are not limited to those producing a perfect therapeutic effect on cells (tissues) developing obstructive ventilatory impairment, and the effects may be partial.

The agents of the present invention can be combined with physiologically acceptable carriers, excipients, diluents and such, and orally or parenterally administered as pharmaceutical compositions. Oral agents may be in the form of granules, powders, tablets, capsules, solutions, emulsions, suspensions, or the like. The dosage forms of parenteral agents can be selected from injections, infusions, external preparations, inhalants (nebulizers), suppositories, and the like. Injections include preparations for subcutaneous, intramuscular, intraperitoneal, intracranial, and intranasal injections, and the like. The external preparations include nasal preparations, ointments, and such. Techniques for formulating the above-described dosage forms that contain the agents of the present invention as primary ingredients are known.

For example, tablets for oral administration can be produced by compressing and shaping the agents of the present invention in combination with excipients, disintegrants, binders, lubricants, and the Like. Excipients commonly used include lactose, starch, mannitol, and the like. Commonly used disintegrants include calcium carbonate, carboxymethylcellulose calcium, and the like. Binders include gum arabic, carboxymethylcellulose, and polyvinylpyrrolidone. Known lubricants include talc, magnesium stearate, and such.

Known coatings can be applied to tablets comprising the agents of the present invention to prepare enteric coated formulations or for masking. Ethylcellulose, polyoxyethylene glycol, or such can be used as a coating agent.

Meanwhile, injections can be prepared by dissolving the agents of the present invention, which are chief ingredients, together with an appropriate dispersing agent, or dissolving or dispersing the agents in a dispersion medium. Both water-based and oil-based injections can be prepared, depending on the selection of dispersion medium. When preparing water-based injections, the dispersing agent is distilled water, physiological saline, Ringer's solution or such. For oil-based injections, any of the various vegetable oils, propylene glycols, or such is used as a dispersing agent. If required, a preservative such as paraben may be added at this time. Known isotonizing agents such as sodium chloride and glucose can also be added to the injections. In addition, soothing agents such as benzalkonium chloride and procaine hydrochloride can be added.

Alternatively, the agents of the present invention can be formed into solid, liquid, or semi-solid compositions to prepare external preparations. Such solid or liquid compositions can be prepared as the same compositions as described above and then used as external preparations. The semi-solid compositions can be prepared using an appropriate solvent, to which a thickener is added if required. Water, ethyl alcohol, polyethylene glycol, and the like can be used as the solvent. Commonly used thickeners are bentonite, polyvinyl alcohol, acrylic acid, methacrylic acid, polyvinylpyrrolidone, and the like. Preservatives such as benzalkonium chloride can be added to these compositions. Alternatively, suppositories can be prepared by combining the compositions with carriers, like oil bases such as cacao butter, or aqueous gel bases such as cellulose derivatives.

When the agents of the present invention are used as gene therapy agents, the agents may be directly administered by injection, or vectors carrying the nucleic acid may be administered. Such vectors include adenovirus vectors, adeno-associated virus vectors, herpes virus vectors, vaccinia virus vectors, retroviral vectors, and lentivirus vectors. These vectors allow efficient administration.

Alternatively, the agents of the present invention can be encapsulated into phospholipid vesicles such as liposomes, and then the vesicles can be administered. Vesicles carrying siRNAs or shRNAs are introduced into given cells by lipofection. The resulting cells are then systemically administered, for example, intravenously or intra-arterially. The cells can also be locally administered into tissues or such with obstructive ventilatory impairment. siRNAs exhibit a quite superior and specific post-transcriptional suppression effect in vitro; however, in vivo they are rapidly degraded due to serum nuclease activity, and thus, their time was limited. There is therefore demand for the development of optimized and effective delivery systems. As one example, Ochiya et al. have reported that atelocollagen, a bio-affinity material, is a highly suitable siRNA carrier because it has the activity of protecting nucleic acids from nucleases in the body when mixed with the nucleic acids to form a complex (Ochiya, T. et al., Nat. Med., 1999, 5, 707-710; Ochiya, T. et al., Curr. Gene Ther., 2001, 1, 31-52); however, the methods for introducing drugs of the present invention are not limited thereto.

The agents of the present invention are administered to mammals including humans at required (effective) doses, within a dose range considered to be safe. Ultimately, the doses of the agents of the present invention can be appropriately determined by medical practitioners or veterinarians after considering the dosage form and administration method, and the patient's age and weight, symptoms, and the like. For example, adenoviruses are administered once a day at a dose of about 106 to 1013 viruses every one to eight weeks, although the doses vary depending on the age, sex, symptoms, administration route, administration frequency, and dosage form.

Commercially available gene transfer kits (for example: AdenoExpressTM, Clontech) may be used to introduce siRNAs or shRNAs into target tissues or organs.

When the agents of the present invention are used, the type of disease and site to which the agents are applied are not particularly limited, as long as the disease develops obstructive ventilatory impairment; for example, the agents are applied to pulmonary emphysema, chronic bronchitis and such. The above diseases may occur in combination with other diseases.

The present invention also provides methods of screening for obstructive ventilatory impairment-suppressing agents, wherein the methods comprise selecting, from test samples, substances with the activity of inhibiting the production or accumulation of chondroitin sulfate proteoglycans. Obstructive ventilatory impairment-suppressing agents or candidate compounds for obstructive ventilatory impairment-suppressing agents can be efficiently obtained using the screening methods of the present invention.

Preferred embodiments of the screening methods of the present invention are methods of screening for obstructive ventilatory impairment-suppressing agents that comprise the step of selecting substances with any of the activities (a) to (d):

(a) promoting the degradation of chondroitin sulfate proteoglycans;
(b) inhibiting the synthesis of chondroitin sulfate proteoglycans;
(c) desulfating chondroitin sulfate proteoglycans; and
(d) inhibiting the sulfation of chondroitin sulfate proteoglycans.

Representative examples based on fundamental principles common in screening for these substances include methods comprising the following procedure:

A preferred procedure uses tools (1) to (3) below, and is as follows: (1) and (2) are mixed in a test tube or culture dish, and the resulting effect is simply detected using (3).
(1) chondroitin sulfate proteoglycans (CSPGs) themselves, or glycosaminoglycans (GAG) chains, or cells synthesizing (producing) CSPGs or GAG chains
(2) test compounds (for example, enormous compound libraries owned by pharmaceutical companies)
(3) methods for detecting CSPG cleavage sites, the amount of CSPGs, or the amount of free glycosaminoglycans (GAGs)

Embodiments of the screening methods of the present invention are exemplified below. In the embodiments described below, the chondroitin sulfate proteoglycans, synthetases, compounds suppressing desulfation enzymes, sulfotransferases, degradation-promoting enzymes, and desulfation enzymes to be used include those derived from humans, mice, rats, and others, but are not limited thereto. Chondroitin sulfate proteoglycan portions are components such as glycosaminoglycan chains or core proteins, or portions thereof. The chondroitin sulfate proteoglycan portions are not particularly limited.

The test compounds to be used in the embodiments described below are not particularly limited, but include, for example, single compounds, such as natural compounds, organic compounds, inorganic compounds, proteins, and peptides, as well as compound libraries, expression products of gene libraries, cell extracts, cell culture supernatants, products of fermenting microorganisms, extracts of marine organisms, and plant extracts.

In the embodiments described below. the “contact” with test compounds is typically achieved by mixing the test compounds with chondroitin sulfate proteoglycans, portions thereof, synthetases, compounds suppressing desulfation enzymes, sulfotransferases, degradation-promoting enzymes, or desulfation enzymes, but the “contact” is not limited to this methods. For example, the “contact” can also be achieved by contacting test compounds with cells expressing these proteins or portions thereof.

In the embodiments described below, the “cells” include those derived from humans, mice, rats, and such, but are not limited thereto. Cells of microorganisms, such as Escherichia coli and yeasts, which are transformed to express the proteins used in each embodiment, can also be used. For example, the “cells that express chondroitin sulfate proteoglycans” include cells that express endogenous genes for chondroitin sulfate proteoglycans, and cells that express introduced foreign genes for chondroitin sulfate proteoglycans. Such cells that express foreign genes for chondroitin sulfate proteoglycans can typically be prepared by introducing host cells with expression vectors carrying a chondroitin sulfate proteoglycan gene as an insert. The expression vectors can be prepared using standard genetic engineering techniques.

The “chondroitin sulfate proteoglycan core proteins” described below include, for example, core proteins of matrix-type chondroitin sulfate proteoglycans, such as aggrecan, versican, neurocan, and brevican, and core proteins of membrane chondroitin sulfate proteoglycans, such as decorin, biglycan, fibromodulin, and PG-Lb. The “synthetases” include, for example, GalNAc4ST-1, GalNAc4ST-2, GALNAC4S-6ST, UA2OST, GalT-I, GalT-II, GlcAT-I, and XylosylT. The “sulfotransferases” include, for example, chondroitin D-N-acetylgalactosamine-4-0-sulfotransferase 1 (C4ST-1), chondroitin D-N-acetylgalactosamine-4-O-sulfotransferase 2 (C4ST-2), chondroitin D-N-acetylgalactosamine-4-O-sulfotransferase 3 (C4ST-3), D4ST, C6ST-1, and C6ST-2. The “degradation-promoting enzymes” include, for example, ADAMTS-1, ADAMTS-4, ADAMTS-5, chondroitinase ABC (ChABC), chondroitinase AC, chondroitinase B, and calpain I. The “desulfation enzymes” include, for example, chondroitin-4-sulfatase and chondroitin-6-sulfatase.

Embodiments of the screening methods of the present invention include methods comprising the step of selecting compounds that have the activity of promoting the degradation of chondroitin sulfate proteoglycans. An example of the above-mentioned methods of the present invention comprises the steps of:

(a) contacting test compounds with chondroitin sulfate proteoglycans or portions thereof;
(b) measuring the abundance of chondroitin sulfate proteoglycans or portions thereof; and
(c) selecting substances that reduce the abundances as compared with those determined in the absence of the test compounds.

In the above methods, first, test compounds are contacted with chondroitin sulfate proteoglycans or portions thereof.

In these methods, the amount of the chondroitin sulfate proteoglycans or portions thereof is then measured. The measurement can be conducted by methods known to those skilled in the art. For example, the amounts can be detected using labeled compounds or antibodies that bind to the chondroitin sulfate proteoglycans or portions thereof, and then measuring the amount of the label. Alternatively, the detection can be achieved by chromatography or mass spectrometry.

In these methods, compounds that reduce the abundance of the chondroitin sulfate proteoglycans or portions thereof as compared with in the absence of a test compound (the control) are then selected. Compounds resulting in a reduction can be used as therapeutic agents for obstructive ventilatory impairment.

Below is a brief illustrative example of the methods able to assess (measure) whether a test compound has the above activity (a): the activity of promoting the degradation of chondroitin sulfate proteoglycans.

Embodiment of the methods for screening for the above activity (a) of promoting the degradation of chondroitin sulfate proteoglycans:

A CS-GAG, such as chondroitin sulfate A (CS-A), CS-B, CS-C (Seikagaku Co., ICN, Sigma, and others), or human-derived proteoglycan (BGN, ISL, and others), is prepared, and 96-well plates are coated with it at a concentration of 10 μg/ml (using known methods, such as in Kawashima, H. et al., J. Biol. Chem, 2002, 277, 12921-12930). Various test compounds are added to each well of the plates. After two hours of reaction at 37° C., changes in CS-GAG are detected.

Detection methods include, for example, the simple WFA lectin (Wisteria floribunda lectin)-binding method. Since WFA lectin binds to the GalNAc residues of CS-GAG chains, it can easily detect CS-GAGs. Chondroitinase ABC is used as a positive control for test compounds. The principle behind this use of chondroitinase ABC is that its addition degrades CS-GAG chains, making it impossible for WFA lectin to bind them. More specifically, FITC-labeled WFA lectin (EY Co.) is added to the CS-coated wells before and after mixing the test compounds, and changes in the intensity of FITC fluorescence in the wells due to the CS-GAG degradation can be quantified and digitized very simply by using detection devices, such as fluorescence plate readers or fluorescence microscopes. Compounds whose addition most reduces fluorescent values may be determined to be novel therapeutic candidate compounds that fulfill the concept of the present invention.

In an alternative detection method, anti-CS antibody (clone CS56, Seikagaku Co.) can be used to directly label CS-GAGs. As with WFA lectin, large-scale screening can be carried out simply and in very short time by adding FITC-labeled anti-CS antibody to CS-coated wells and examining changes in fluorescence value.

In more specific detection methods, GAG content is accurately quantified and digitized by simply using the plates before and after mixing of test compounds in an sGAG Assay Kit (Wieslab Co.), an ELISA Kit for Sulphanated Glycosaminoglycans (Funakoshi Co.), or such.

More specifically, the reducing ends of free GAG chains can easily be fluorescently labeled by adding 2-aminobenzamide, 2-aminopyridine (2-AB and 2-AP, respectively; LUD Co. and others), or the like to the plates before and after mixing of test compounds, which enables more specific analysis using HPLC, MALDI-MS, LC-MS, or such to determine the types of sugar chains and even the content of each type of chain. These methods, which examine the properties of candidate compounds in detail, take screening to the next level.

Other embodiments of the screening methods of the present invention include methods comprising the step of selecting substances with the activity of inhibiting the synthesis of chondroitin sulfate proteoglycans. These methods of the present invention comprise, for example, the steps of:

(a) contacting test compounds with cells expressing chondroitin sulfate proteoglycans or portions thereof, extracts of these cells, or groups of substances including those enzymes and substrates constituting the process of chondroitin sulfate proteoglycan synthesis;
(b) measuring the amount of synthesized chondroitin sulfate proteoglycans or intermediates thereof in the above-mentioned cells, cell extracts, or group of substances; and
(c) selecting compounds that reduce the amount as compared to in the absence of the test compounds.

In the above methods, test compounds are first contacted with cells expressing chondroitin sulfate proteoglycans or portions thereof, extract of these cells, or groups of substances including those enzymes and substrates that constitute the process of chondroitin sulfate proteoglyean synthesis.

Next, the amount of synthesized chondroitin sulfate proteoglycans or intermediates thereof is measured. The measurement can be performed by those skilled in the art using known methods; for example, methods using labeled antibodies, mass spectrometry, and chromatography can be used.

Further, compounds that reduce (suppress) the synthesized amount as compared with in the absence of the test compounds (the control) are selected. Compounds resulting in a reduction (suppression) can be used as therapeutic agents for obstructive ventilatory impairment.

Below is a brief illustrative example of the methods able to assess (measure) whether a test compound has the above activity (b): the activity of inhibiting the synthesis of chondroitin sulfate proteoglycans.

Embodiment of the methods for screening for the above activity (b) of inhibiting the synthesis of chondroitin sulfate proteoglycans:

Cells and cell lines synthesizing chondroitin sulfate are known to researchers in the art. In human, for example, chondroitin sulfate is produced after 16 hours of cell culture by standard methods for culturing mononuclear cells isolated from peripheral blood collected from healthy subjects (Uhlin-Hansen, L. et al., Blood, 1993, 82, 2880 etc.). Alternatively, for more convenience, there are many examples of known cell; for example, the fibroblast cell line NIH3T3 (Phillip, H. A., et al., J. Biol. Chem., 2004, 279, 48640 etc.); the renal tubule-derived cancer cell line ACHN (Kawashima, H. et al., J. Biol. Chem., 2002, 277, 12921), the renal distal tubule-derived cell line MDCK (Borges, F. T. et al., Kidney Int., 2005, 68, 1630 etc.), and the vascular endothelial cell line HUVEC (Schick, B. P. et al., Blood, 2001, 97, 449 etc.). Various test compounds are added during the process of culturing such cell lines for set periods, and changes in the amount of CS-GAG before and after culture can be easily evaluated by the above-described method of (a). Compounds that suppress the increase in the amount of CS-GAG after culture (which thus reflects the amount of synthesized CS-GAG) can be easily determined to be candidate therapeutic compounds that fulfill the concept of the present invention.

As a further option, cell lines constitutively expressing the genes for CS-GAG synthetases such as GalNAc4ST-1 and XylosylT can be prepared by introducing the genes into CHO cells, L cells, or such by well-known methods. The use of such cell lines that constitutively synthesize CS-GAG allows more clear determination of candidates for therapeutic compounds.

In another embodiment, the screening methods of the present invention include methods comprising the step of selecting substances with the activity of desulfating chondroitin sulfate proteoglycans. The above methods of the present invention comprise, for example, the steps of:

(a) contacting test compounds with chondroitin sulfate proteoglycans or portions thereof;
(b) measuring the amount of sulfation in the chondroitin sulfate proteoglycans or portions thereof; and
(c) selecting substances that reduce the amount of sulfation as compared with in the absence of the test compounds.

In the above methods, test compounds are first contacted with chondroitin sulfate proteoglycans or portions thereof.

Next, the amount of sulfation in the chondroitin sulfate proteoglycans or portions thereof is measured. The measurement can be conducted using methods known to those skilled in the art. For example, the amount of sulfation can be determined by using labeled compounds or antibodies that bind to the desulfated structures remaining in the chondroitin sulfate proteoglycans or portions thereof, and measuring the amount of the label. Alternatively, the measurement can be achieved by chromatography or mass spectrometry or such.

Then, in the present methods compounds that reduce the abundances of the chondroitin sulfate proteoglycans or portions thereof as compared with in the absence of the test compounds (the control) are selected. Compounds resulting in a reduction can be used as therapeutic agents for obstructive ventilatory impairment.

Below is a brief illustrative example of the methods able to assess (measure) whether a test compound has the above activity (c): the activity of desulfating chondroitin sulfate proteoglycans.

Embodiment of the methods for screening for the above activity (c) of desulfating chondroitin sulfate proteoglycans:

By using essentially the same method as described above in (a), human-derived proteoglycans (BGN Co., ISL Co., etc.) or such are prepared and coated on to 6-well plates at a concentration of 10 μg/ml (by known methods, such as those described in Kawashima H. et al., J. Biol. Chem. 2002, 277, 12921-12930). Various test compounds are added to each well of the plates, and alterations in CS-GAG are detected after two hours of reaction at 37° C.

In this detection method, desulfated moieties can be easily detected using the reaction of either anti-proteoglycan Δdi4S antibody (clone: 2-B-6, which recognizes sulfated moieties at position 4) or anti-proteoglycan Δdi6S antibody (clone: 3-B-3, which recognizes sulfated moieties at position 6) (both from Seikagaku Co.) with the disaccharide structure of the desulfated fragments that remain in the core protein of proteoglycans after desulfation. Thus, FITC-labeled 2-B-6 or 3-B-3 antibody is reacted in such plates before and after mixed culture, and changes in the fluorescence value can be simply detected. Compounds whose addition increases the fluorescence intensity can be determined to be substances that promote desulfation, and are easily identified as novel candidate therapeutic compounds that fulfill the concept of the present invention.

Another embodiment of the screening methods of present invention includes methods comprising the step of selecting substances with the activity of inhibiting the sulfation of chondroitin sulfate proteoglycans. The above methods of the present invention comprise, for example, the steps of:

(a) contacting test compounds with cells expressing chondroitin sulfate proteoglycans or portions thereof, extracts of these cells, or groups of substances including those enzymes and substrates constituting the process of sulfation of chondroitin sulfate proteoglycans;
(b) measuring the activity of sulfation of chondroitin sulfate proteoglycans in the above-mentioned cells, cell extracts, or groups of substances; and
(c) selecting compounds that reduce the activity as compared with in the absence of the test compounds.

In the above methods, test compounds are first contacted with chondroitin sulfate proteoglycans or portions thereof.

Next, the amount of sulfation in the chondroitin sulfate proteoglycans or portions thereof is measured. The measurement can be conducted using methods known to those skilled in the art. For example, the amount of sulfation can be determined by using labeled compounds or antibodies that bind to the sulfated structures of the chondroitin sulfate proteoglycans or portions thereof, and measuring the amount of the label. Alternatively, the measurement can be achieved by chromatography or mass spectrometry and such.

Then, compounds that reduce the abundance of the chondroitin sulfate proteoglycans or portions thereof as compared with in the absence of the test compounds (the control) are selected. Compounds resulting in a reduction can be used as therapeutic agents for obstructive ventilatory impairment.

Below is a brief illustrative example of the methods able to assess (measure) whether a test compound has the above activity (d): the activity of inhibiting the sulfation of chondroitin sulfate proteoglycans.

Embodiment of the methods for screening for the above activity (d) of inhibiting the sulfation of chondroitin sulfate proteoglycans:

The cells and cell lines that promote the sulfation of chondroitin sulfates are the same as described above in (c). Various test compounds are mixed during a set period of culture of such cell lines, and the degree of sulfation before and after the culture can be easily determined by, for example, using an antibody that recognizes sulfation at position 4 (clone: LY111) or an antibody that recognizes sulfation at position 6 (clone: MC21C) (both from Seikagaku Co.). Fluorescence values may be compared between before and after the culture by using fluorescently labeled antibodies. Alternatively, the same detection method as described above in (c) can be conducted using 2-B-6 or 3-B-3 antibodies before and after culture. Compounds that suppress an increase in the sulfation after cell culture (an increase in the fluorescence value for LY111 or MC21C), or compounds that promote the progression of desulfation after cell culture (an increase in the fluorescence value for 2-B-6 or 3-B-3) can be easily determined to be candidate therapeutic compounds that fulfill the concept of the present invention.

As a further option, cell lines that constitutively express sulfotransferase genes such as C4ST-1 and C6ST-1 can be prepared by introducing the genes into CHO cells, L cells, or such by well-known methods. The use of such cell lines that constitutively add sulfate groups allows more clear determination of candidates for therapeutic compounds.

Other preferred embodiments of the present invention are methods of screening for obstructive ventilatory impairment-suppressing agents in which compounds that reduce the expression level of a gene encoding a CSPG core protein, synthetase, desulfation enzyme-suppressing protein, or sulfotransferase of the present invention, or compounds that increase the expression level of a gene for an enzyme that desulfates CSPGs or promotes the degradation of CSPGs, are selected; wherein the method comprises the steps of:

(a) contacting test compounds with cells expressing a gene encoding a CSPG core protein, synthetase, desulfation enzyme-suppressing protein, sulfotransferase, degradation-promoting enzyme, or desulfating enzyme;
(b) determining the expression level of the gene in the cells;
(c) comparing the expression level with that in the absence of the test compounds (the control); and
(d) selecting compounds that reduce the expression level of the gene of the CSPG core protein, synthetase, desulfation enzyme-suppressing protein, or sulfotransferase as compared with the control, or compounds that increase the expression level of the gene of the CSPG desulfating enzyme or the CSPG degradation-promoting enzyme as compared with the control.

In the above methods, test compounds are first contacted with cells expressing a gene encoding a CSPG core protein, synthetase, desulfating enzyme-suppressing protein, sulfotransferase, degradation-promoting enzyme, or desulfating enzyme.

Next, the expression level of the gene encoding the core protein, synthetase, desulfating enzyme-suppressing protein, sulfotransferase, degradation-promoting enzyme, or desulfating enzyme is measured. Herein, “expression of the gene” includes both transcription and translation. Gene expression level can be measured by methods known to those skilled in the art.

For example, mRNAs are extracted from cells expressing any one of the above-described proteins by conventional methods, and these mRNAs can be used as templates in Northern hybridization, RT-PCR, DNA arrays, or such to measure the transcription level of the gene. Alternatively, protein fractions are collected from cells expressing a gene encoding any of the above-described proteins, and expression of the protein can be detected by electrophoresis such as SDS-PAGE to measure the level of gene translation. Alternatively, the level of gene translation can be measured by detecting the expression of any of the above-described proteins by Western blotting using an antibody against the proteins. Such antibodies for use in detecting the proteins are not particularly limited, as long as they are detectable. For example, both monoclonal and polyclonal antibodies can be used.

Next, the expression level is compared with that in the absence of the test compounds (the control).

Then, when the gene encodes a CSPG core protein, synthetase, desulfating enzyme-suppressing protein, or sulfotransferase, compounds that reduce (suppress) the expression level of the gene as compared with a control are selected. The compounds resulting in a reduction (suppression) can be agents for suppressing obstructive ventilatory impairment or candidate compounds for treating obstructive ventilatory impairment.

Alternatively, when the gene encodes a CSPG desulfating enzyme or an enzyme promoting CSPG degradation, compounds that increase (enhance) the expression level of the gene as compared with a control are selected. Compounds resulting in an increase (enhancement) can be agents for suppressing obstructive ventilatory impairment or candidate compounds for treating obstructive ventilatory impairment.

An embodiment of the screening methods the present invention is a method in which compounds that reduce the expression level of a gene encoding a CSPG core protein, synthetase, desulfation enzyme-suppressing protein, or sulfotransferase of the present invention, or compounds that increase the expression level of a gene for a CSPG degradation-promoting enzyme or a CSPG desulfating enzyme, can be selected using the expression of a reporter gene as an indicator. The above methods of the present invention comprise, for example, the steps of:

(a) contacting test compounds with cells or cell extracts containing a DNA structured such that a reporter gene is operably linked to a transcriptional regulatory region of a gene encoding a CSPG core protein, synthetase, desulfating enzyme-suppressing protein, sulfotransferase, degradation-promoting enzyme, or desulfating enzyme;
(b) measuring the expression level of the reporter gene;
(c) comparing the level with the that in the absence of the test compounds (the control); and
(d) selecting compounds that reduce the expression level of the reporter gene as compared with the control when the reporter gene is operably linked with a transcriptional regulatory region of the gene encoding a CSPG core protein, synthetase, desulfating enzyme-suppressing protein, or sulfotransferase; or selecting compounds that increase the expression level of the reporter gene as compared with the control when the reporter gene is operably linked with a transcriptional regulatory region of a gene encoding a CSPG degradation-promoting enzyme or a CSPG desulfating enzyme.

In the above methods, test compounds are first contacted with cells or cell extracts containing DNAs structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme.

Herein, “operably linked” means that a reporter gene is linked with a transcriptional regulatory region of a gene encoding a CSPG core protein, synthetase, desulfating enzyme-suppressing protein, sulfotransferase, degradation-promoting enzyme, or desulfating enzyme, such that expression of the reporter gene is induced upon binding of transcriptional factors to the transcriptional regulatory region. Therefore, the meaning of “operably linked” also includes cases where a reporter gene is linked with a different gene and produces a fusion protein with a different gene product, as long as expression of the fusion protein is induced upon the binding of transcriptional factors to the transcriptional regulatory region of the gene encoding the CSPG core protein, synthetase, desulfating enzyme-suppressing protein, sulfotransferase, degradation-promoting enzyme, or desulfating enzyme. Those skilled in the art can obtain the transcriptional regulatory regions of genes encoding CSPG core proteins, synthetases, desulfating enzyme-suppressing proteins, sulfotransferases, degradation-promoting enzymes, or desulfating enzymes that are present in the genome, based on the cDNA nucleotide sequences of the genes encoding the CSPG core proteins, synthetases, desulfating enzyme-suppressing proteins, sulfotransferases, degradation-promoting enzymes, or desulfating enzymes.

The reporter genes for use in these methods are not particularly limited, as long as their expression is detectable. The reporter genes include, for example, the CAT gene, the lacZ gene, the luciferase gene, and the GFP gene. The “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include, for example, cells introduced with vectors carrying such structures as inserts. Such vectors can be prepared by methods well known to those skilled in the art. The vectors can be introduced into cells by standard methods, for example, calcium phosphate precipitation, electroporation, lipofection, and microinjection. The “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include cells in which the structure has been integrated into the chromosomes. A DNA structure can be integrated into chromosomes by methods generally used by those skilled in the art, for example, gene transfer methods using homologous recombination.

The “cell extracts containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme” include, for example, mixtures of cell extracts included in commercially available in vitro transcription-translation kits and DNAs structured such that a reporter gene is operably linked with the transcriptional regulatory region of the gene encoding a CSPG core protein, synthetase, desulfating enzyme-suppressing protein, or sulfotransferase.

“Contact” can be achieved by adding test compounds to a culture medium of “cells containing a DNA structured such that a reporter gene is operably linked with a transcriptional regulatory region of a gene encoding a CSPG core protein, synthetase, desulfating enzyme-suppressing protein, sulfotransferase, degradation-promoting enzyme, or desulfating enzyme”, or by adding test compounds to the above-described commercially available cell extracts containing the DNAs. When the test compound is a protein, contact may also be achieved, for example, by introducing a DNA vector expressing the protein into the cells.

In the above methods, the expression level of the reporter gene is then measured. The expression level of the reporter gene can be measured by methods known to those skilled in the art, depending on the type of the reporter gene. When the reporter gene is the CAT gene, its expression can be determined, for example, by detecting the acetylation of chloramphenicol by the gene product. When the reporter gene is the lacZ gene, its expression level can be determined by detecting the color development of chromogenic compounds due to the catalytic action of the gene expression product. Alternatively, when the reporter gene is the luciferase gene, its expression level can be determined by detecting the fluorescence of fluorogenic compounds due to the catalytic action of the gene expression product. Furthermore, when the reporter gene is the GFP gene, its expression level can be determined by detecting the fluorescence of the GFP protein.

In the above methods, the expression level of the reporter gene is then compared with that in the absence of the test compounds (the control).

In the present methods, compounds that reduce (suppress) the expression level of a reporter gene as compared with a control are then selected, where the reporter gene is operably linked with a gene encoding a CSPG core protein, synthetase, desulfating enzyme-suppressing protein, or sulfotransferase. Compounds resulting in a reduction (suppression) can be agents for suppressing obstructive ventilatory impairment or candidate compounds for treating obstructive ventilatory impairment.

Alternatively, when the reporter gene is operably linked with a gene encoding a CSPG degradation-promoting enzyme or CSPG desulfating enzyme, compounds that increase (enhance) the reporter gene expression level as compared with a control are selected. Compounds resulting in an increase (enhancement) can be agents for suppressing obstructive ventilatory impairment or candidate compounds for treating obstructive ventilatory impairment.

The obstructive ventilatory impairment-suppressing agents that are found by the screening methods of the present invention are preferably therapeutic or preventive agents for chronic obstructive pulmonary diseases.

The present invention also provides kits comprising various agents, reagents, and the like, which are used to conduct the screening methods of the present invention.

The kits of the present invention can be prepared, for example, by selecting adequate reagents from the above-described various reagents, depending on the screening method to be conducted. The kits of the present invention may contain, for example, the chondroitin sulfate proteoglycans of the present invention. The kits of the present invention may further contain various reagents, vessels, and the like to be used in the methods of the present invention. The kits may appropriately contain, for example, anti-chondroitin sulfate proteoglycan antibodies, probes, various reaction reagents, cells, culture media, control samples, buffers, and instruction manuals containing a description of how to use the kits.

Preferred embodiments of the present invention are the methods of screening for obstructive ventilatory impairment-suppressing agents, comprising the step of detecting whether the production or accumulation of chondroitin sulfate proteoglycans is inhibited. Thus, the kits for screening for the obstructive ventilatory impairment-suppressing agents of the present invention may contain, for example, oligonucleotides such as probes for the genes encoding CSPG core proteins, and primers to amplify certain regions of these genes; and antibodies recognizing CSPGs (anti-chondroitin sulfate proteoglycan antibodies), which can be used to detect chondroitin sulfate proteoglycans.

The above-described oligonucleotides specifically hybridize to, for example, DNAs of the genes encoding the versican core protein of the present invention. Herein, “specifically hybridize to” means that the oligonucleotides do not significantly cross-hybridize to DNAs encoding other proteins under standard hybridization conditions, and preferably under stringent hybridization conditions (for example, the conditions described in Sambrook J. et al. “Molecular Cloning” 2nd Ed., Cold Spring Harbour Laboratory Press, New York, USA, 1989). The oligonucleotides are not necessarily perfectly complementary to the nucleotide sequences of the versican core protein genes of the present invention, as long as they allow specific hybridization.

The hybridization conditions in the present invention include, for example, conditions such as “2× SSC, 0.1% SDS, and 50C”, “2× SSC, 0.1% SDS, and 42° C”, and “1× SSC, 0.1% SDS, and 37° C.”, and more stringent conditions such as “2× SSC, 0.1% SDS, and 65° C”, “0.5× SSC, 0.1% SDS, and 42° C”, and “0.2× SSC, 0.1% SDS, and 65° C”. More specifically, for methods using Rapid-hyb buffer (Amersham Life Science), prehybridization is carried out at 68° C. for 30 minutes or more; then a probe is added, and after one hour or more of hybrid formation at 68° C., washing is carried out three times with 2× SSC/0.1% SDS at room temperature for 20 minutes, then three times with 1× SSC/0.1% SDS at 37° C. for 20 minutes, and finally twice with 1× SSC/0.1% SDS at 50° C. for 20 minutes. Alternatively, for example, prehybridization is carried out in Expresshyb Hybridization Solution (CLONTECH) at 55° C. for 30 minutes, and then a labeled probe is added thereto, and after one hour or more of incubation at 37-55° C., washing is carried out three times in 2× SSC/0.1% SDS at room temperature for 20 minutes and then once in lx SSC/0.1% SDS at 37° C. for 20 minutes. More stringent conditions can be achieved, for example, by increasing the temperature for prehybridization, hybridization, or second washing. For example, temperatures for prehybridization and hybridization can be 60° C., and more stringent conditions can be achieved by increasing the temperature to 68° C. In addition to conditions such as the salt concentration of the buffers and the temperature, those skilled in the art can also determine conditions using other conditions including the nucleotide sequence composition of the probe, the probe length and concentration, and reaction time.

The oligonucleotides can be used as probes and primers in the above-described screening kits of the present invention. When the oligonucleotides are used as primers, they are typically 15 to 100 bp long, and preferably 17 to 30 bp long. Such primers are not particularly limited as long as they can amplify at least a portion of a DNA of the above-described genes of the present invention.

The present invention also provides therapeutic or preventive methods for chronic obstructive pulmonary diseases, which comprise the step of administering the agents of the present invention to individuals (for example, to patients and such).

The individuals subjected to the therapeutic or preventive methods of the present invention are not particularly limited, as long as they are organisms that can develop a chronic obstructive pulmonary disease; however, humans are preferred.

In general, administration to individuals can be achieved, for example, by methods known to those skilled in the art, such as intraarterial injections, intravenous injections, and subcutaneous injections. The administered dose varies depending on the patient's weight and age, and the administration method or such; however, those skilled in the art (medical practitioners, veterinarians, pharmacists, and the like) can appropriately select a suitable dose.

The present invention also relates to the uses of agents of the present invention in producing obstructive ventilatory impairment -suppressing agents.

All prior-art documents cited herein are incorporated by reference herein.

### EXAMPLES

There is no previous report on actual clinical treatment that involves observing the in vivo dynamics of proteoglycans including CSPG and changes in the pathological condition according to such regulation. As one such example, the present invention focuses on chondroitinase ABC, which is a CSPG degradation enzyme.

RNAi is a phenomenon discovered by Fire et al in 1998 (Fire, A., Nature, 1998, 391, 806-811), where double-strand RNA strongly suppresses expression of homologous target genes. RNAi has been drawing attention recently as an applicable method in gene therapy, because it is simpler than conventional gene transfer methods using vectors or such, and its target specificity is high. However, RNA is rapidly degraded in the body due to serum nuclease activity, and thus maintaining the effect for a long time was previously thought to be difficult. Recent reports (Takeshita, F., PNAS., 2003, 102(34), 12177-82; Minakuchi, Y., Nucleic Acids Research, 2004, 32(13), e109) describe that bovine skin-derived atelocollagen is highly suitable as a carrier of siRNA, because it forms a complex with nucleic acids and has an effect of protecting nucleic acids from degradation enzymes in the body. As with the case of chondroitinase ABC described above, no previous report has described controlling the in vivo dynamics of CSPG by suppressing the expression of C6ST or GalNAc using RNAi.

Chondroitinase ABC is a lyase classified under the enzyme number EC.4.2.2.4 (chondroitin ABC lyase). Chondroitinase ABC has an activity of degrading the 4-deoxy-β-D-gluc-4-enuronosyl group of the polysaccharides having a 1,4-β-D-hexosaminide linkage, and 1,3-β-D-glucuronosyl linkage or 1,3-α-L-iduronosyl linkage. There is no report on using chondroitinase ABC to treat pulmonary emphysema or COPD.

The present invention also focuses on an approach different from that which uses chondroitinase ABC (the CSPG degradation enzyme described above). This alternative approach focuses on chondroitin D-N-acetylgalactosamine-6-O-sulfotransferase 1 (C6ST-1), chondroitin D-N-acetylgalactosamine-6-O-sulfotransferase 2 (C6ST-2), chondroitin D-N-acetylgalactosamine-6-O-sulfotransferase 3 (C6ST-3), N-acetylgalactosamine-4-O-sulfotransferase 1 (GalNAc 4ST-1), N-acetylgalactosamine-4-O-sulfotransferase 2 (GalNAc 4ST-2), and N-acetylgalactosamine-4-O-sulfotransferase (GALNAC 4S-6ST), all of which are enzymes involved in the biosynthesis of CSPG. In the Examples, the present invention tried to use a mechanism different from that of chondroitinase ABC to regulate CSPG in vivo, and performed RNA interference (RNAi) by introducing genes (small interfering RNA (siRNA)) to specifically inhibit genes that express these two types of enzymes.

Herein below, the present invention will be specifically described with reference to Examples, but the technical scope of the present invention is not to be construed as being limited thereto.

### Example 1

### The Effect of Chondroitinase ABC in Suppressing Emphysematous Lesions in Pulmonary Emphysema Model Mice

A standard mouse model of pulmonary emphysema made by intratracheal administration of porcine pancreatic elastase (PPE) was used in this Example. This classical model is widely used as a pulmonary emphysema model because of its superior reproducibility and simplicity.

(Non-patent Documents: Karlinsky, J. B. et al., Am Rev Respir Dis., 1978, 117, 1109-1133; Otto-Verbeme, C. J. et al., Protective effect of pulmonary surfactant on elastase-induced emphysema in mice, Eur Respir. J., 1992, 5, 1223-1230; Janoff, A. et al., Prevention of elastase-induced experimental emphysema by oral administration of a synthetic elastase inhibitor, Am Rev Respir Dis., 1980, 121, 1025-1029; Christensen, T. G. et al., Irreversible bronchial goblet cell metaplasia in hamsters with elastase-induced panacinar emphysema, J Clin Invest., 1977, 59, 397-404; Lucey, E. C. et al., Remodeling of alveolar walls after elastase treatment of hamsters: results of elastin and collagen mRNA in situ hybridization, Am J Respir Crit Care Med., 1998, 158, 555-564; Snider, G. L. Lucey, E. C. Stone, P. J., Animal models of emphysema, Am Rev Respir Dis., 1986, 133, 149-169.)

In this Example, the effect of chondroitinase ABC in suppressing emphysematous lesions was examined by hematoxylin-eosin staining (HE staining) of lung tissue samples from pulmonary emphysema model mice.

First, the model mice were prepared. PPE (4 units; Calbiochem-Novabiochem) was administered intratracheally to C57BL6/J mice (female, 5- to 6-weeks old; CLEA Japan). The mice were grown for three weeks after administration, and then lung tissues were collected from them. Mice that did not have PPE administration were used as the control group.

Prior to PPE administration, chondroitinase ABC (20 units/ml; Sigma Aldrich) was administered at a dose of 4 units/head to the peritoneal cavities of the mice. Additional chondroitinase ABC was administered after PPE treatment.

The collected right lung tissues were embedded in the OCT compound (Miles), an embedding medium for cryosectioning, and cryoblocks were prepared using liquid nitrogen. The cryoblocks were sliced into 6-μm sections using cryostat (Microm).

The resulting sections were fixed with 1% glutaraldehyde (Nacalai Tesque) for 10 minutes, and further fixed with formol-calcium solution for 10 minutes. The sections were washed with phosphate buffer, and then stained with Lillie-Mayer's hematoxylin solution (Sigma Aldrich Japan) at room temperature for 5 minutes. The sections were washed gently with a decolorizing solution (70% ethanol containing 0.5% HCl; prepared using reagents from Nacalai Tesque), and then washed with water for 10 minutes. The sections were stained with eosin-alcohol at room temperature for 5 minutes, and then washed with water for 10 minutes. The sections were washed gently with 100% ethanol, and then allowed to stand for 3 minutes. The sections were further washed gently with xylene (Nacalai Tesque), and allowed to stand for 10 minutes. This sample was histologically observed using a light microscope (Leica Microsystems).

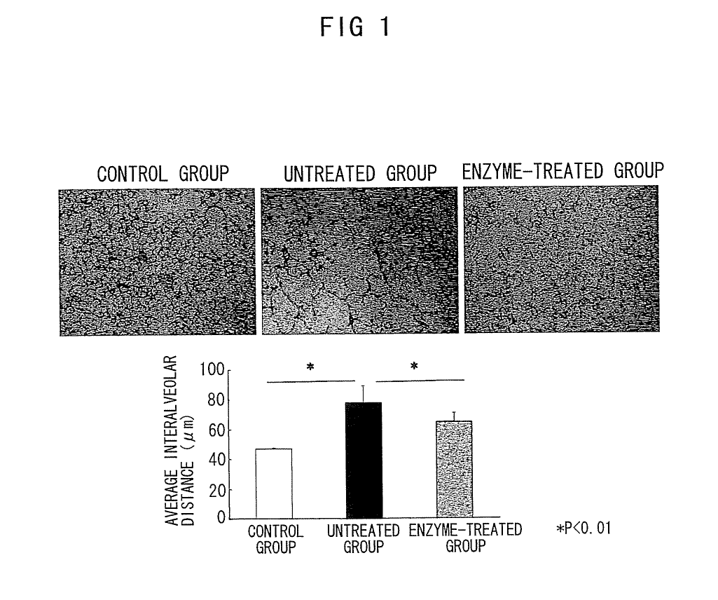

The obtained histological images are shown in the upper panel of FIG. 1. The histological features of a normal lung parenchyma with characteristic faveolate alveolar septal walls are found in the control group (PPE non-administered mice). Meanwhile, emphysematous lesions due to destruction and abolishment of alveolar septal walls and enlargement of air space (characteristics of pulmonary emphysema) can be observed in the untreated group (administered with PPE but not with chondroitinase ABC) shown in the middle photograph. On the other hand, in the enzyme-treated group (administered with PPE and chondroitinase ABC), slight abolishment of alveolar septal walls and emphysematous lesions can be observed; however, their levels are significantly improved.

Meanwhile, the enlargement of alveolar space is known to be one of the characteristic features of pulmonary emphysema. As shown in the upper panel of FIG. 1, the enlargement can be found occasionally in the HE-stained image of a pulmonary emphysema model mouse. Determining the average interalveolar distance is a known method for numerically evaluating the degree of pathological change in a pulmonary emphysema model mouse (Thurlbeck, W. M., Thorax., 1967, 22, 483-96; Murakami, S., Am J Respir Crit Care Med., 2005, 172, 581-9). Photographs of the HE-stained samples were taken at a magnification of ×400 using the same light microscope used above (Leica Microsystems). The therapeutic effect of chondroitinase ABC was evaluated by comparing the average interalveolar distances in the photographs of the control group, untreated group and treated group.

The results of the average interalveolar distances measured are shown in the lower panel of FIG. 1. The average interalveolar distance in the control group was 47.125+0.65 μm. Meanwhile, the distance was found to be significantly increased in the untreated group: 77.85±11.42 μm (vs. the control group, P=0.002; t test). On the other hand, the average interalveolar distance in the enzyme-treated group was 64.925±0.31 μm (vs. the untreated group, P=0.0013; t test). Thus, the average interalveolar distance was increased as compared with that in the control group; however, it was significantly decreased as compared with that in the untreated group.

As described above, the results of this Example revealed that the in vivo administration of chondroitinase ABC improved emphysematous lesions or suppressed their progression in the intratracheal PPE administration-induced pulmonary emphysema model mice.

### Example 2

### The Effect of Chondroitinase ABC in Suppressing the Deposition of Proteoglycans in Pulmonary Emphysema Model Mice

In this Example, the proteoglycan-suppressing effect of chondroitinase ABC was examined and compared using lung tissue samples from pulmonary emphysema model mice. The sections obtained by the same method as in Example 1 were fixed in acetone (Sigma Aldrich Japan) for ten minutes, and then washed with phosphate buffer. An anti-chondroitin sulfate proteoglycans (CSPG) antibody (clone CS56, mouse monoclonal antibody, 10 μg/ml; Seikagaku) was added as the primary antibody, and the sections were reacted at room temperature for one hour. Then, the secondary antibody reaction was conducted using a Histofine Mouse Stain Kit (Nichirei; used for mouse monoclonal antibodies), and DAB substrate (Nichirei) was added thereto for the enzymatic color reaction. The samples were observed under a light microscope (Leica Microsystems).

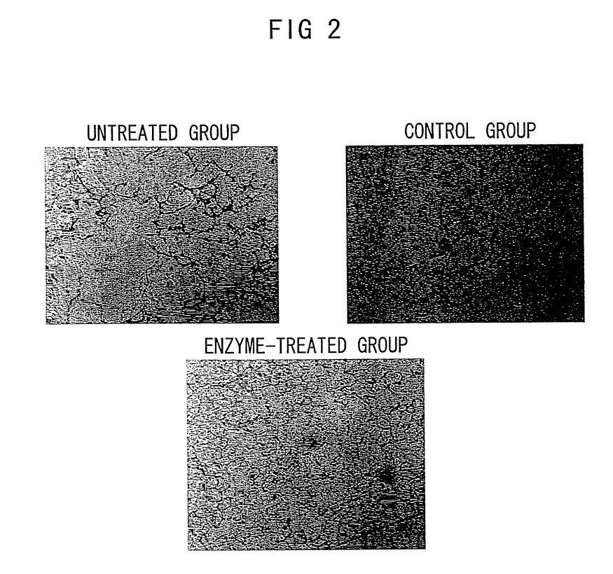

The obtained histological images are shown in FIG. 2. In the control group, some positive brown signals are seen in the alveolar septal walls, as well as in the surrounding areas of bronchiole in the middle of the photograph; however, a particularly intense deposition of CSPG can not be observed. Meanwhile, in the untreated group, strong positive signals, namely intense deposition of CSPG, are observed in the regions of destroyed alveolar septal walls as seen in Example 1. On the other hand, in the enzyme-treated group, the degree of the destruction of alveolar septal walls is smaller, and positive signals are nearly as strong as those in the control group.

The above results, which were obtained by immunostaining lung tissues with an anti-CSPG antibody, revealed that the in vivo administration of chondroitinase ABC suppresses the deposition of CSPG through degradation in the lung tissues of the intratracheal PPE administration-induced pulmonary emphysema model mice.

### Example 3

### The Effect of Chondroitinase ABC in Suppressing the Accumulation of Proteoglycans in Pulmonary Emphysema Model Mice

The CSPG deposited as shown in Example 2 is known to adsorb chemokines, which are substances that induce inflammatory cells such as macrophages in the body. It is assumed that the deposition of CSPG results in the attraction of inflammatory cells and thereby destroys lung tissues. Based on such assumption, this Example assessed the effect of chondroitinase ABC on the accumulation dynamics of alveolar macrophages by immunohistochemical staining of lung tissue samples from pulmonary emphysema model mice.

Sections obtained by the procedures described in Example 1 were fixed with acetone (Sigma Aldrich Japan) for 10 minutes, and washed with phosphate buffer. A rat-derived anti-mouse macrophage antibody (clone F4/80, at 1:200 dilution; BMA) was added as a primary antibody, and the sections were incubated at room temperature for 1 hour. Then, a peroxidase-labeled donkey-derived anti-rat IgG (at 1:200 dilution; Biosource) was added as a secondary antibody, and the sections were incubated at room temperature for 30 minutes. After the incubation, DAB substrate (Nichirei) was added to the samples. The samples were observed using a light microscope (Leica Microsystems).

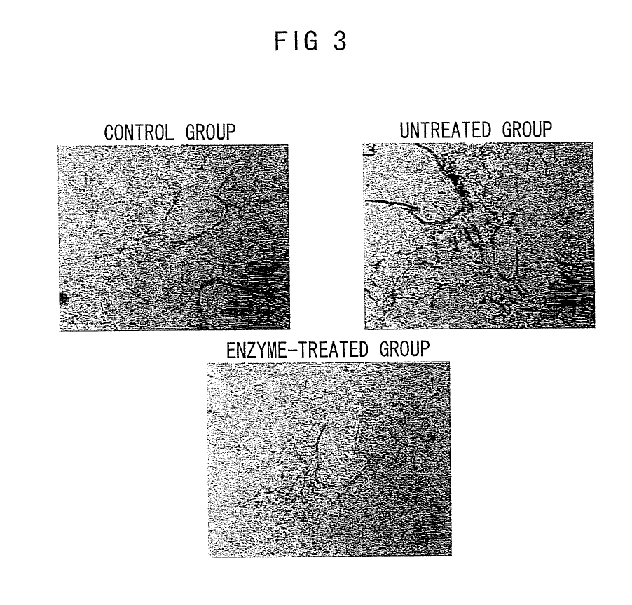

The obtained histological images are shown in FIG. 3. In the control group, some positive signals are observed in the alveolar region. Meanwhile, in the untreated group, aggregation of positive signals, namely the accumulation of alveolar macrophages, is observed extensively in the areas surrounding the bronchiole in the middle of the photograph. On the other hand, in the enzyme-treated group, positive signals are nearly as strong as those in the control group.

The above results of the anti-macrophage antibody immunostaining of lung tissues revealed that the induced accumulation of macrophages in lung tissues of the intratracheal PPE administration-induced pulmonary emphysema model mice was significantly suppressed by in vivo administration of chondroitinase ABC. The results described in this Example and in Example 2 suggest that chondroitinase ABC suppresses the CSPG deposition induced by intratracheal PPE administration, thereby suppressing the accumulation of alveolar macrophages. It is concluded that suppressing the accumulation of macrophages results in the suppression of emphysematous lesions.

### Example 4

### The Effects of Chondroitinase ABC, the C6ST Antisense Agent, and the GalNAc Antisense Agent in Suppressing the Deposition of Sulfated Glycosaminoglycans in Pulmonary Emphysema Model Mice

This Example evaluates the effect of suppressing the deposition of sulfated glycosaminoglycans (sGAG) in the left lung tissues of pulmonary emphysema model mice. Like other proteoglycans, CSPG, which is a target molecule of the present invention, is constituted by covalent bonding with sGAG. Thus, the purpose of this Example is to numerically evaluate the effects of chondroitinase ABC, a C6ST antisense agent, and a GalNAc antisense agent in suppressing the deposition of CSPG in lung tissues by quantifying sGAG. This was carried out by using alcian blue binding reaction-based sGAG assay. The pulmonary emphysema model mice were prepared by the same method as used in Examples 1 to 3. After administration, the mice were grown for three weeks and lung tissues were then collected.

The C6ST antisense agent and GalNAc antisense agent were administered by the following procedure: 1 μg of siRNA GalNAc or siRNA C6ST (GeneWorld) was combined with 1% atelocollagen (Koken Co.), which is an siRNA vehicle, and administered to the peritoneal cavities once a week after PPE administration. The dose was 200 μl/head.

*[GalNAc4ST-1 siRNA cocktail sequences]
(GenBank accession number NM_75140)

| | |
|---|---|
| 5′-accccacaactcggaacgatgcggct-3′ | (SEQ ID NO: 67) |
| 5′-tgcatgttctcgtccatcctgctg-3′ | (SEQ ID NO: 68) |
| 5′-cgccaccgtgtactgtactgtgaagt-3′ | (SEQ ID NO: 69) |
| 5′-aggctgctccaactggaagagggtg-3′ | (SEQ ID NO: 70) |

*[GalNAc4ST-2 siRNA cocktail sequences]
(GenBank accession number NM_199055)

| | |
|---|---|
| 5′-atatagtatctaggatatatgtag-3′ | (SEQ ID NO: 71) |
| 5′-gaagtaccaaaagctggctgctcta-3′ | (SEQ ID NO: 72) |
| 5′-ttctatcacttggactatttgatgtt-3′ | (SEQ ID NO: 73) |
| 5′-tacacaactccacatttgtaatttg-3′ | (SEQ ID NO: 74) |

*[GALNAC4S-6ST siRNA cocktail sequences]
(GenBank accession number NM_029935)

| | |
|---|---|
| 5′-ccagaagccaagctcattgttatg-3′ | (SEQ ID NO: 75) |
| 5′-ctgtggagaggttgtactcagacta-3′ | (SEQ ID NO: 76) |
| 5′-atttgcctggaagacaacgtgagagc-3′ | (SEQ ID NO: 77) |
| 5′-gtcccttctgcagaagctgggcccact-3′ | (SEQ ID NO: 78) |

*[C6ST-1 siRNA cocktail sequences]
(GenBank accession number NM_016803)

| | |
|---|---|
| 5′-gcgccccctctccccatggagaaag-3′ | (SEQ ID NO: 79) |
| 5′-gctttgcctcaggatttccgggacc-3′ | (SEQ ID NO: 80) |
| 5′-ggttcagccttggtctaccgtgatgtc-3′ | (SEQ ID NO: 81) |
| 5′-gcagttgttgctatgcgacctgtat-3′ | (SEQ ID NO: 82) |

*[C6ST-2 siRNA cocktail sequences]
(GenBank accession number NM_021715)

| | |
|---|---|
| 5′-tggggagagtgaggattcggtgaa-3′ | (SEQ ID NO: 83) |
| 5′-cggacgtgggactcgtcgaggacaaag-3′ | (SEQ ID NO: 84) |
| 5′-cgaaagtacctgcccgcccgtttcgc-3′ | (SEQ ID NO: 85) |

A therapeutic method using chondroitinase ABC was carried out by administering, prior to PPE treatment, chondroitinase ABC (20 units/ml; Sigma Aldrich) to the peritoneal cavities of the mice at a dose of 4 units/head. Additional chondroitinase ABC was administered after PPE treatment.

In the method of sample processing, left lungs were collected at the same time when the lung tissues used in Examples 1 and 2 were collected. The left lungs were washed with phosphate buffer, and soaked in 2 ml of 0.2% Triton X (Sigma Aldrich Japan) and then allowed to stand on ice. The lungs were homogenized with a homogenizer, and the homogenates were stirred at 4° C. overnight. The sGAG contents in the left lungs were determined using the tissue suspensions as samples and an sGAG quantitation kit (Wieslab Co.). The alcian blue binding reaction-based sGAG assay was carried out according to the document attached to the sGAG quantitation kit.

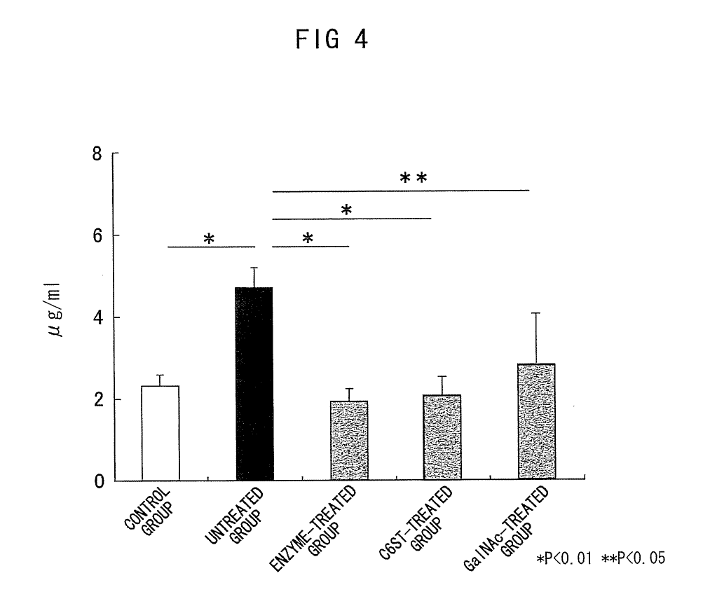

The results of this Example are shown in FIG. 4. The amount of sGAG deposited in the left lung was 2.31±0.28 μg/Lt lung for the control group (PPE non-administered). Meanwhile, the amount in the untreated group (PPE administered, therapeutic agent non-administered) was found to be significantly increased: 4.71±0.49 μg/Lt lung (vs. the control group, P=0.00033; t test). These results correlate with the results obtained by the anti-CSPG staining of lung tissues shown in Example 2. On the other hand, the amounts of sGAG deposited in the enzyme-treated group (PPE-administered, chondroitinase ABC-administered), C6ST-treated group (PPE-administered, C6ST antisense agent-administered), and GalNAc-treated group (PPE-administered, GalNAc antisense agent-administered) were 1.93±0.31 μg/Lt lung (vs. the untreated group, P=0.0000036; t test), 2.06±0.28 μg/Lt lung (vs. the untreated group, P=0.0004; t test), and 2.81±1.24 μg/Lt lung (vs. the untreated group, P=0.018; t test), respectively. Thus, the amounts of sGAG deposited were significantly decreased as compared with that of the untreated group.

These results revealed that chondroitinase ABC, the C6ST antisense agent, and the GalNAc antisense agent suppress the intratracheal PPE administration-induced increase of sGAG deposit in the lungs.

As described above, chondroitinase ABC, the C6ST antisense agent, and the GalNAc antisense agent all either promote the degradation of CSPG or suppress its expression, which suggests that the mechanism of suppressing the intratracheal PPE administration-induced increase of sGAG deposit in the lungs as shown in this Example, was promotion of CSPG degradation or suppression of CSPG expression.

### Example 5

### The Effects of Chondroitinase ABC, the C6ST Antisense Agent, and the GalNAc Antisense Agent in Maintaining the Respiratory Function of Pulmonary Emphysema Model Mice

The above Examples describe the effects of chondroitinase ABC, a C6ST antisense agent, and a GalNAc antisense agent in suppressing emphysematous lesions in the pulmonary emphysema model mice by suppressing CSPG deposition. Meanwhile, in order to examine the clinical effects of chondroitinase ABC, the C6ST antisense agent, and the GalNAc antisense agent in the pulmonary emphysema model mice, this Example used static lung compliance (Cst) as an indicator to evaluate their effects on respiratory function. Cst represents lung tissue elasticity and it increases in pulmonary emphysema, which is a disease accompanied by tissue destruction in the alveolar region.

The pulmonary emphysema model mice were prepared by the same procedure as shown in Examples 1 to 4 above, and treated with chondroitinase ABC, the C6ST antisense agent, and the GalNAc antisense agent in the same way as described in Example 4. Spontaneous respiration of the mice was terminated by anesthesia, and Cst was measured using the pressure-volume (P-V) loop mode of the respiratory function analyzer Flexi Vent (SCIREQ). After terminating the spontaneous respiration, Flexi Vent was connected to a mouse by the following procedure: after median incision of the mouse, a special cannula was inserted into the trachea, and the areas surrounding the bronchi were ligated.

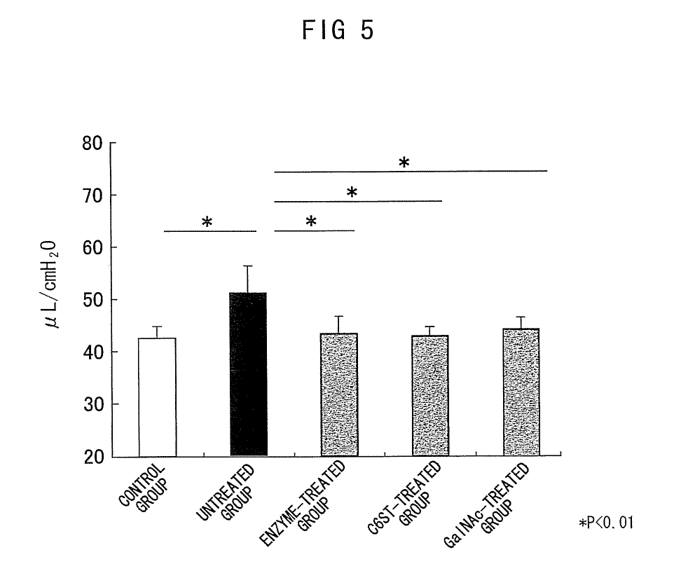

The results of this Example are shown in FIG. 5. Cst in the control group was 42.62±2.25 μl/cm H₂O. Meanwhile, Cst in the untreated group was 51.22±5.2 μl/cm H₂O (vs. the control group, P=0.03; t test); the increase was found to be statistically significant. In the enzyme-treated group, C6ST-treated group, and GalNAc-treated group, the Cst values were 43.41±3.39 μl/cm H₂O (vs. the untreated group, P=0.047; t test), 42.92±1.82 μl/cm H₂O (vs. the untreated group, P=0.03; t test), and 44.15±2.29 μl/cm H₂(vs. the untreated group, P=0.018; t test), respectively. All the Cst values were significantly decreased as compared with that in the untreated group.

These results revealed that chondroitinase ABC, the C6ST antisense agent, and the GalNAc antisense agent significantly suppressed the Cst increase in intratracheal PPE administration-induced pulmonary emphysema. The results of this Example also suggest that the administration of chondroitinase ABC, the C6ST antisense agent, or the GalNAc antisense agent not only suppressed the destruction of lung tissues but also had an actual effect of improving clinical symptoms (respiratory condition).

### Example 6

### The Effects of Chondroitinase ABC, the C6ST Antisense Agent, and the GalNAc Antisense Agent in Conserving Tissues of Pulmonary Emphysema Model Mice

The lung volume increases as an emphysematous lesion advances in pulmonary emphysema which has a characteristic pathological feature of enlarged alveolar air space. This Example was conducted to demonstrate that the therapeutic effects of chondroitinase ABC, the C6ST antisense agent, and the GalNAc antisense agent are not only the suppression of destruction at the cellular level, but they also extend to the morphological maintenance and conservation of organs.

The lung tissues used in this Example are the right lung of the lung tissues used in Example 5. The lung tissues extracted from mice were washed gently with phosphate buffer, and then placed in a glass vessel which was also filled with phosphate buffer. The weight of the glass vessel filled with phosphate buffer was measured in advance. The weight increase upon addition of the lung tissues into the vessel was converted into a liquid volume as the lung volume.

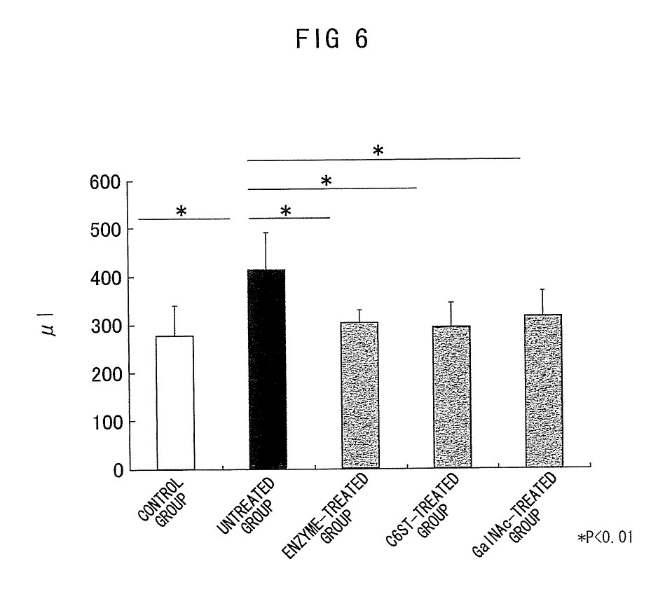

The results of this Example are shown in FIG. 6. The lung volume in the control group was 277.5±61.85 μl. Meanwhile, the volume in the untreated group was 413.33±77.67 μl (vs. the control group, P=0.024; t test); the increase was found to be statistically significant. On the other hand, the lung volumes in the enzyme-treated group, C6ST-treated group, and GalNAc-treated group were 303.33±25.17 μl (vs. the untreated group, P=0.04; t test), 292.5±51.23 μl (vs. the untreated group, P=0.027; t test), and 315±51.96 μl (vs. the untreated group, P=0.049; t test), respectively. The lung volumes were significantly decreased as compared with that of the untreated group.

These results revealed that chondroitinase ABC, the C6ST antisense agent, and the GalNAc antisense agent effectively suppress the lung volume increase in pulmonary emphysema induced by intratracheal PPE administration. The results described in this Example also indicate that the administration of chondroitinase ABC, the C6ST antisense agent, or the GalNAc antisense agent not only suppresses the destruction at the cellular level, but also has the effects of maintaining and conserving the morphology of organs and repairing destroyed tissues.

### INDUSTRIAL APPLICABILITY

Chondroitinase ABC, C6ST antisense agents, and GalNAc antisense agents of the present invention represent agents that suppress pulmonary emphysematous lesions and comprise as an active ingredient a substance having an activity of promoting CSPG degradation, inhibiting CSPG synthesis or inhibiting CSPG sulfation, and which are effective for treating and/or preventing COPD including pulmonary emphysema accompanied by destruction of alveolar walls and enlargement of alveolar space. The agents of the present invention for suppressing pulmonary emphysematous lesions are extremely useful in treating and/or preventing COPD, because they are efficacious in suppressing COPD-associated symptoms in various ways, such as by suppressing the destruction of alveolar walls and enlargement of alveolar space, suppressing the deposition of CSPG, suppressing the accumulation of alveolar macrophages, and maintaining the respiratory function in pulmonary emphysema model mice. The agents of the present invention for suppressing pulmonary emphysematous lesions, and the therapeutic and/or prevention methods using the agents are extremely effective means to improve the prognosis of COPD patients.

1. An obstructive ventilatory impairment-suppressing agent which comprises as an active ingredient a substance that inhibits the production or accumulation of a chondroitin sulfate proteoglycan.

2. The agent of claim 1, wherein the substance has an activity of promoting the degradation of a chondroitin sulfate proteoglycan.

3. The agent of claim 1, wherein the substance has an activity of inhibiting the synthesis of a chondroitin sulfate proteoglycan.

4. The agent of claim 1, wherein the substance has an activity of desulfating a chondroitin sulfate proteoglycan.

5. The agent of claim 1, wherein the substance has an activity of inhibiting the sulfation of a chondroitin sulfate proteoglycan.

6. The agent of claim 1, wherein the production or accumulation of a chondroitin sulfate proteoglycan is inhibited in a lung.

7. The agent of claim 1, which is used for treating or preventing a chronic obstructive pulmonary disease.

8. The agent of claim 7, wherein the chronic obstructive pulmonary disease is pulmonary emphysema.

9. The agent of claim 7, wherein the chronic obstructive pulmonary disease is chronic bronchitis.

10. A method of screening for an obstructive ventilatory impairment-suppressing agent, which comprises selecting from a test sample a substance with an activity of inhibiting the production or accumulation of a chondroitin sulfate proteoglycan.

11. The method of claim 10, which comprises the step of selecting a substance with the activity of any of:
(a) promoting the degradation of a chondroitin sulfate proteoglycan;
(b) inhibiting the synthesis of a chondroitin sulfate proteoglycan;
(c) desulfating a chondroitin sulfate proteoglycan; and
(d) inhibiting the sulfation of a chondroitin sulfate proteoglycan.

12. The method of claim 10, wherein the obstructive ventilatory impairment-suppressing agent is used for treating or preventing a chronic obstructive pulmonary disease.

13. A method of treating or preventing a chronic obstructive pulmonary disease, which comprises administering to a patient in need thereof a substance that inhibits the production or accumulation of a chondroitin sulfate proteoglycan.
